(19) United States
(12) Patent Application Publication
(10) Pub. No.: US 2014/0161827 A1
(43) Pub. Date: Jun. 12, 2014

(54) COMPOSITIONS AND METHODS FOR TREATING CANCER

(71) Applicants: Richard J. Santen, Ivy, VA (US); Sarah E. Aiyar, Tampa, FL (US)

(72) Inventors: Richard J. Santen, Ivy, VA (US); Sarah E. Aiyar, Tampa, FL (US)

(73) Assignee: University of Virginia Patent Foundation, Charlottesville, VA (US)

(21) Appl. No.: 14/116,433

(22) PCT Filed: May 9, 2012
(86) PCT No.: PCT/US2012/037056

§ 371 Date: Feb. 18, 2014

Related U.S. Application Data
(60) Provisional application No. 61/483,821, filed on May 9, 2011.

(51) Int. Cl. A61K 47/48; A61K 31/165; A61K 31/353; A61K 31/565; A61K 31/537; A61K 31/60; A61K 31/015

(52) U.S. Cl. CPC A61K 47/48669; A61K 31/60; A61K 31/165; A61K 31/015; A61K 31/565; A61K 31/537; A61K 31/353
USPC 424/178.1

(57) ABSTRACT

The present invention provides combination therapies useful for treating cancer, particularly breast cancer. The invention provides, in various embodiments, methods of treating a cancer, comprising administering to a patient afflicted therewith of an effective amount an immunoconjugate comprising a monoclonal antibody moiety and a first pro-apoptotic drug moiety linked thereto; and administering to the patient an effective amount of a second pro-apoptotic drug. The monoclonal antibody moiety of the immunoconjugate can act to target receptors of hormone-resistant breast cancer cells, such as HER2. Synergistic effects can be seen when the two pro-apoptotic drugs, acting by a common molecular mechanism (vertical modulation) or different molecular mechanisms (horizontal modulation) are administered to patients afflicted by breast cancer, such as hormone-resistant breast cancer.

### CROSS-REFERENCE TO RELATED APPLICATIONS

This application claims the priority of U.S. Ser. No. 61/483,821, filed May 9, 2011, the disclosure of which is incorporated by reference herein in its entirety.

### STATEMENT REGARDING FEDERALLY SPONSORED RESEARCH OR DEVELOPMENT

This invention was made with government support under Grant Nos. GG11284 awarded by The Department of Defense. The government has certain rights in the invention.

### BACKGROUND

The predicted mortality rate for breast cancer in the European Union for year 2011 is an estimated 75,688 deaths [1]. For the United states breast cancer deaths for 2010 was predicted to be 39,840 [2]. In approximately 70% of breast cancers the estrogen receptor (ER) is the key promoter of tumor proliferations, and therefore a first line treatment is to inhibit ER signaling through the use of tamoxifen or aromatase inhibitors, which block estrogen production [3]. However, initial (de novo) or subsequent (acquired) resistance of the cancer cells to the effect of these inhibitors, which is believed to occur by development of increased sensitivity to estrogen by the cancer cells through biological reprogramming, can limit the therapeutic benefits of estrogen lowering agents for many patients.

Two-thirds of women with estrogen receptor (ER) positive breast cancer respond to hormone therapy, which can be accomplished by removal of the ovaries, by administration of tamoxifen or aromatase (estrogen synthesis) inhibitors, and by use of compounds called GnRH super-agonist analogues. However, clinical observations have revealed that breast cancer cells can adapt to conditions of low estradiol by developing enhanced sensitivity to estradiol. Specifically, 200 pg/ml estradiol is required to stimulate tumor growth before acute deprivation of estradiol, whereas levels of 10-15 pg/ml are sufficient to cause tumor proliferation after adaptation 12-18 months later. Such hormone-resistant breast cancers are then unresponsive to continued aromatase therapy, and the cancer comprising these hormone-resistant cells can become uncontrollable.

Investigations of the growth of breast cells in culture have shown that when wild-type MCF-7 cells are cultured over a prolonged period in estrogen-free medium, the cells initially stop growing but then, months later, the cells adapt and grow as rapidly as wild-type MCF-7 cells maximally stimulated with estradiol. These adapted cultured cells, named LIED (long-term estrogen deprivation) cells, which are models for “hormone-resistant” or “hormone-refractory” cells as are responsible for loss of responsiveness of breast cancers to aromatase inhibitors, have been used to study processes relating to hormone adaptation. When mutations give rise to such cells in a patient, it is a significant negative development in their survival prospects.

Up-regulation of the estrogen receptor HER2 is observed after hormonal therapy. The trastuzumab-maytansinoid conjugate, T-DM1, is an antibody-drug conjugate comprising the HER2-specific humanized antibody trastuzumab covalently linked to the microtubule inhibitory agent DM1, an analog of maytansine. This conjugate has been shown to target HER2-positive breast cancer cells. See, for example, Lewis Phillips G D, Li G, Dugger D L, Crocker L M, Parsons K L, Mai E, Blattler W A, Lambert J M, Chari R V, Lutz R J, Wong W L, Jacobson F S, Koeppen H, Schwall R H, Kenkare-Mitra S R, Spencer S D, Sliwkowski M X. Targeting HER2-positive breast cancer with trastuzumab-DM1, an antibody-cytotoxic drug conjugate, Cancer Res 2008; 68:9280-90; Junttila T T, Li G, Parsons K, Phillips G L, Sliwkowski M X. Trastuzumab-DM1 (T-DM1) retains all the mechanisms of action of trastuzumab and efficiently inhibits growth of lapatinib insensitive breast cancer, Breast Cancer Res Treat (2010) 128(2):347-56; and Liu C and Chari R, The development of antibody delivery systems to target cancer with highly potent maytansinoids, Exp. Opin. Invest. Drugs (1997) 6(2):169-172.

New therapeutic strategies are critically needed to combat resistance and achieve more durable remissions:

### SUMMARY

The present invention is directed, in various embodiments, to combination therapies for treatment of cancer, including but not limited to hormone-resistant (hormone-refractory) breast cancers, such as those that are no longer responsive to first-line treatments such as administration to patients of aromatase inhibitors such as anastrozole, estrogen receptor modulators such as tamoxifen, and the like. The invention, in various embodiments, provides a method of treating a cancer, comprising administering to a patient afflicted therewith of an effective amount an immunoconjugate comprising a monoclonal antibody moiety and a first pro-apoptotic drug moiety linked thereto; and administering to the patient an effective amount of a second pro-apoptotic drug. For example, the cancer can be a breast cancer, such as an aromatase-resistant breast cancer, a tamoxifen-resistant breast cancer, an ER+ hormone refractory breast cancer, or a breast cancer comprising cancer cells in which HER2 expression is up-regulated, or any combination thereof.

In certain embodiments, the immunoconjugate comprises a monoclonal antibody moiety coupled via a linker with the first pro-apoptotic drug moiety. In various embodiments, the first pro-apoptotic drug moiety is a microtubule depolymerization agent, such as a maytansinoid or an auristatin. For example, the first pro-apoptotic drug moiety can be a maytansine analog (a maytansinoid), which is bonded via a linker moiety to the monoclonal antibody moiety. More specifically, the immunoconjugate can be a trastuzumab-maytansinoid conjugate, comprising trastuzumab (Herceptin®) coupled via a non-reducible linker moiety to a maytansinoid pro-apoptotic drug moiety (e.g., T-DM1). The inventors herein selected a pro-apoptotic strategy as preferable to a growth inhibition strategy to abrogate the process of adaptive reprogramming by eliminating the resistant cells rather than merely inhibiting their growth.

In various embodiments, the second pro-apoptotic drug exerts cytotoxicity by a molecular mechanism other than the molecular mechanism of cytotoxicity exerted by the first pro-apoptotic drug.

This is termed “horizontal modulation” herein, wherein two independent apoptotic pathways are activated or induced by the therapeutic regimen, as opposed to “vertical modulation”, wherein two or more steps in a single pro-apoptotic pathway are targeted. The inventors disclose herein that the horizontal modulation, employing, e.g., combinations of T-DM1 with a second pro-apoptotic drug, displayed synergistic effects in the induction of apoptosis in hormone refractory breast cancer cells.

In some embodiments, the second pro-apoptotic drug is a drug inducing apoptosis via an extrinsic pathway. In other embodiments, the second pro-apoptotic drug is a drug inducing apoptosis via an intrinsic pathway. For example, the second pro-apoptotic anticancer drug can be farnesyl-thiosalicylic acid (FTS), 4-(4-Chloro-2-methylphenoxy)-N-hydroxybutanamide (CMH), estradiol (E2), tetramethoxystilbene (TMS), δ-tocatrienol, salinomycin, or curcumin.

In various embodiments, the invention provides medical uses for a combination of a first pro-apoptotic drug and a second pro-apoptotic drug, for treatment of cancer, such as breast cancer, more specifically for treatment of a hormone-resistant breast cancer as described above. For example, the first pro-apoptotic drug can be an immunoconjugate such as T-DM1, and the second pro-apoptotic drug can be a drug that induces apoptosis in cancer cells by a molecular mechanism different from the molecular mechanism by which the first pro-apoptotic drug can exert its anticancer effects.

In various embodiments, the invention provides a therapeutic composition comprising an immunoconjugate comprising a monoclonal antibody moiety and a first pro-apoptotic drug moiety, and a second pro-apoptotic drug, for the treatment of cancer, such as breast cancer, more specifically for treatment of a hormone-resistant breast cancer as described above.

The monoclonal antibody moiety of the immunoconjugate can provide a targeting mechanism for the first pro-apoptotic drug, such as targeting up-regulated HER2 receptors in hormone-resistant breast cancer cells. A targeting component for an anticancer drug can be achieved by use of conjugates, e.g., covalently coupled moieties, one of which provides the targeting mechanism, the other of which provides the cytotoxic or apoptotic effect. One such agent, T-DM1, is a covalent conjugate of the monoclonal antibody trastuzumab (Herceptin®) with a maytansinoid macrocyclic pro-apoptotic cytotoxic agent. T-DM 1 comprises as a targeting moiety the HER2-specific humanized antibody trastuzumab covalently linked to the pro-apoptotic microtubule inhibitory agent DM1. See, for example, Oroudjev E, Lopus M, Wilson L, Audette C, Provenzano C, Erickson H, Kovtun Y, Chari R, Jordan M A (2010) Mol Cancer Ther 9:2700-2713, Maytansinoid-antibody conjugates induce mitotic arrest by suppressing microtubule polymerization. The inventors herein have surprisingly discovered that T-DM1, in combination with other pro-apoptoic anticancer drugs, including farnesyl-thiosalicylic acid (FTS, Salirasib, a Ras inhibitor that targets the intrinsic mitochondrial death pathway caspase dependent), estradiol (E2, intrinsic mitochondrial death pathway caspase dependent), tetramethoxystilbene (TMS, mitochondrial death pathway caspase independent), 4-(4-chloro-2-methylphenoxy)-N-hydroxybutanamide (CMH, extrinsic apoptotic pathway), δ-tocotrienol, salinomycin, or curcumin, or any combination thereof, can act synergistically to trigger non-toxic, apoptosis to kill hormone resistant (MCF-7; T47D) and hormone refractory (LTED; TamR) breast cancer cells in vitro. It is well known in the art that such cell lines used in evaluation of the therapies disclosed and claimed herein can be highly predictive of success for in vivo use of the therapy in patients suffering from cancer.

In various embodiments, the inventors herein disclose the results of experiments that were performed to confirm the hypothesis that combinations of certain pro-apoptotic agents can act synergistically to induce apoptosis, cell death, in hormone resistant (hormone refractory) breast cancer cells. For example, the invention provides a method of treatment of a cancer, comprising administration to a patient afflicted therewith of an effective amount of an immunoconjugate comprising a targeting monoclonal antibody and a first pro-apoptotic drug moiety, and administering to the patient an effective amount of a second pro-apoptotic drug. By synergistic is meant that the therapeutic effect is more than additive for the individual therapeutic effects that would be achieved by administration of each drug alone.

### BRIEF DESCRIPTION OF THE DRAWINGS

FIG. 1A-1F show electrophoresis gel autoradiograms (1A, 1B, 1D, 1E) and bar graphs (1C, 1F) summarizing results obtained thereby on levels of indicated pro-apoptotic proteins when LTED cells were treated with FTS, with examination of the changes in levels of the proteins in cytosolic and mitochondrial fractions.

FIG. 2A-B shows a time course bar graph (2A) and a cell viability versus concentration curve (2B) displaying (FIG. 2A) the effect of FTS and curcumin in combination on wild type MCF-7 cells; and (FIG. 2B) the effect of FTS alone or in combination with curcumin on MCF-7 cell viability.

FIG. 3A-B shows a time course bar graph (3A) and a cell viability versus concentration curve (3B) displaying the effect of salinomycin on MCF-7 cells.

FIG. 4 shows graphic illustrations of the dose effect plots of non-adopted cells: MCF-7 cells (a-c; upper graphs) and T47D (d-f; lower graphs) treated for five days with the combination indicated.

FIG. 5 shows graphic illustrations of the dose effect plots of adapted cell lines, LTED D29, treated for five days with the combination indicated.

FIG. 6 shows graphic illustrations of the combination index of non-adapted MCF-7 cells (a-c; upper three graphs) and T47D (d-f; lower three graphs) treated as indicated. Ordinate-Combination Index (CI); Abscissa-Fractional Effect

FIG. 7A, B shows graphic illustrations of the combination index of adapted cell lines, LTED D29 (a-i) and TamR cells (j-s) treated as indicated.

FIG. 8 shows graphical illustrations of an Isobologram analysis of non-adapted cells MCF-7 cells (a-c; upper three graphs) and T47D cells (d-f; lower three graphs) treated with the combination indicated. Ordinate-Dose A; Abscissa-Dose B.

FIG. 9 shows graphical illustrations of an Isobologram analysis of adapted cell lines, LTED D29 cells (a-i) treated as indicated. Ordinate-Dose A; Abscissa-Dose B.

### DETAILED DESCRIPTION

In describing and claiming the invention, the following terminology will be used in accordance with the definitions set forth below. Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described herein. As used herein, each of the following terms has the meaning associated with it in this section. Specific and preferred values listed below for radicals, substituents, and ranges are for illustration only; they do not exclude other defined values or other values within defined ranges for the radicals and substituents.

As used herein, the articles “a” and “an” refer to one or to more than one, i.e., to at least one, of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the term “affected cell” refers to a cell of a subject afflicted with a disease or disorder, which affected cell has an altered phenotype compared with a subject not afflicted with a disease, condition, or disorder.

Cells or tissue are “affected” by a disease or disorder if the cells or tissue have an altered phenotype relative to the same cells or tissue in a subject not afflicted with a disease, condition, or disorder.

As used herein, an “agonist” is a composition of matter that, when administered to a mammal such as a human, enhances or extends a biological activity of interest. Such effect may be direct or indirect.

An “antagonist” is a composition of matter that when administered to a mammal such as a human, inhibits or impedes a biological activity attributable to the level or presence of an endogenous compound in the mammal. Such effect may be direct or indirect.

As used herein, the term “aromatase inhibitor” relates to a composition that blocks the conversion of androstenedione to estrone and/or testosterone to estradiol. Aromatase inhibitors include both steroidal and nonsteroidal classes of inhibitors including for example, exemestane, anastrozole and letrozole.

As used herein, an “analog” of a chemical compound is a compound that, by way of example, resembles another in structure but is not necessarily an isomer (e.g., 5-fluorouracil is an analog of thymine).

The term “apoptosis” refers to programmed cell death mediated by biochemical pathways that can be induced by various means. A “pro-apoptotic” agent or drug is a bioactive agent or drug that produces a biochemical effect that results in programmed cell death. As described herein, apoptosis can be caused or induced by intrinsic or extrinsic pathways or mechanisms, as further described below. The “extrinisic” apoptosis pathway involves death receptors, and this pathway is activated by ligands that bind to the death receptors. The “intrinsic” apoptosis pathway involves mitochondrial pathways that initiate apoptosis. “Horizontal” as in horizontal modulation refers to stimuli that affect more than one specific pathway whereas “vertical” as in vertical modulation means that several steps in the same pathway re involved.

As used herein, the term “breast cancer” relates to any of various types and subtypes of carcinomas of the breast or mammary tissue.

The term “cancer” as used herein is defined as proliferation of cells whose unique trait—loss of normal controls—results in unregulated growth, lack of differentiation, local tissue invasion, and metastasis. Examples include but are not limited to, breast cancer, prostate cancer, ovarian cancer, cervical cancer, skin cancer, pancreatic cancer, colorectal cancer, renal cancer and lung cancer.

A “compound,” as used herein, refers to any type of substance or agent that is commonly considered a drug, or a candidate for use as a drug, as well as combinations and mixtures of the above.

A “conjugate” is a molecular entity combining at least two “moieties”, or domains, in association with each other. A “covalent” conjugate is a conjugate wherein the moieties are associated by means of covalent chemical bonds, such as are well-known in the art. For example, a protein, such as a monoclonal antibody, can be caused to form a conjugate with an organic compound such as a drug, such as through covalent bonding. When the protein is an antibody, e.g., a monoclonal antibody, the resulting conjugate is referred to herein as an “immunoconjugate.” Covalent bonding between a protein (e.g., a monoclonal antibody) and an organic compound (e.g., a drug) can take place through a “linker” or “linker moiety”, which is covalently bonded both to the organic compound and to the protein. Examples are discussed below.

As used herein, the term “linker” or “linker moiety” refers to a molecular moiety that joins two other molecular moieties either covalently, or noncovalently, e.g., through ionic or hydrogen bonds or van der Waals interactions. Specific examples are provided below. “Linkage” or “linker” refers to a connection between two groups.

A “moiety” as the term is used herein refers to a domain of a larger molecule; for example, in the conjugate T-DM1, the maytansinoid drug is coupled via a linker to the monoclonal antibody, such that in the final product a maytansinoid drug moiety is bonded via a linker moiety to a monoclonal antibody moiety. An example of a conjugate comprising such moieties is provided by the molecular entity T-DM1, which is a covalent conjugate of the monoclonal antibody trastumuzab (Herceptin®), and a maytansinoid macrocyclic cytotoxic compound, the structure of which is shown below:

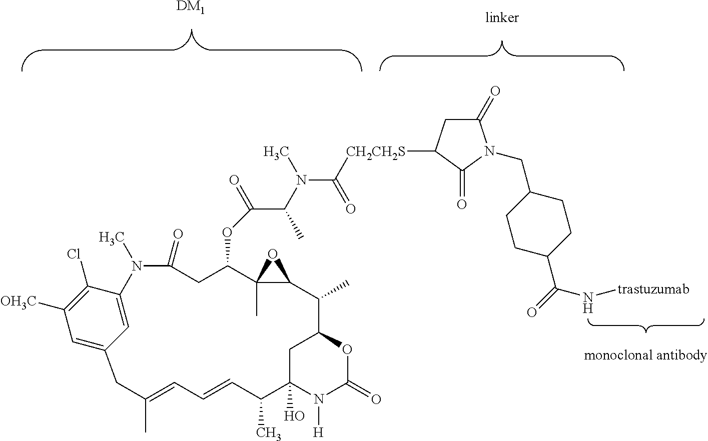

It is believed that the coupling of the linker to the trastuzumab is via bonding of the linker moiety to the nitrogen atom of a sidechain aminoacid residue of the protein trastuzumab, such as a lysine residue. The molecular structure of trastuzumab, being well-known in the art, is not provided in detail. Immunoconjugates, such as of an antibody and a pro-apoptotic drug, such as a maytansinoid, can be prepared and evaluated by methods described herein and in documents incorporated by reference herein.

An immunoconjugate comprises an antibody conjugated to one or more bioactive molecules. The immunoconjugate T-DM1, as shown above, comprises a maytansinoid moiety coupled to the monoclonal antibody moiety. Maytansinoids are mitototic inhibitors which act by inhibiting tubulin polymerization or inducing microtubule depolymerization. As is well known in the art, tubulin polymerization and depolymerization are essential events involved in mitosis, cell division. Prolonged suppression of cell division is believed to be a state that can induce apoptosis in the mitosis-suppressed cell.

Maytansine was first isolated from the east African shrub Maytenus serrata (U.S. Pat. No. 3,896,111). Subsequently, it was discovered, that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533.

Maytansinoid drug moieties are attractive drug moieties in antibody-drug conjugates because they are: (i) relatively accessible to prepare by fermentation or chemical modification or derivatization of fermentation products, (ii) amenable to derivatization with functional groups suitable for conjugation through non-disulfide (non-reducible) linkers to antibodies, (iii) stable in plasma, and (iv) effective against a variety of tumor cell lines.

Maytansine compounds suitable for use as maytansinoid drug moieties are well known in the art and can be isolated from natural sources according to known methods or produced using genetic engineering techniques (see Yu et al (2002) PNAS 99:7968-7973). Maytansinol and maytansinol analogues may also be prepared synthetically according to known methods.

Maytansinoid drug moieties include those having a modified aromatic ring, such as: C-19-dechloro (U.S. Pat. No. 4,256,746) (prepared by lithium aluminum hydride reduction of ansamytocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/−dechloro (U.S. Pat. No. 4,294,757) (prepared by acylation using acyl chlorides), and those having modifications at other positions.

Exemplary maytansinoid drug moieties also include those having modifications such as: C-9-SH (U.S. Pat. No. 4,424,219) (prepared by the reaction of maytansinol with H₂S or P₂S₅); C-14-alkoxymethyl(demethoxy/CH₂OR) (U.S. Pat. No. 4,331,598); C-14-hydroxymethyl or acyloxymethyl (CH₂OH or CH₂OAc) (U.S. Pat. No. 4,450,254) (prepared from Nocardia); C-15-hydroxy/acyloxy (U.S. Pat. No. 4,364,866) (prepared by the conversion of maytansinol by Streptomyces); C-15-methoxy (U.S. Pat. Nos. 4,313,946 and 4,315,929) (isolated from Trewia nudlflora); C-18-N-demethyl (U.S. Pat. Nos. 4,362,663 and 4,322,348) (prepared by the demethylation of maytansinol by Streptomyces); and 4,5-deoxy (U.S. Pat. No. 4,371,533) (prepared by the titanium trichloride/LAH reduction of maytansinol).

An “auristatin”, as the term is used herein refers to peptidic anticancer drugs such as the dolastatins and auristatins that have been shown to interfere with microtubule dynamics, GTP hydrolysis, and nuclear and cellular division (Woyke et al (2001) Antimicrob. Agents and Chemother 45(12):3580-3584) and have anticancer (U.S. Pat. No. 5,663,149) and antifungal activity (Pettit et al (1998) Antimicrob. Agents Chemother 42:2961-2965). See for example U.S. Pat. Nos. 5,635,483 and 5,780,588.

A “control” subject is a subject having the same characteristics as a test subject, such as a similar type of dependence, etc. The control subject may, for example, be examined at precisely or nearly the same time the test subject is being treated or examined. The control subject may also, for example, be examined at a time distant from the time at which the test subject is examined, and the results of the examination of the control subject may be recorded so that the recorded results may be compared with results obtained by examination of a test subject.

A “test” subject is a subject being treated.

As used herein, a “derivative” of a compound refers to a chemical compound that may be produced from another compound of similar structure in one or more steps, as in replacement of H by an alkyl, acyl, or amino group.

A “disease” is a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder.

A disease, condition, or disorder is “alleviated” if the severity of a symptom of the disease or disorder, the frequency with which such a symptom is experienced by a patient, or both, are reduced.

As used herein, an “effective amount” means an amount sufficient to produce a selected effect, such as alleviating symptoms of a disease or disorder. In the context of administering two or more compounds, the amount of each compound, when administered in combination with another compound(s), may be different from when that compound is administered alone.

As used herein, the term “estrogen” relates to a class of compounds including naturally occurring and synthetically made compositions that have a demonstrated ability to induce cell proliferation and/or initiate new protein synthesis in estrogen responsive cells. Naturally occurring estrogens include estrone (E1), estradiol-17B (E2), and estriol (E3), and of these, estradiol is the most active pharmacologically. Synthetic estrogens are compounds that do not occur in nature and duplicate or mimic the activity of endogenous estrogens in some degree. These compounds include a variety of steroidal and non-steroidal compositions exemplified by dienestrol, benzestrol, hexestrol, methestrol, diethylstilbestrol (DES), quinestrol (Estrovis), chlorotrianisene (Tace), and methallenestril (Vallestril).

As used herein, the term “estrogen antagonist” relates to a compound that has a neutralizing or inhibitory effect on an estrogen's activity when administered simultaneously with that estrogen. Examples of estrogen inhibitors include tamoxifen and toremifene.

As used herein, a “functional” molecule is a molecule in a form in which it exhibits a property or activity by which it is characterized. A functional enzyme, for example, is one that exhibits the characteristic catalytic activity by which the enzyme is characterized.

As used herein, the term “hormone deprivation therapy” relates to any treatment of a patient that blocks the action of, or removes (either by preventing synthesis or enhancing the destruction of the hormone) the presence of hormones, from a patient. In the specific case of a breast cancer, hormone deprivation therapy can include deprivation of estrogen, by blocking the biosynthesis of estrogen, or blocking the effect of estrogen on an estrogen receptor such as HER2.

As used herein, the term “hormone responsive cells/tissue” relates to non-cancerous cells or tissues that are naturally responsive to, e.g., estrogens or androgens, wherein the cells or tissue proliferate and/or initiate new protein synthesis in the presence of the hormone. Hormone responsive tissues include the mammary glands, testes, prostate, uterus and cervix. A tissue which is normally responsive to estrogens or androgens may lose its responsiveness to the hormone. Thus, “hormone responsive tissue” is a broad term as used herein and encompasses both hormone-sensitive and hormone insensitive tissues that are normally responsive to hormones. An “estrogen responsive cell/tissue” is one that is responsive to estrogen.

As used herein, the term “hormone responsive cancers” relates to cells or tissues that are derived from hormone responsive cells/tissue, and an “adapted hormone responsive cancer cell” is a hormone responsive cancer cell that will proliferate in response to levels of hormone that would not produce a response in a corresponding hormone responsive cell.

Upon exposure to substances such as aromatase inhibitors that block estrogen production as described above, or estrogen antagonists such as tamoxifen, or other agents of similar estrogen-blocking effect, estrogen-responsive cancer cells such as are found in breast cancer can develop resistance to decrease of estrogen levels in the tissue. In such cases, use of the aromatase inhibitors is no longer effective in controlling the breast cancer. The cells involved in such cancers are herein termed “long-term estrogen deprived”, “hormone-resistant”, or “hormone-refractory” cells, and the macroscopic disease is termed, interchangeably, “hormone-resistant” or “hormone-refractory” breast cancer. However, it is understood that “hormone-resistant”, or “hormone-refractory” cells and cancers can arise via other mechanisms as well.

As used herein, the term “adapted hormone response” or “adapted response” relates to the process by which cells or tissues that are derived from hormone responsive tissue become able to respond to (i.e. proliferate and/or initiate new protein synthesis) levels of hormone that previously would not produce a response in those cells.

The term “inhibit,” as used herein, refers to the ability of a compound or any agent to reduce or impede a described function, level, activity, synthesis, release, binding, etc., based on the context in which the term “inhibit” is used. Preferably, inhibition is by at least 10%, more preferably by at least 25%, even more preferably by at least 50%, and most preferably, the function is inhibited by at least 75%. The term “inhibit” is used interchangeably with “reduce” and “block”.

The term “inhibit a protein”, as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time. An “inhibitor” can carry out any of these functions, e.g., an aromatase inhibitor blocks the biosynthetic catalytic activity whereby the aromatase enzyme (a protein) converts a precursor to an estrogen.

As used herein, the term “inhibition of mTOR activity” relates to a detectable decrease in mTOR's ability to phosphorylate one or more of its substrates including, for example, p70 S6K and PHAS-I. An mTOR inhibitor is a compound that has a direct inhibitory effect on mTOR activity (i.e. the inhibition of mTOR activity is not mediated though an inhibitory effect on an upstream pathway enzyme).

As used herein, an “instructional material” includes a publication, a recording, a diagram, or any other medium of expression which can be used to communicate the usefulness of a compound of the invention in the kit for effecting alleviation of the various diseases or disorders recited herein. Optionally, or alternately, the instructional material may describe one or more methods of alleviating the diseases or disorders in a subject. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

A “receptor” is a compound or molecule that specifically binds to a ligand.

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone.

The term “peptide” typically refers to short polypeptides.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

A “recombinant polypeptide” is one which is produced upon expression of a recombinant polynucleotide.

The term “per application” as used herein refers to administration of a drug or compound to a subject.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans. A “pharmaceutically acceptable salt” refers to a molecular entity, either acidic or basic, in the form of a salt with a counterion that is pharmaceutically acceptable in terms of approval by a regulatory agency or listing in the US Pharmacopeia.

As used herein, the term “physiologically acceptable” ester or salt means an ester or salt form of the active ingredient which is compatible with any other ingredients of the pharmaceutical composition, and which is not deleterious to the subject to which the composition is to be administered.

The term “prevent”, as used herein, means to stop something from happening, or taking advance measures against something possible or probable from happening. In the context of medicine “prevention” generally refers to action taken to decrease the chance of getting a disease or condition.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

The term “regulate” refers to either stimulating or inhibiting a function or activity of interest.

A “sample,” as used herein, refers to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest.

By the term “specifically binds,” as used herein, is meant a molecule which recognizes and binds a specific molecule, but does not substantially recognize or bind other molecules in a sample, or it means binding between two or more molecules as in part of a cellular regulatory process, where said molecules do not substantially recognize or bind other molecules in a sample.

The term “standard,” as used herein, refers to something used for comparison. For example, it can be a known standard agent or compound which is administered or added and used for comparing results when adding a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of diagnosis or treatment is a mammal, including a human.

The term “symptom,” as used herein, refers to any morbid phenomenon or departure from the normal in structure, function, or sensation, experienced by the patient and indicative of disease. In contrast, a sign is objective evidence of disease. For example, a bloody nose is a sign. It is evident to the patient, doctor, nurse and other observers.

As used herein, the term “treating” may include prophylaxis of the specific disease, disorder, or condition, or alleviation of the symptoms associated with a specific disease, disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease. “Treating” is used interchangeably with “treatment” herein. For example, treating cancer includes preventing or slowing the growth and/or division of cancer cells as well as killing cancer cells or reducing the size of a tumor. Additional signs of successful treatment of cancer include normalization of tests such as white blood cell count, red blood cell count, platelet count, erythrocyte sedimentation rate, and various enzyme levels such as transaminases and hydrogenases. Additionally, the clinician may observe a decrease in a detectable tumor marker such as prostatic specific antigen (PSA).

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

In various embodiments of a method of the invention, the immunoconjugate can be any of the immunoconjugates discussed above. The structure of T-DM1 is provided above.

Pro-apoptotic drugs mentioned herein include:

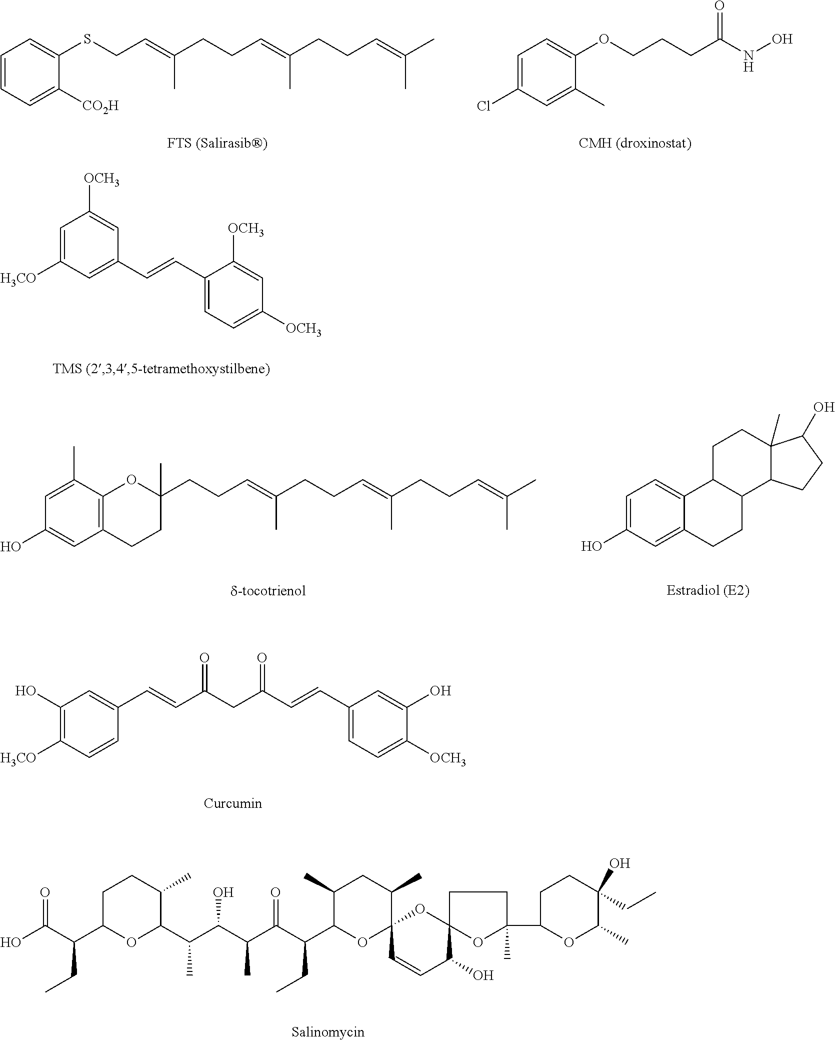

### EMBODIMENTS

The present invention is directed, in various embodiments, to development of anticancer therapies suitable for disease states where development of resistance of the cancer cells has occurred or has a high probability of occurring, using an additive or synergistic combination of agents that induce cell death (apoptosis), as disclosed and claimed herein. By use of these methods, the development of resistance to the drugs by the cancer cells can be diminished relative to the frequency of resistance development using a single anticancer drug, or resistance can be overcome in cancer cells that have already undergone adaptive mutation to a therapy. In various embodiments, the inventive methods can be effective for treatment of hormone-resistant breast cancer, where the cancer is no longer responsive to first-line treatments such as the use of aromatase inhibitors such as anastrazole and the like, or the use of estrogen receptor modulators or antagonists such as tamoxifen and the like.

For example, the therapy-resistant cancer can be a breast cancer, such as an aromatase-resistant breast cancer, a tamoxifen-resistant breast cancer, a ER+ hormone refractory breast cancer, or a breast cancer comprising cancer cells in which HER2 expression is up-regulated (HER2-positive breast cancer), or any combination thereof.

As discussed above, long term estrogen deprived cells, such as estrogen-responsive breast cancer cells in patients who have received aromatase inhibitor or estrogen antagonist therapy, can develop an increased degree of responsiveness to estrogen levels well below those normally found in breast tissue. This effect can occur as a result of up-regulation and overexpression of the genes encoding HER receptors, such as the genes encoding HER2. In a breast cancer patient who has been treated with first-line agents such as aromatase inhibitors or estrogen antagonists, such cells can proliferate in the absence of normal estrogen levels, resulting in a breast cancer that has developed resistance to these first-line therapies, i.e., has become a hormone-resistant or hormone-refractory breast cancer. In such disease states, use of a second-line therapy becomes vital for patient survival. In various embodiments, the present invention provides a second-line therapy for treatment of hormone-resistant breast cancers, such as those cancers that have developed resistance by such a mechanism.

A pro-apoptotic strategy was chosen by the inventors herein as preferable to a growth inhibition strategy to reduce the frequency of adaptive mutations in the target cells by elimination of the resistant cancer cells, rather than merely inhibiting their growth. The inventors herein also disclose that the use of at least two pro-apoptotic agents acting horizontally, i.e., on two different pathways (“horizontal modulation”), each of which can result in induction of apoptosis and cell death, has been found to provide an additive or synergistic effect in induction of apoptosis wherein the frequency of resistance development is diminished. In second-line therapies involving treatment of hormone-resistant breast cancer cells, the pro-apoptotic strategy can reduce the probability of further adaptive mutations, by inducing cell death. The further use of horizontal modulation can serve to further reduce the probability of cells avoiding apoptosis by adaptive mutations, as more than a single apoptosis-inducing mechanism can be invoked, and the probabilities of two mechanisms of resistance developing in a single cell prior to its death is lower than the probability of a single mechanism of resistance developing.

Apoptosis can occur either through death receptor pathways (extrinsic pathways) or by mitochondrial-mediated pathways (intrinsic pathways). The inventors herein have unexpectedly discovered additive and synergistic combinations of pro-apoptotic anticancer agents, such as a pair of pro-apoptotic agents acting to induce apoptosis via distinct molecular mechanisms, that are effective in killing hormone-adapted cancer cells in well-characterized cells lines, such as Tamoxifen resistant (TamR) and long-term estrogen deprived (LTED) cell lines for models of hormone-refractory breast cancer, and for comparison, wild type MCF-7 and T47D cell lines. It is believed that synergistic effects are most likely to occur when two pro-apoptotic anticancer agents induce apoptosis by different mechanisms.

In various embodiments, a method of treatment of the invention provides synergistic therapeutic effects in cancer treatment, especially breast cancer. The present application discloses the surprising results that certain combinations of drugs provide a synergistic effect when treating breast cancer cells. In an embodiment, a combination treatment using FTS and CMH is found to provide synergistic effects. In an embodiment, a combination treatment using TMS and CMH provides synergistic effects. Therefore, the present invention further encompasses combination therapies using not just FTS, TMS, and CMH, but also drugs with similar activities, administration of two or more such drugs in conjunction can provide synergistic therapeutic effects, such as in the treatment of a breast cancer, e.g., hormone-refractory and hormone-resistant breast cancers. For example, combinations using FTS and ES provide surprisingly high synergistic effects.

In some embodiments, the combination of T-DM1 with any of E2, FTS and CMH provides synergistic effects, with the combination of T-DM1 with either FTS or CMH showing the strongest synergy, see Table 1, below.

The inventors herein disclose methods of treatment comprising administration to a patient afflicted with cancer, such as breast cancer, two or more pro-apoptotic anticancer agent that affect cells through horizontal modulation, wherein the two agents act on distinct apoptotic pathways, rather than on sequential steps in a single apoptotic pathway (vertical modulation). The extrinisic pathway involves death receptors and this pathway is activated by ligands that bind to the death receptors. The intrinsic pathway involves mitochondrial pathways that initiate apoptosis. Horizontal refers to stimuli that affect more than one specific pathway whereas vertical means that several steps in the same pathway re involved. In various embodiments, the at least two drugs achieve horizontal modulation. In various embodiments, horizontal modulation provides synergistic effects of the at least two drugs. In various embodiments, combination therapy of the invention using at least two drugs produces synergistic effects on non-adapted breast cancer cells.

A method of the invention, in various embodiments, provides a method of treating a cancer, comprising administering to a patient afflicted therewith of an effective amount an immunoconjugate comprising a monoclonal antibody moiety and a first pro-apoptotic drug moiety linked thereto via a linker moiety; and administering to the patient an effective amount of a second pro-apoptotic drug. For example, the immunoconjugate can comprise a first pro-apoptotic drug moiety covalently linked thereto, as discussed in greater detail below.

For example, the cancer to be treated can be a breast cancer. More-specifically, the breast cancer can be a hormone-resistant (hormone-refractory) breast cancer, such as results from proliferation of long-term estrogen deprived cells that have undergone adaptive mutation. In some adaptive mutations conferring hormone resistance, the breast cancer is referred to as HER2 resistant breast cancer. By a HER2 resistant breast cancer is meant a breast cancer wherein the cells have undergone adaptive mutation providing resistance to first-line treatments that target molecular entities and their interactions with the HER2 receptor. In some adaptive mutations conferring hormone resistance, the breast cancer is referred to as aromatase resistant breast cancer. By aromatase resistant breast cancer is meant a breast cancer that has become resistant to aromatase therapy. As described above, aromatase is an enzyme involved in a key step of estrogen biosynthesis.

In various embodiments, the targeting moiety of the immunoconjugate binds to the HER2 receptor. In cancer cells that have become resistant to aromatase or estrogen antagonist therapy, overexpression of the HER2 receptor can be a cause. When overexpression has taken place, the receptor becomes selectively more abundant per cell in the resistant cancer cells, and in the presence of a HER2 specific monoclonal antibody targeting moiety, can bind more molecules per cell of the antibody-drug conjugate. With the immunoconjugate localized on the tumor cell, a higher local concentration of the first pro-apoptotic anticancer drug moiety can be achieved. See Liu C and Chari R, The development of antibody delivery systems to target cancer with highly potent maytansinoids, Exp. Opin. Invest. Drugs (1997) 6(2):169-172.

Accordingly, in various embodiments, the inventive method can be used in the treatment of a cancer, wherein the cancer is breast cancer. More specifically, the breast cancer can be an aromatase-resistant breast cancer; or the breast cancer can be ER+ hormone refractory breast cancer; or the breast cancer can be HER2 positive breast cancer; or the breast cancer comprises cancer cells in which HER2 expression is up-regulated. In various embodiments, the immunoconjugate as described above binds to receptor HER2 as expressed in breast cancer cells, such as in hormone-resistant breast cancer cells. For example the monoclonal antibody of the immunoconjugate can be trastuzumab, which is known to be specific for the HER2 receptor.

In various embodiments, the first pro-apoptotic anticancer drug moiety can be a microtubule depolymerization agent, such as a maytansinoid or an auristatin. For example the covalent conjugate can consist essentially of trastuzumab covalently coupled via a linker with a maytansinoid pro-apoptotic anticancer drug moiety.

Exemplary embodiments of maytansinoid drug moieties that can be conjugated with a monoclonal antibody targeting moiety include: DM1; DM3; and DM4, having the structures:

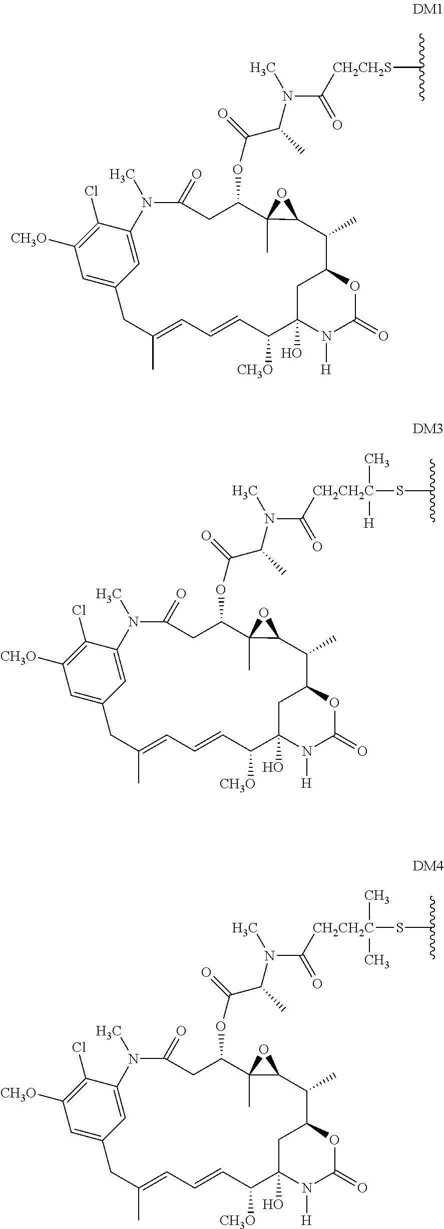

wherein the wavy line indicates the covalent attachment of the sulfur atom of the drug to a linker (L) of an antibody-drug conjugate. HERCEPTIN® (trastuzumab) linked by SMCC to DM1 has been reported (WO 2005/037992; US 2005/0276812 A1).

In various embodiments the covalent immunoconjugate is T-DM1 (structure shown above), that is, DM1 as shown above, covalently coupled to trastuzumab. In other embodiments, the covalent immunoconjugate can be another maytansinoid such as DM3 or DM4 coupled to trastuzumab. For example, maytansinoid antibody-drug conjugates used in practice of the inventive method can have the following structures and abbreviations, (wherein Ab is antibody and p is 1 to about 8):

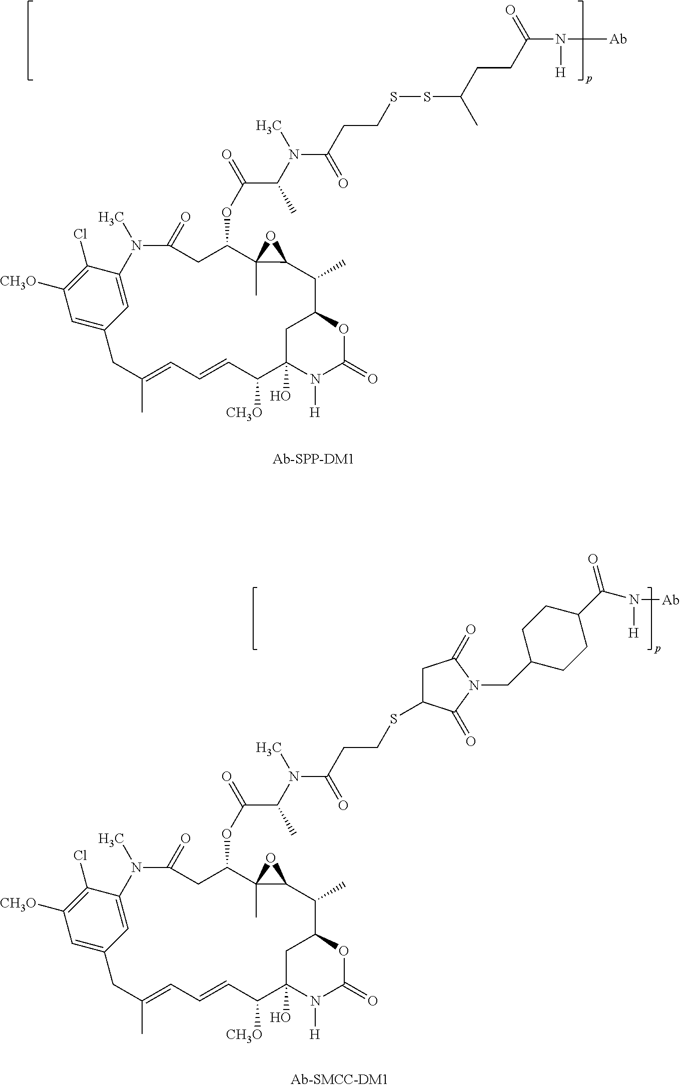

Exemplary antibody-drug conjugates where DM1 is linked through a BMPEO linker to a thiol group of the antibody have the structure and abbreviation:

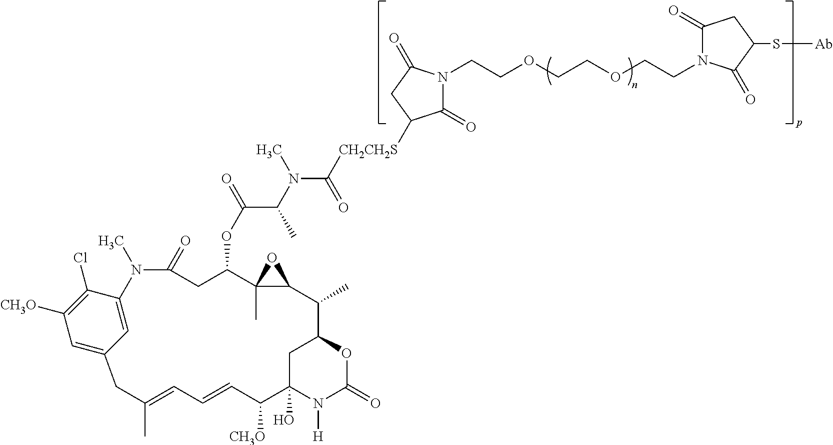

where Ab is antibody; n is 0, 1, or 2; and p is 1, 2, 3, or 4.

Immunoconjugates containing maytansinoids, methods of making the same, and their therapeutic use are disclosed, for example, in U.S. Pat. Nos. 5,208,020, 5,416,064, US 2005/0276812 A1, and European Patent EP 0 425 235 B1, the disclosures of which are hereby expressly incorporated by reference. Liu et al. Proc. Natl. Acad. Sci. USA 93:8618-8623 (1996) describe immunoconjugates comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay. Chari et al. Cancer Research 52:127-131 (1992) describe immunoconjugates in which a maytansinoid was conjugated via a disulfide linker to the murine antibody A7 binding to an antigen on human colon cancer cell lines, or to another murine monoclonal antibody TA.1 that binds the HER2/neu oncogene. The cytotoxicity of the TA.1-maytansonoid conjugate was tested in vitro on the human breast cancer cell line SK-BR-3, which expresses 3×10⁵ HER2 surface antigens per cell. The drug conjugate achieved a degree of cytotoxicity similar to the free maytansinoid drug, which could be increased by increasing the number of maytansinoid molecules per antibody molecule. The A7-maytansinoid conjugate showed low systemic cytotoxicity in mice.

Antibody-maytansinoid conjugates are prepared by chemically linking an antibody to a maytansinoid molecule without significantly diminishing the biological activity of either the antibody or the maytansinoid molecule. See, e.g., U.S. Pat. No. 5,208,020 (the disclosure of which is hereby expressly incorporated by reference). An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pat. No. 5,208,020 and in the other patents and nonpatent publications referred to hereinabove. Preferred maytansinoids are maytansinol and maytansinol analogues modified in the aromatic ring or at other positions of the maytansinol molecule, such as various maytansinol esters.

There are many linking groups known in the art for making antibody-maytansinoid conjugates, including, for example, those disclosed in U.S. Pat. No. 5,208,020 or EP Patent 0 425 235 B1; Chari et al. Cancer Research 52:127-131 (1992); and US 2005/016993 A 1, the disclosures of which are hereby expressly incorporated by reference. Antibody-maytansinoid conjugates comprising the linker component SMCC may be prepared as disclosed in US 2005/0276812 A1, “Antibody-drug conjugates and Methods.” The linkers comprise disulfide groups, thioether groups, acid labile groups, photolabile groups, peptidase labile groups, or esterase labile groups, as disclosed in the above-identified patents. Additional linkers are described and exemplified herein.

In various embodiments, the covalent conjugate comprises a linker moiety that is selected such that after entry into the body, the linkage is broken, such as by enzymatic action, acid hydrolysis, base hydrolysis, or the like, and the two separate compounds are then formed. In other embodiments, the linker moiety is selected for stability under biological conditions, wherein the pro-apoptotic anticancer drug moiety can exert a cytotoxic effect while still tethered to the targeting moiety, such as a monoclonal antibody like trastuzumab.

Data from previous structure-activity relationship (SAR) studies within the art may be used as a guide to determine which compounds to use and the optimal position or positions on the molecules to attach the tether such that potency and selectivity of the compounds will remain high. The tether or linker moiety is chosen from among those of demonstrated utility for linking bioactive molecules together. Disclosed herein are representative compounds that can be attached together in different combinations to form heterobivalent therapeutic molecules.

Conjugates of the antibody and maytansinoid may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). In certain embodiments, the coupling agent is N-succinimidyl-3-(2-pyridyldithio) propionate (SPDP) (Carlsson et al., Biochem. J. 173:723-737 (1978)) or N-succinimidyl-4-(2-pyridylthio)pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hydroxymethyl, the C-15 position modified with a hydroxyl group, and the C-20 position having a hydroxyl group. In one embodiment, the linkage is formed at the C-3 position of maytansinol or a maytansinol analogue.

An immunoconjugate, such as can be used in various embodiments of the methods of treatment and uses of the present invention, can comprise a targeting monoclonal antibody conjugated to a microtubule depolymerization agent, such as a maytansinoid, as described above, or can comprise as a first pro-apoptotic anticancer drug moiety a dolastatin or a dolastatin peptidic analog or derivative, e.g., an auristatin (see U.S. Pat. Nos. 5,635,483; 5,780,588). Dolastatins and auristatins have been shown to interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). The dolastatin or auristatin drug moiety may be attached to the antibody through the N (amino) terminus or the C (carboxyl) terminus of the peptidic drug moiety (WO 02/088172).

Exemplary auristatin embodiments include the N-terminus linked monomethylauristatin drug moieties D_{E} and D_{F}, disclosed in Senter et al, Proceedings of the American Association for Cancer Research, Volume 45, Abstract Number 623, presented Mar. 28, 2004, the disclosure of which is expressly incorporated by reference in its entirety. Examples are shown below.

A auristatin-analogous pro-apoptotic anticancer drug moiety may be selected from Formulas D_{E} and D_{F} below:

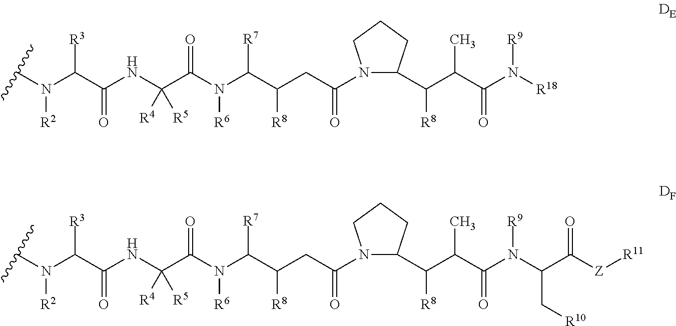

wherein the wavy line of D_{E} and D_{F} indicates the covalent attachment site to an antibody or antibody-linker component, and independently at each location:

R² is selected from H and C₁-C₈ alkyl;

R³ is selected from H, C₁-C₈ alkyl, C₃-C₈ carbocycle, aryl, C₁-C₈ alkyl-aryl, C₁-C₈ alkyl-(C₃-C₈ carbocycle), C₃-C₈ heterocycle and C₁-C₈ alkyl-(C₃-C₈ heterocycle);

R⁴ is selected from H, C₁-C₈ alkyl, C₃-C₈ carbocycle, aryl, C₁-C₈ alkyl-aryl, C₁-C₈ alkyl-(C₃-C₈ carbocycle), C₃-C₈ heterocycle and C₁-C₈ alkyl-(C₃-C₈ heterocycle);

R⁵ is selected from H and methyl;

or R⁴ and R⁵ jointly form a carbocyclic ring and have the formula —(CR^{a}R^{b})ₙ— wherein R^{a} and R^{b} are independently selected from H, C₁-C₈ alkyl and C₃-C₈ carbocycle and n is selected from 2, 3, 4, 5 and 6;

R⁶ is selected from H and C₁-C₈ alkyl;

R⁷ is selected from H, C₁-C₈ alkyl, C₃-C₈ carbocycle, aryl, C₁-C₈ alkyl-aryl, C₁-C₈ alkyl-(C₃-C₈ carbocycle), C₃-C₈ heterocycle and C₁-C₈ alkyl-(C₃-C₈ heterocycle);

each R⁸ is independently selected from H, OH, C₁-C₈ alkyl, C₃-C₈ carbocycle and O—(C₁-C₈ alkyl);

R⁹ is selected from H and C₁-C₈ alkyl;

R¹⁰ is selected from aryl or C₃-C₈ heterocycle;

Z is O, S, NH, or NR¹², wherein R¹² is C₁-C₈ alkyl;

R¹¹ is selected from H, C₁-C₂₀ alkyl, aryl, C₃-C₈ heterocycle, —(R¹³O)ₘ—R¹⁴, or —(R¹³)ₘ—CH(R¹⁵)₂;

m is an integer ranging from 1-1000;

R¹³ is C₂-C₈ alkyl;

R¹⁴ is H or C₁-C₈ alkyl;

each occurrence of R¹⁵ is independently H, COOH, —(CH₂)ₙ—N(R¹⁶)₂, —(CH₂)ₙ—SO₃H, or —(CH₂)ₙ—SO₃—C₁-C₈ alkyl;

each occurrence of R¹⁶ is independently H, C₁-C₈ alkyl, or —(CH₂)ₙ—COOH;

R¹⁸ is selected from —C(R⁸)₂—C(R⁸)₂-aryl, —C(R⁸)₂—C(R⁸)₂—(C₃-C₈ heterocycle), and —C(R⁸)₂—C(R⁸)₂—(C₃-C₈ carbocycle); and

n is an integer ranging from 0 to 6.

In one embodiment, R³, R⁴ and R⁷ are independently isopropyl or sec-butyl and R⁵ is —H or methyl. In an exemplary embodiment, R³ and R⁴ are each isopropyl, R⁵ is —H, and R⁷ is sec-butyl.

In yet another embodiment, R² and R⁶ are each methyl, and R⁹ is —H.

In still another embodiment, each occurrence of R⁸ is —OCH₃.

In an exemplary embodiment, R³ and R⁴ are each isopropyl, R² and R⁶ are each methyl, R⁵ is —H, R⁷ is sec-butyl, each occurrence of R⁸ is —OCH₃, and R⁹ is —H.

In one embodiment, Z is —O— or —NH—.

In one embodiment, R¹⁰ is aryl.

In an exemplary embodiment, R¹⁰ is -phenyl.

In an exemplary embodiment, when Z is —O—, R¹¹ is —H, methyl or t-butyl.

In one embodiment, when Z is —NH, R¹¹ is —CH(R¹⁵)₂, wherein R¹⁵ is —(CH₂)ₙ—N(R¹⁶)₂, and R¹⁶ is —C₁-C₈ alkyl or —(CH₂)ₙ—COOH.

In another embodiment, when Z is —NH, R¹¹ is —CH(R¹⁵)₂, wherein R¹⁵ is —(CH₂)ₙ—SO₃H.

An exemplary auristatin embodiment of formula D_{E} is MMAE, wherein the wavy line indicates the covalent attachment to a linker (L) of an antibody-drug conjugate:

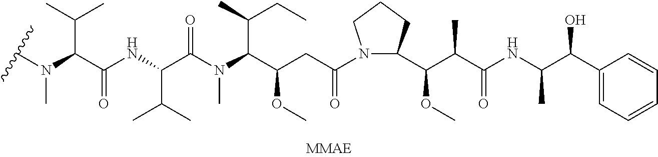 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

An exemplary auristatin embodiment of formula D_{F} is MMAF, wherein the wavy line indicates the covalent attachment to a linker (L) of an antibody-drug conjugate (see US 2005/0238649 and Doronina et al. (2006) Bioconjugate Chem. 17:114-124):

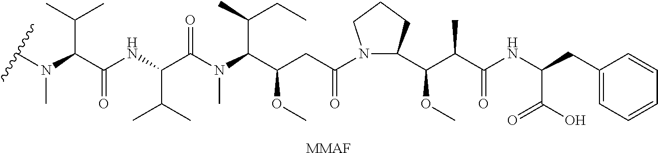

Other drug moieties include the following MMAF derivatives, wherein the wavy line indicates the covalent attachment to a linker (L) of an antibody-drug conjugate:

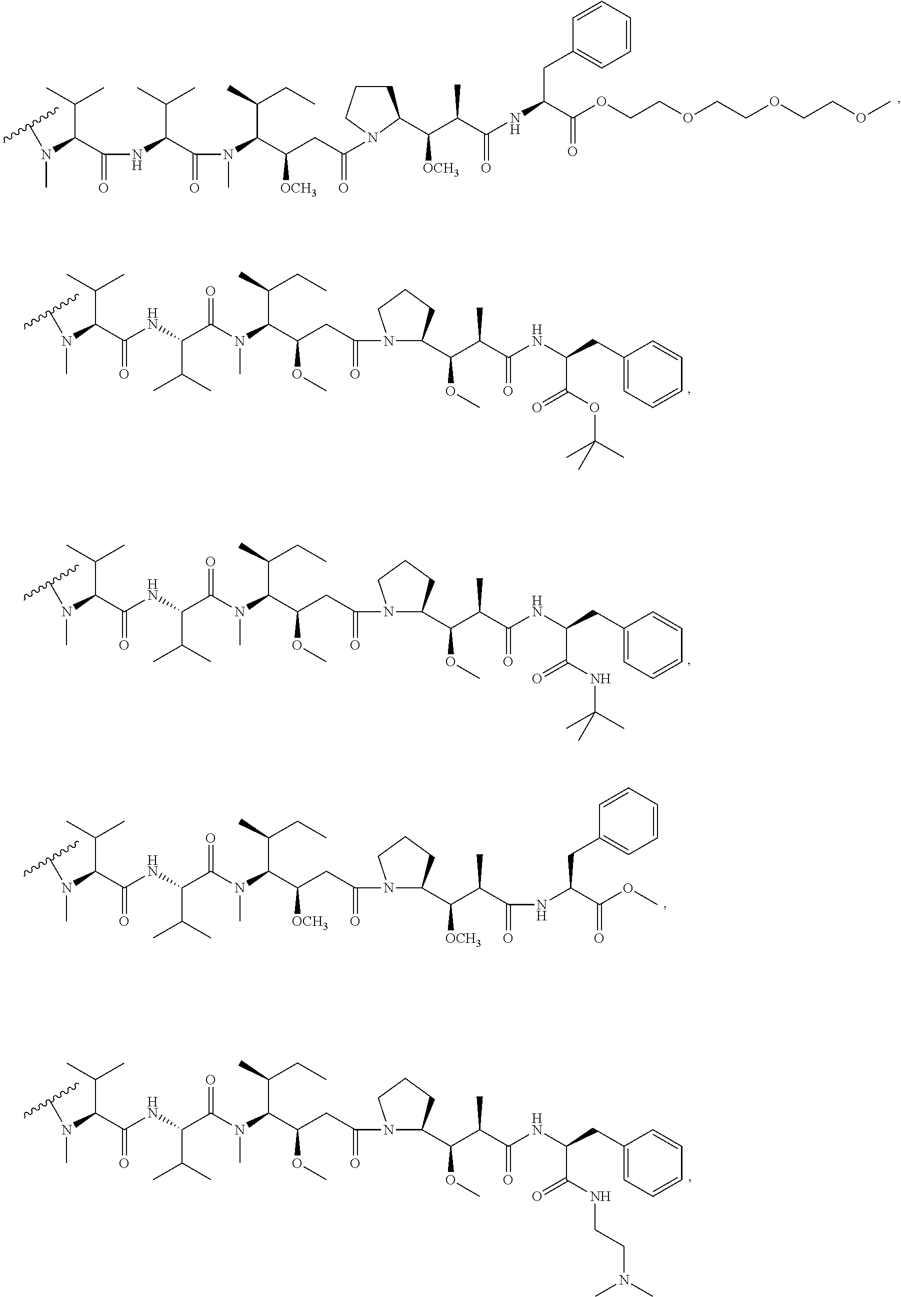

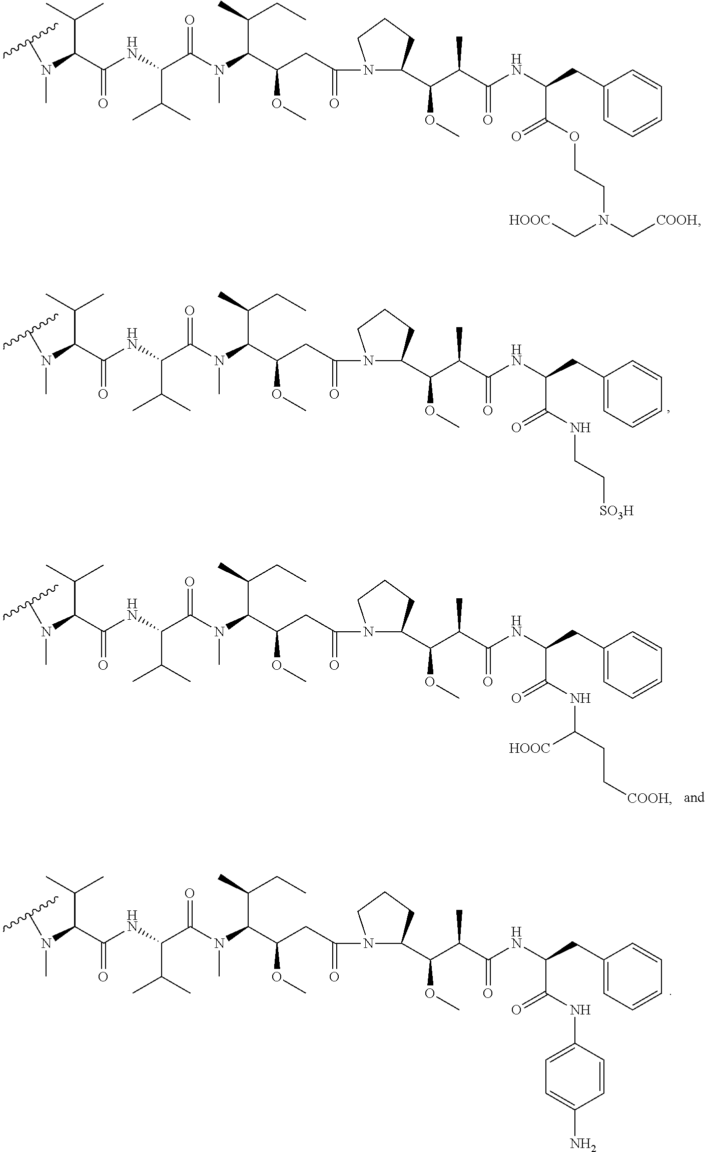

In one aspect, hydrophilic groups including but not limited to, triethylene glycol esters (TEG), as shown above, can be attached to the drug moiety at R¹¹. Without being bound by any particular theory, the hydrophilic groups assist in the internalization and non-agglomeration of the drug moiety.

Exemplary embodiments of ADCs of Formula I comprising an auristatin/dolastatin or derivative thereof are described in US 2005-0238649 A 1 and Doronina et al. (2006) Bioconjugate Chem. 17:114-124, which is expressly incorporated herein by reference. Exemplary embodiments of ADCs of Formula I comprising MMAE or MMAF and various linker components have the following structures and abbreviations (wherein “Ab” is an antibody, e.g., trastuzumab; p is 1 to about 8, “Val-Cit” is a valine-citrulline dipeptide; and “S” is a sulfur atom:

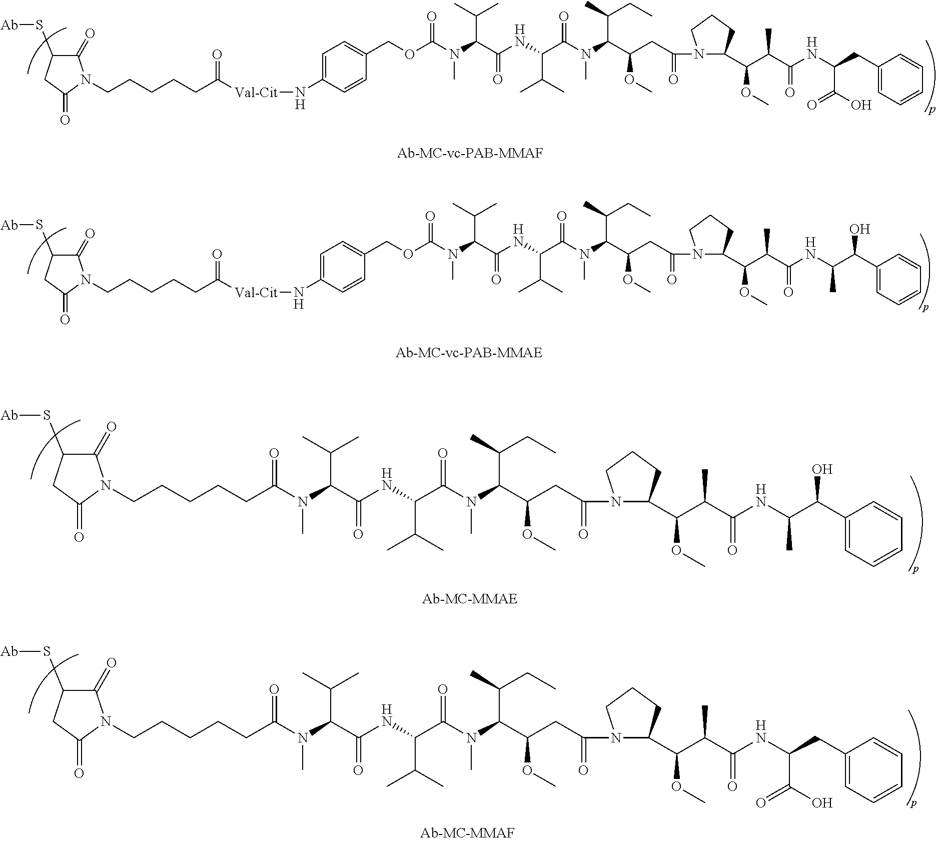

Exemplary embodiments of ADCs of Formula I comprising MMAF and various linker components further include Ab-MC-PAB-MMAF and Ab-PAB-MMAF. Interestingly, immunoconjugates comprising MMAF attached to an antibody by a linker that is not proteolytically cleavable have been shown to possess activity comparable to immunoconjugates comprising MMAF attached to an antibody by a proteolytically cleavable linker. See, Doronina et al. (2006) Bioconjugate Chem. 17:114-124. In such instances, drug release is believed to be effected by antibody degradation in the cell. Id.

Typically, peptide-based drug moieties can be prepared by forming a peptide bond between two or more amino acids and/or peptide fragments. Such peptide bonds can be prepared, for example, according to the liquid phase synthesis method (see E. Schroder and K. Lübke, “The Peptides”, volume 1, pp 76-136, 1965, Academic Press) that is well known in the field of peptide chemistry. Auristatin/dolastatin drug moieties may be prepared according to the methods of: US 2005-0238649 A1; U.S. Pat. No. 5,635,483; U.S. Pat. No. 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) J. Chem. Soc. Perkin Trans. 1 5:859-863; and Doronina (2003) Nat. Biotechnol. 21(7):778-784.

In particular, auristatin/dolastatin drug moieties of formula D_{F}, such as MMAF and derivatives thereof, may be prepared using methods described in US 2005-0238649 A1 and Doronina et al. (2006) Bioconjugate Chem. 17:114-124. Auristatin/dolastatin drug moieties of formula D_{E}, such as MMAE and derivatives thereof, may be prepared using methods described in Doronina et al. (2003) Nat. Biotech. 21:778-784. Drug-linker moieties MC-MMAF, MC-MMAE, MC-vc-PAB-MMAF, and MC-vc-PAB-MMAE may be conveniently synthesized by routine methods, e.g., as described in Doronina et al. (2003) Nat. Biotech. 21:778-784, and Patent Application Publication No. US 2005/0238649 A1, and then conjugated to an antibody of interest.

Examples of linkers reported in the scientific literature include methylene (CH₂)ₙ linkers (Hussey et al., J. Am. Chem. Soc., 2003, 125:3692-3693; Tamiz et al., J. Med. Chem., 2001, 44:1615-1622), oligo ethyleneoxy O(—CH₂CH₂O—)ₙ units used to link naltrexamine to other opioids, glycine oligomers of the formula —NH—(COCH₂NH)ₙCOCH₂CH₂CO—(NHCH₂CO)ₙNH— used to link opioid antagonists and agonists together ((a) Portoghese et al., Life Sci., 1982, 31:1283-1286. (b) Portoghese et al., J. Med. Chem., 1986, 29:1855-1861), hydrophilic diamines used to link opioid peptides together (Stepinski et al., Internat. J. of Peptide & Protein Res., 1991, 38:588-92), rigid double stranded DNA spacers (Paar et al., J. Immunol., 2002, 169:856-864) and the biodegradable linker poly(L-lactic acid) (Klok et al., Macromolecules, 2002, 35:746-759). The attachment of the tether to a compound can result in the compound achieving a favorable binding orientation. The linker itself may or may not be biodegradable. The linker may take the form of a prodrug and be tunable for optimal release kinetics of the linked drugs. The linker may be either conformationally flexible throughout its entire length or else a segment of the tether may be designed to be conformationally restricted (Portoghese et al., J. Med. Chem., 1986, 29:1650-1653).

Exemplary Linkers

A linker may comprise one or more linker components. Exemplary linker components include 6-maleimidocaproyl (“MC”), maleimidopropanoyl (“MP”), valine-citrulline (“val-cit” or “vc”), alanine-phenylalanine (“ala-phe”), p-aminobenzyloxycarbonyl (a “PAB”), N-Succinimidyl 4-(2-pyridylthio) pentanoate (“SPP”), N-succinimidyl 4-(N-maleimidomethyl)cyclohexane-1 carboxylate (“SMCC”), and N-Succinimidyl (4-iodo-acetyl) aminobenzoate (“SIAB”). Various linker components are known in the art, some of which are described below.

A linker may be a “cleavable linker,” facilitating release of a drug in the cell. For example, an acid-labile linker (e.g., hydrazone), protease-sensitive (e.g., peptidase-sensitive) linker, photolabile linker, dimethyl linker or disulfide-containing linker (Chari et al., Cancer Research 52:127-131 (1992); U.S. Pat. No. 5,208,020) may be used.

In some embodiments, a linker component may comprise a “stretcher unit” that links an antibody to another linker component or to a drug moiety. Exemplary stretcher units are shown below (wherein the wavy line indicates sites of covalent attachment to an antibody):

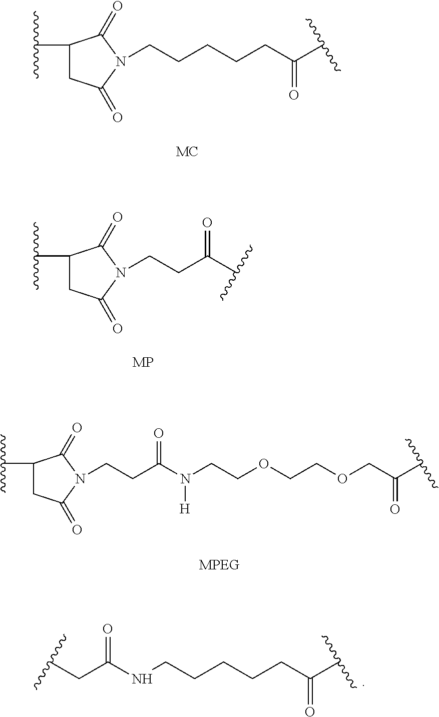 [SMILES: *C(=O)CCCCCN1C(=O)CC(*)C1=O ; *C(=O)CCN1C(=O)CC(*)C1=O ; *C(=O)COCCOCCNC(=O)CCN1C(=O)CC(*)C1=O ; *CC(=O)NCCCCCC(*)=O]

In some embodiments, a linker component may comprise an amino acid unit. In one such embodiment, the amino acid unit allows for cleavage of the linker by a protease, thereby facilitating release of the drug from the immunoconjugate upon exposure to intracellular proteases, such as lysosomal enzymes. See, e.g., Doronina et al. (2003) Nat. Biotechnol. 21:778-784. Exemplary amino acid units include, but are not limited to, a dipeptide, a tripeptide, a tetrapeptide, and a pentapeptide. Exemplary dipeptides include: valine-citrulline (vc or val-cit), alanine-phenylalanine (af or ala-phe); phenylalanine-lysine (fk or phe-lys); or N-methyl-valine-citrulline (Me-val-cit). Exemplary tripeptides include: glycine-valine-citrulline (gly-val-cit) and glycine-glycine-glycine (gly-gly-gly). An amino acid unit may comprise amino acid residues that occur naturally, as well as minor amino acids and non-naturally occurring amino acid analogs, such as citrulline. Amino acid units can be designed and optimized in their selectivity for enzymatic cleavage by a particular enzyme, for example, a tumor-associated protease, cathepsin B, C and D, or a plasmin protease.

In some embodiments, a linker component may comprise a “spacer” unit that links the antibody to a drug moiety, either directly or by way of a stretcher unit and/or an amino acid unit. A spacer unit may be “self-immolative” or a “non-self-immolative.” A “non-self-immolative” spacer unit is one in which part or all of the spacer unit remains bound to the drug moiety upon enzymatic (e.g., proteolytic) cleavage of the ADC. Examples of non-self-immolative spacer units include, but are not limited to, a glycine spacer unit and a glycine-glycine spacer unit. Other combinations of peptidic spacers susceptible to sequence-specific enzymatic cleavage are also contemplated. For example, enzymatic cleavage of an ADC containing a glycine-glycine spacer unit by a tumor-cell associated protease would result in release of a glycine-glycine-drug moiety from the remainder of the ADC. In one such embodiment, the glycine-glycine-drug moiety is then subjected to a separate hydrolysis step in the tumor cell, thus cleaving the glycine-glycine spacer unit from the drug moiety.

A “self-immolative” spacer unit allows for release of the drug moiety without a separate hydrolysis step. In certain embodiments, a spacer unit of a linker comprises a p-aminobenzyl unit. In one such embodiment, a p-aminobenzyl alcohol is attached to an amino acid unit via an amide bond, and a carbamate, methylcarbamate, or carbonate is made between the benzyl alcohol and a cytotoxic agent. See, e.g., Hamann et al. (2005) Expert Opin. Ther. Patents (2005) 15:1087-1103. In one embodiment, the spacer unit is p-aminobenzyloxycarbonyl (PAB). In certain embodiments, the phenylene portion of a p-amino benzyl unit is substituted with Qm, wherein Q is —C₁-C₈ alkyl, —O—(C₁-C₈ alkyl), -halogen, -nitro or -cyano; and m is an integer ranging from 0-4. Examples of self-immolative spacer units further include, but are not limited to, aromatic compounds that are electronically similar to p-aminobenzyl alcohol (see, e.g., US 2005/0256030 A1), such as 2-aminoimidazol-5-methanol derivatives (Hay et al. (1999) Bioorg. Med. Chem. Lett. 9:2237) and ortho- or para-aminobenzylacetals. Spacers can be used that undergo cyclization upon amide bond hydrolysis, such as substituted and unsubstituted 4-aminobutyric acid amides (Rodrigues et al., Chemistry Biology, 1995, 2, 223); appropriately substituted bicyclo[2.2.1] and bicyclo[2.2.2] ring systems (Storm, et al., J. Amer. Chem. Soc., 1972, 94, 5815); and 2-aminophenylpropionic acid amides (Amsberry, et al., J. Org. Chem., 1990, 55, 5867). Elimination of amine-containing drugs that are substituted at the a-position of glycine (Kingsbury, et al., J. Med. Chem., 1984, 27, 1447) are also examples of self-immolative spacers useful in ADCs.

In one embodiment, a spacer unit is a branched bis(hydroxymethyl)styrene (BHMS) unit as depicted below, which can be used to incorporate and release multiple drugs.

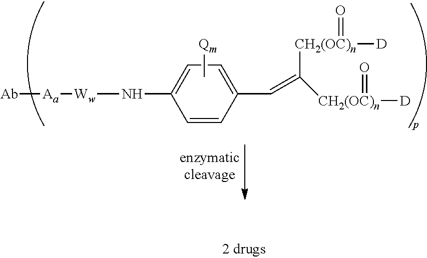

wherein Q is —C₁-C₈ alkyl, —O—(C₁-C₈ alkyl), -halogen, -nitro or -cyano; m is an integer ranging from 0-4; n is 0 or 1; and p ranges raging from 1 to about 20.

A linker may comprise any one or more of the above linker components. In certain embodiments, a linker is as shown in brackets in the following ADC Formula II

Ab([Aa-Ww-Yy]-D)ₚ II

wherein A is a stretcher unit, and a is an integer from 0 to 1; W is an amino acid unit, and w is an integer from 0 to 12; Y is a spacer unit, and y is 0, 1, or 2; and Ab, D, and p are defined as above for Formula I. Exemplary embodiments of such linkers are described in US 2005-0238649 A1, which is expressly incorporated herein by reference.

Exemplary linker components and combinations thereof are shown below in the context of ADCs of Formula II:

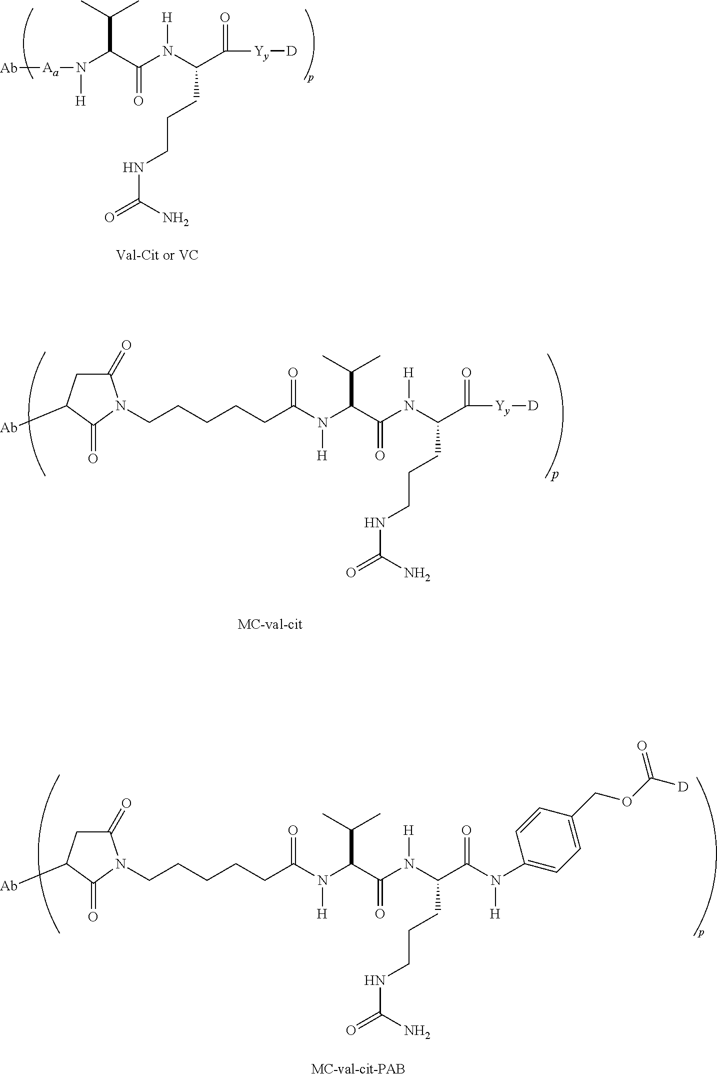

Linkers components, including stretcher, spacer, and amino acid units, may be synthesized by methods known in the art, such as those described in US 2005-0238649 A1.

The inventive method of treatment provides, in various embodiments, a therapeutic method that provides for horizontal modulation, as described above, wherein the second pro-apoptotic drug exerts cytotoxicity, inducing apoptosis, by a molecular mechanism other than the molecular mechanism of cytotoxicity exerted by the first pro-apoptotic anticancer drug moiety. Horizontal modulation is achieved when the first pro-apoptotic anticancer drug moiety and the second pro-apoptotic anticancer drug act on different biochemical cascades or processes that each separately can lead to apoptosis. For example, as shown in Table 2, below, the first pro-apoptotic anticancer drug moiety, such as a maytansinoid or an auristatin, can operate via an intrinsic apoptotic mechanism, such as microtubule depolymerization, and the second pro-apoptotic anticancer drug can be a drug that induces apoptosis via an extrinsic pathway, such as a Fas pathway, or a c-FLIP pathway, as are well-known in the art. Alternatively, if the first pro-apoptotic anticancer drug moiety exerts its effect via an extrinsic pathway, the second pro-apoptotic anticancer drug can be a drug that induces apoptosis via an intrinsic pathway, such as a caspase-independent pathway, or via a caspase-dependent pathway.

Accordingly, in various embodiments, the second pro-apoptotic drug is a drug that induces apoptosis via an extrinsic pathway; for example the second pro-apoptotic drug induces apoptosis via a Fas pathway, or the second pro-apoptotic drug induces apoptosis via a c-FLIP pathway. More specifically, the second pro-apoptotic drug can be CMH, E2, or δ-tocotrienol.

In other embodiments, the second pro-apoptotic drug is a drug that induces apoptosis via an intrinsic pathway; for example, the second pro-apoptotic drug can induce apoptosis via a caspase-independent pathway, or alternatively, the second pro-apoptotic drug can induce apoptosis via a caspase-dependent pathway. More specifically, the second pro-apoptotic drug can be E2, FTS, δ-tocotrienol, salinomycin, or curcumin.

Thus, in various embodiments, the second pro-apoptotic anticancer drug is FTS, CMH, E2, TMS, δ-tocotrienol, salinomycin, or curcumin. In various embodiments, the immunoconjugate is T-DM 1 and the second pro-apoptotic drug is FTS, CMH, E2, TMS, δ-tocotrienol, or curcumin. When the immunoconjugate is T-DM1, the second pro-apoptotic drug can be E2, FTS, δ-tocotrienol, or TMS; or, more specifically, the immunoconjugate is T-DM1 and the second pro-apoptotic drug is FTS.

The present invention provides methods wherein administering the immunoconjugate and the second pro-apoptotic drug can have a synergistic effect. In some embodiments, the synergy is achieved by the use of horizontal modulation.

However, even when the two pro-apoptotic anticancer agents act with vertical modulation, synergistic or additive effects can be achieved. Furthermore, for horizontal modulation to be achieved, it may still be possible for the two anticancer agents to both operate via an extrinsic or an intrinsic pathway, provided that they do not both operate on the same process within the pathway, wherein adaptive mutation to provide drug resistance would still need to occur via two simultaneous mutations to confer resistance.

In various embodiments, the invention provides a method of treatment of cancer, comprising administration to a cancer patient of trastuzumab-DM 1 (T-DM1), an embodiment of the above-described covalent conjugate of a targeting monoclonal antibody, trastuzumab (Herceptin®) bonded via a linker moiety to a first pro-apoptotic anticancer drug moiety, a maytansinoid ansa macrolide. This conjugate, T-DM1, is disclosed by the inventors herein to provide, in a combination therapy regimen using as a second pro-apoptotic anticancer drug farnesyl-thiosalicylate FTS is found to provide a surprisingly high synergistic effect. In yet another aspect, a combination therapy of E2 and T-DM 1 provides a surprisingly high synergistic effect. In yet another aspect, a combination therapy of CMH and T-DM1 provides a surprisingly high synergistic effect.

In the above example of T-DM1, the first pro-apoptotic anticancer drug moiety, a close analog of maytansine, is coupled via a linker moiety to trastumuzab (Herceptin). The linker does not incorporate a disulfide bond, thus is considered to be a non-reducible linker moiety according to the meaning herein; i.e., that the easily reducible disulfide bond is not present. Accordingly, in various embodiments, the invention provides a covalent conjugate consisting essentially of a monoclonal antibody moiety covalently coupled via a non-reducible linker with the first pro-apoptotic anticancer drug moiety, wherein non-reducible refers to the absence of a disulfide bond or other group wherein reduction under biological conditions is considered likely to occur.

In Table 1, below, the results of various combinations showing additive and synergistic effects are shown

TABLE 1

| | | | | Dose Effect | | | | | | |
|---|---|---|---|---|---|---|---|---|---|---|
| | Apoptotic | | | Parameters | | Combination Index Values | | | | |
| Cell Line | Agent | Ratio | Dm | m | r | ED50 | ED75 | ED 90 | ED95 | Interaction |
| MCF-7 | TMS + FTS | TMS:FTS (1 μM:1 μM) | 4.806 | 1.74598 ± 0.07883 | 0.9939 | 0.988 | 0.939 | 0.898 | 0.865 | Additive |
| | TMS + CMH | TMS:CMH (1 μM:10 μM) | 5.839 | 1.17019 ± 0.07844 | 0.9933 | 0.321 | 0.343 | 0.368 | 0.386 | Synergy |
| | FTS + CMH | FTS:CMH (1 μM:1 μM) | 17.402 | 1.39405 ± 0.10368 | 0.9891 | 0.351 | 0.399 | 0.454 | 0.495 | Synergy |
| T47D | TMS + FTS | TMS:FTS (1 μM:1 μM) | 16.046 | 1.552969 ± 0.13846 | 0.9888 | 2.408 | 1.839 | 1.443 | 1.246 | Antagonistic |
| | TMS + CMH | TMS:CMH (1 μM:10 μM) | 4.885 | 1.02624 ± 0.15858 | 0.9769 | 0.242 | 0.328 | 0.452 | 0.566 | Synergy |
| | FTS + CMH | FTS:CMH (1 μM:1 μM) | 16.447 | 1.3291 ± 0.11190 | 0.9895 | 0.730 | 0.658 | 0.602 | 0.571 | Mild Synergy |
| LTED | TMS + FTS | TMS:FTS (1 μM:1 μM) | 5.891 | 0.76939 ± 0.01125 | 0.9995 | 0.911 | 0.889 | 0.869 | 0.858 | Mild Synergy |
| | TMS + CMH | TMS:CMH (1 μM:10 μM) | 0.088 | 0.55289 ± 0.04221 | 0.9942 | 0.391 | 0.223 | 0.177 | 0.206 | Synergy |
| | TMS + E2 | TMS:E2 (1 μM:10 μM) | 1.025 | .74308 ± 0.0681 | 0.9836 | 0.322 | 0.566 | 1.009 | 1.498 | Additive |
| | TMS + T-DM1 | TMS:T-DM1 (1 μM:1 ng) | 7.337 | 0.134089 ± 0.16995 | 0.9843 | 1.034 | 1.024 | 1.015 | 1.010 | Additive |
| | FTS + CMH | FTS:CMH (1 μM:1 μM) | 20.964 | 1.20938 ± 0.10418 | 0.9855 | 0.811 | 0.763 | 0.719 | 0.692 | Mild Synergy |
| | FTS + E2 | FTS:E2 (10 μM:1 μM) | 14.082 | 0.78722 ± 0.12684 | 0.9408 | 0.784 | 0.609 | 0.491 | 0.429 | Synergy |
| | FTS + T-DM1 | FTS:T-DM1 (1 μM:10 ng) | 63.540 | 1.01308 ± 0.05397 | 0.9944 | 0.400 | 0.266 | 0.178 | 0.135 | Strong Synergy |
| | E2 + CMH | E2:CMH (1 μM:10 μM) | 21.759 | 1.27183 ± 0.05093 | 0.9976 | 1.002 | 0.753 | 0.684 | 0.663 | Additive |
| | E2 + T-DM1 | E2:T-DM1 (1 μM:10 ng) | 1.349 | 0.64658 ± 0.00913 | 0.9996 | 0.421 | 0.329 | 0.299 | 0.295 | Synergy |
| | CMH + T-DM1 | CMH:T-DM1 (1 μM:10 ng) | 33.937 | 1.22358 ± 0.02137 | 0.9995 | 0.144 | 0.123 | 0.115 | 0.113 | Strong Synergy |

Strong synergy is observed in combinations of T-DM1 with FTS and CMH. The methods used to determine the degree of synergy, and the results of various studies, are described below and are displayed in graphic form in the Figures.

In other embodiments, a first pro-apoptotic drug moiety can be covalently coupled via a reducible linker with the first pro-apoptotic drug moiety. By a “reducible” linkage is meant that it is believed or expected that such a linker is likely to be cleaved by a reductive process possible to occur under biological conditions, i.e., reduction of a disulfide bond. In such conjugates, it is contemplated that the targeting moiety, e.g., a monoclonal antibody, conveys the first pro-apoptoic drug moiety to the desired target, e.g., the HER2 or related receptor in the case of hormone-resistant breast cancer, whereupon cleavage of the drug from the targeting moiety can occur, freeing the drug such that it can more readily diffuse through tissue, pass cell membranes, and the like.

In various embodiments, the method of treatment of a cancer can be used when

the cancer is a breast cancer. By a breast cancer is meant any of the numerous types of cancers that can afflict mammary tissue. In other embodiments, other types of cancers can be treated similarly, i.e., by use of a targeting moiety specific for an epitope characteristic of that type of cancer, such as an overexpressed receptor, wherein the monoclonal antibody or other targeting moiety chosen is covalently coupled to a first pro-apoptotic drug, the resulting conjugate being administered in conjunction with a second pro-apoptotic drug. In these embodiments as well, a horizontal modulation approach is believed to provide for a lower probability of development of resistance by the targeted cancer cells.

### Pro-Apoptotic Mechanisms in Cancer Cells

Specific pro-apoptotic drugs that can be used in a therapeutic method of the invention are described in greater detail below.

### FTS

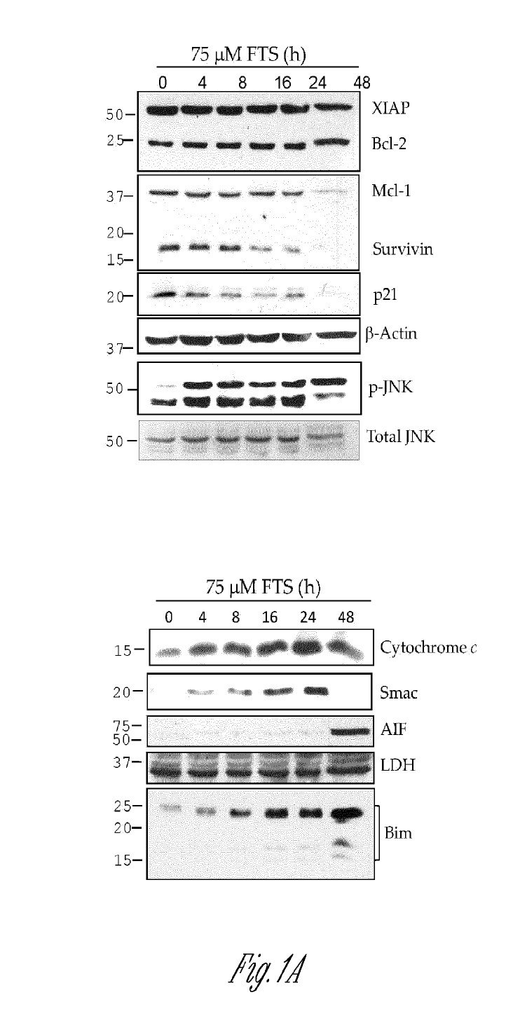

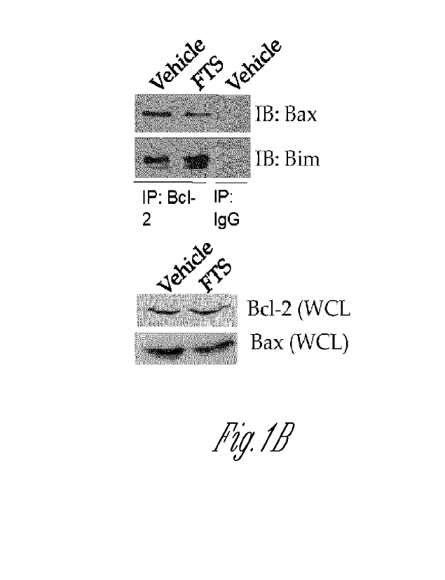

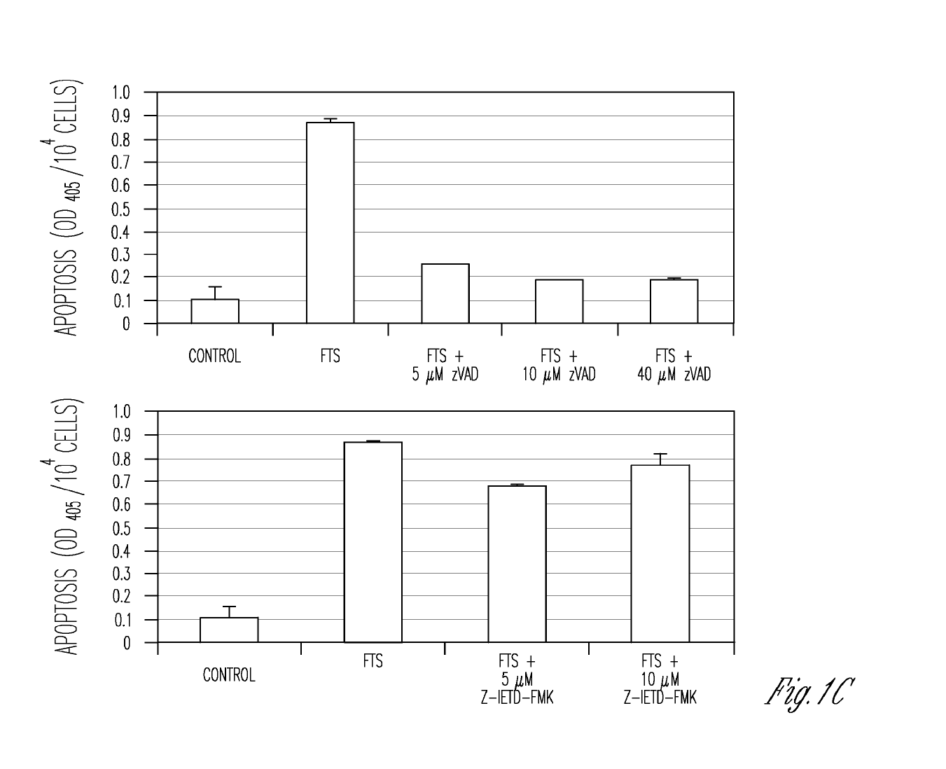

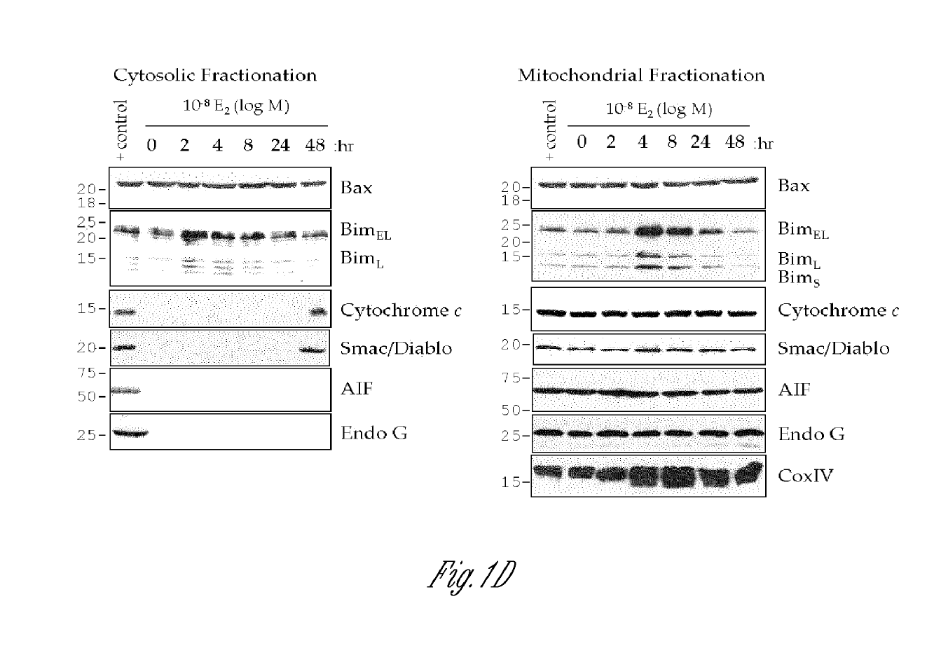

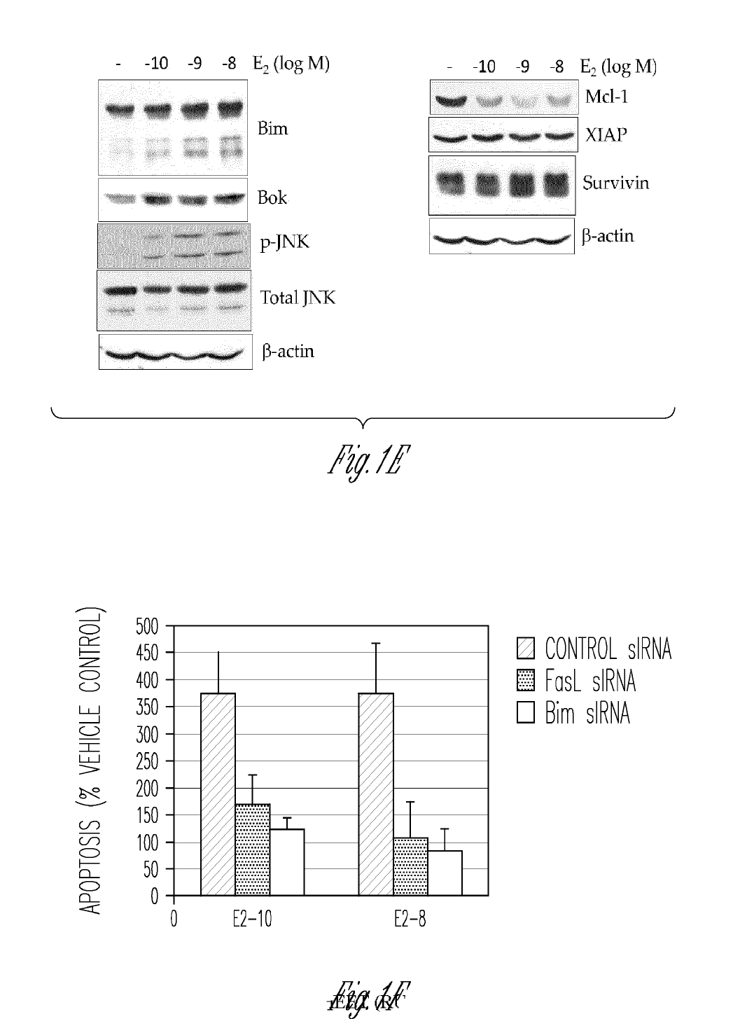

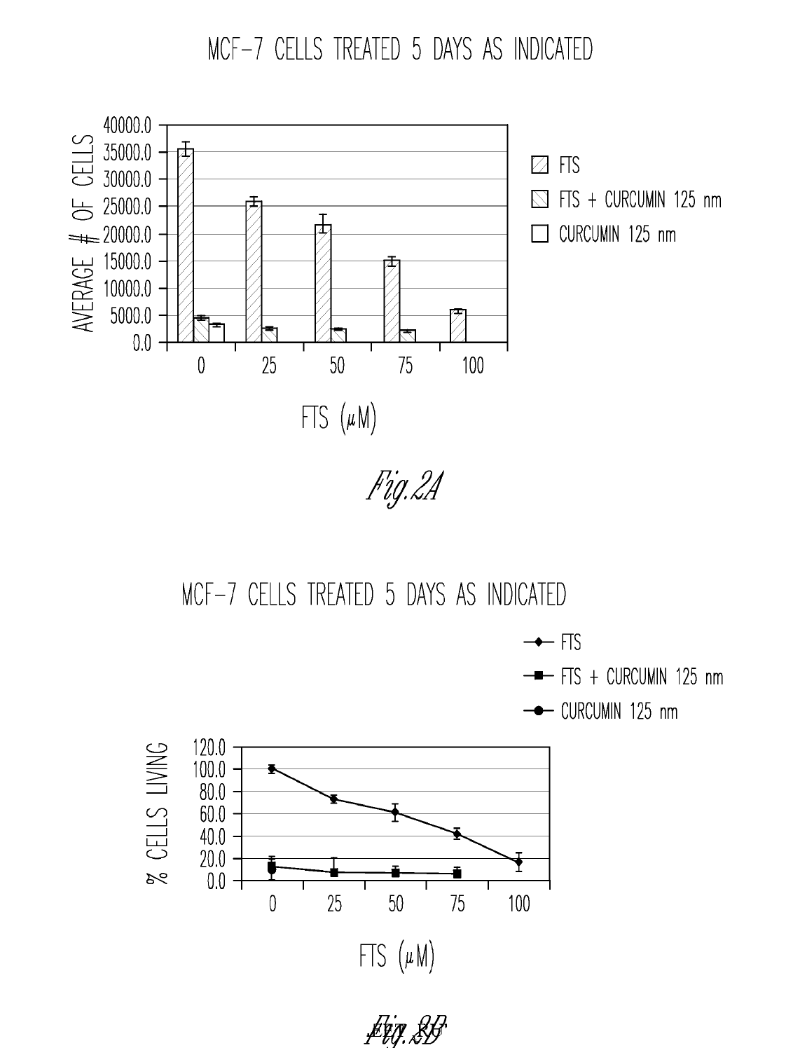

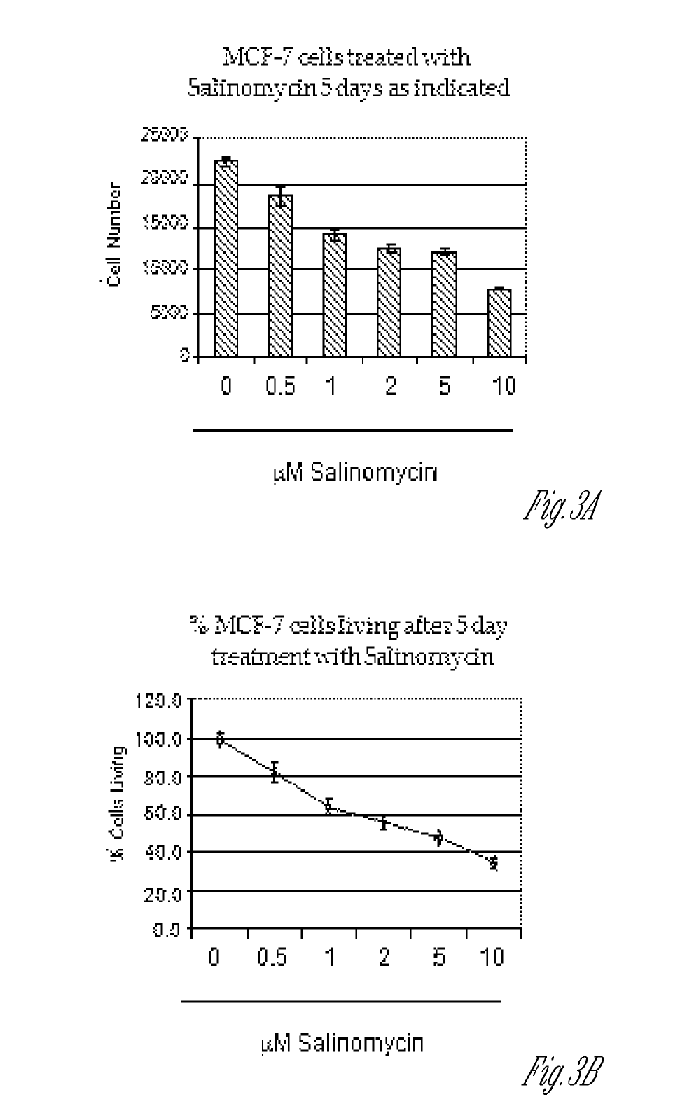

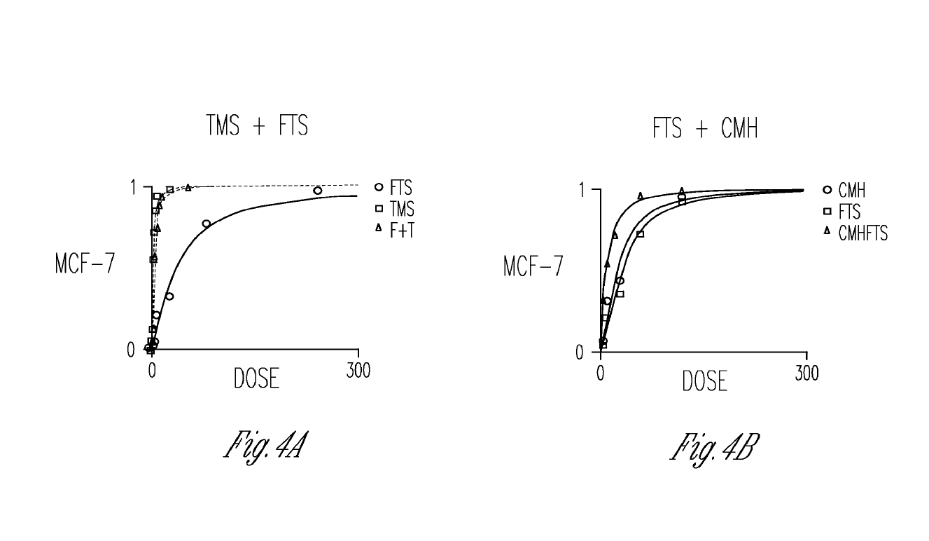

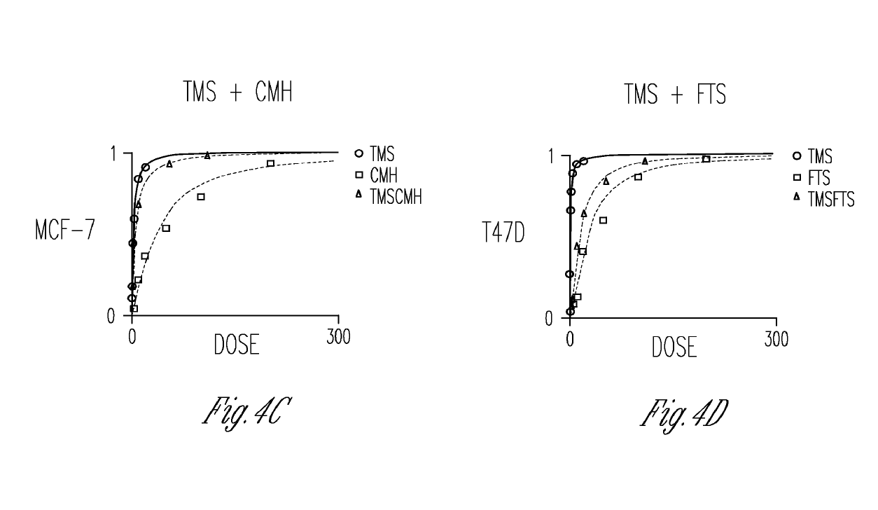

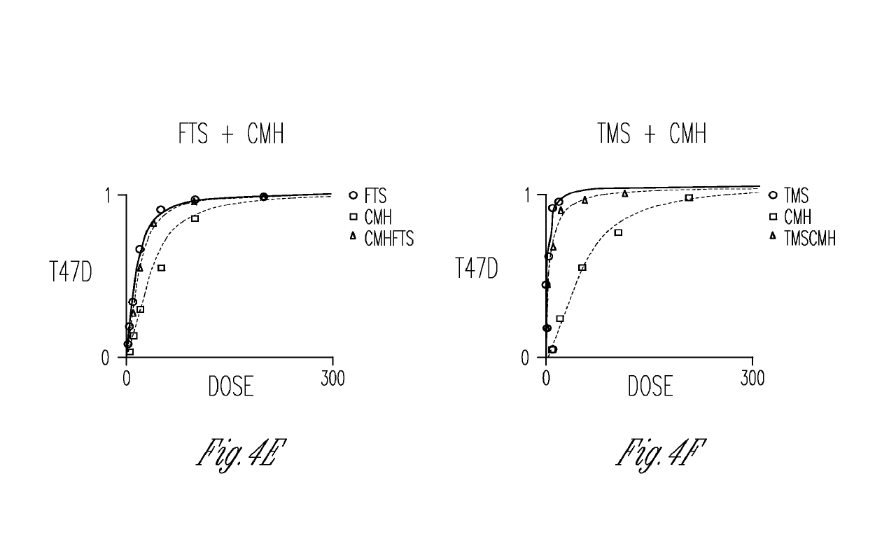

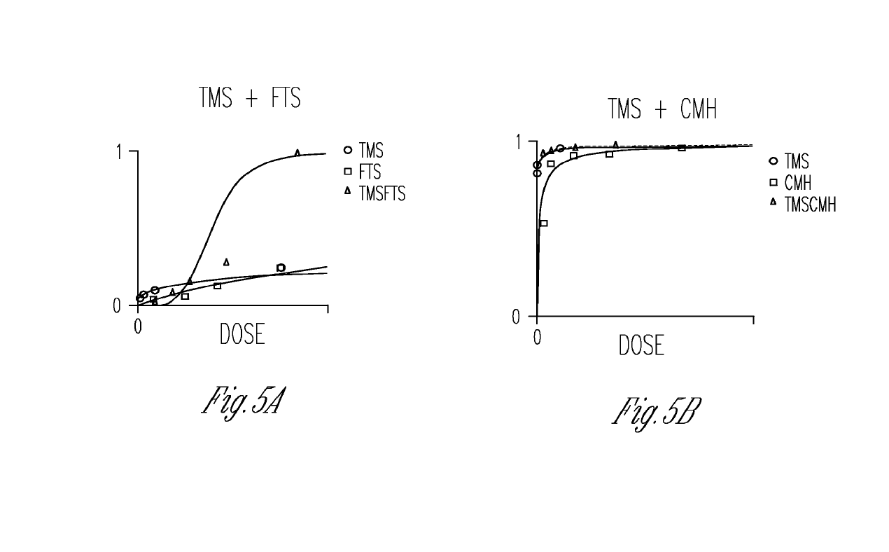

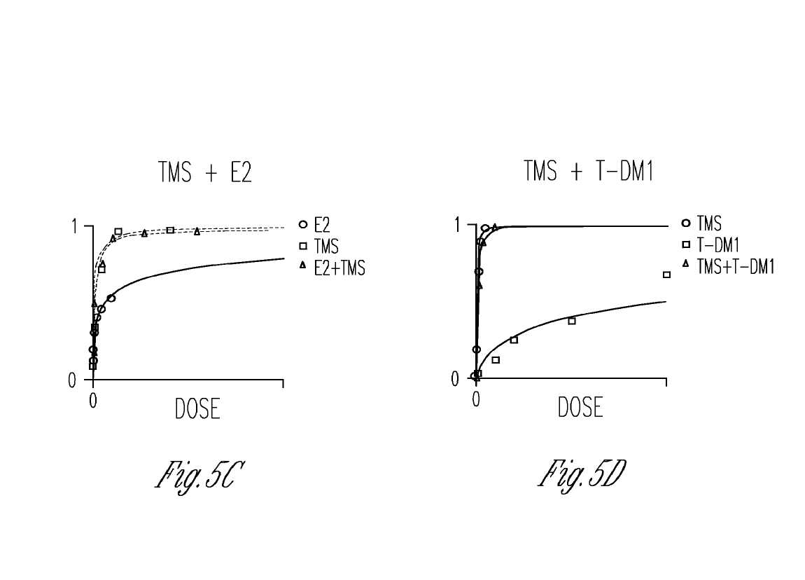

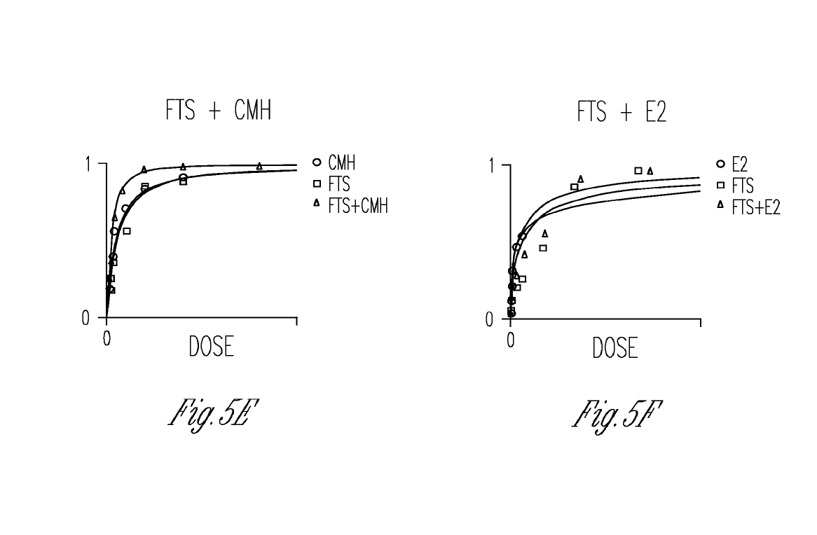

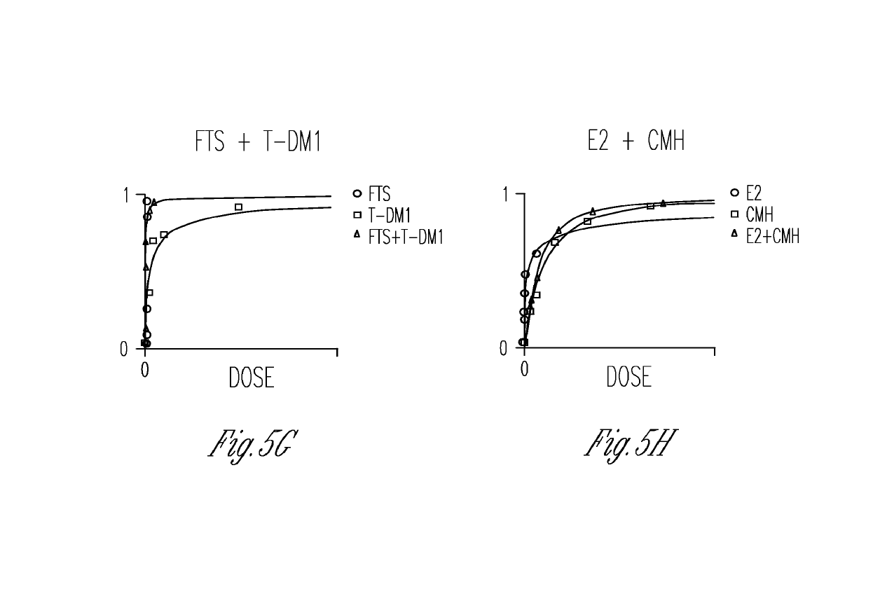

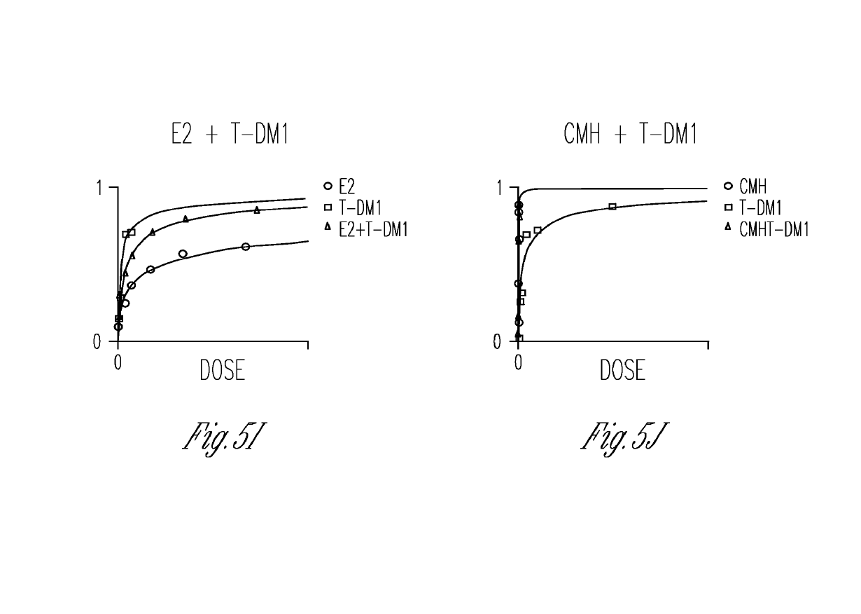

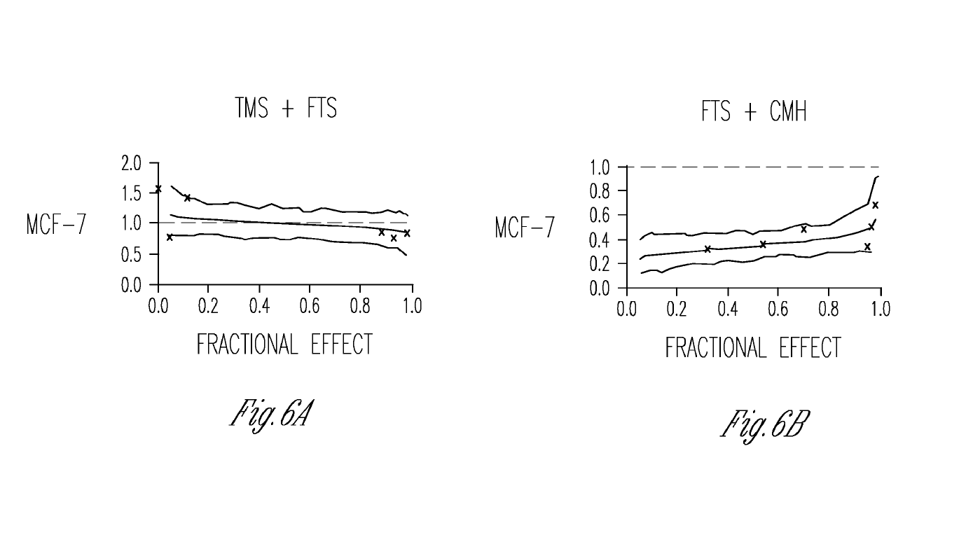

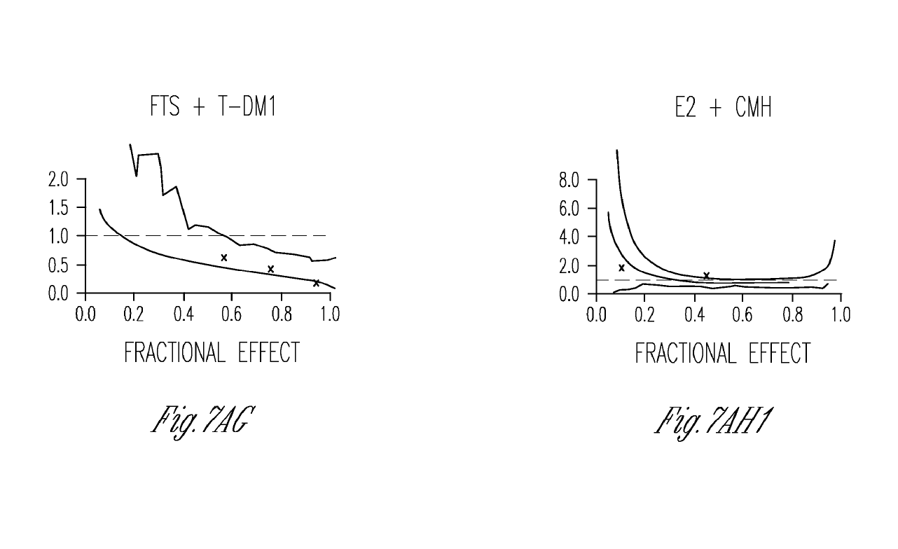

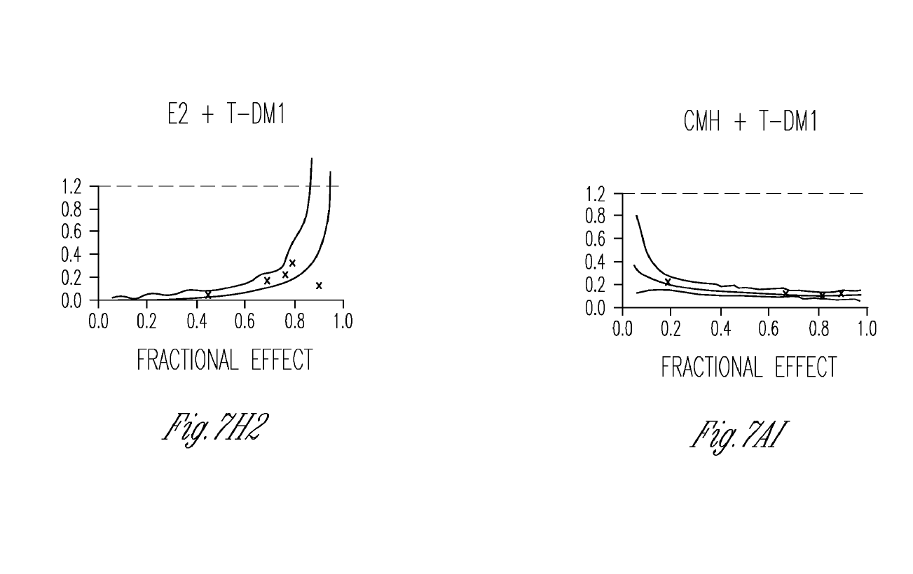

The inventors herein initially examined the class of proteins which influences MOMP (mitochondrial outer membrane pore) formation, a key component of the intrinsic pathway of apoptosis. Pro-apoptotic members such as Bax, Bim, and Bak, promote the release of Cytochrome c from the mitochondria, whereas anti-apoptotic members, such as Bcl-2 and Mcl-1, prevent release. The balance of pro-apoptotic and anti-apoptotic Bcl-2 proteins therefore influences the fate of the cell. LTED cells were treated with 75 μM FTS for 0, 4, 8, 16, 24 and 48 h with examination of cytosolic fractions (FIG. 1A). Apoptotic signaling resulted in a decrease in Mcl-1 by 24 h, and an increase in Bim, but Bcl-2 was unchanged. Phospho JNK increased over 48 hours and p21 showed a steady decrease starting at 4 h and lasting to the 48 h time point (FIG. 4A). Survivin decreased starting at 8 to 16 h and reached undetectable levels at 24 and 48 hours, but XIAP did not change (FIG. 1A).

To ascertain whether Bax was activated in FTS-treated LTED cells, Bcl-2 was immunoprecipitated and then probed for Bim and Bax (See FIG. 1B). Probed cell extracts were probed with the use of an antibody that recognizes only the conformationally altered Bax protein. As shown in FIG. 1B, Bax underwent a conformational change in FTS-treated cells, which would facilitate MOMP. The pro-apoptotic effect of Bim is predominantly through its binding to Bcl-2 that ablates Bcl-2 pro-survival function [16,17]. As shown in FIG. 1B we found the interaction between Bim and Bcl-2 is increased, whereas the interaction between Bax and Bcl-2 was reduced. As evidence of the exodus of proteins through mitochondrial membrane pores, Cytochrome c and Smac levels in the cytosol were increased at 24 and 48 h, times when Mcl-1 was decreased and Bim, increased (FIG. 1A). Apoptosis inducing factor (AIF), another important cell death component, appeared in the cytosol only at 48 h. After showing these effects on MOMP, other key factors involved in apoptosis were examined.

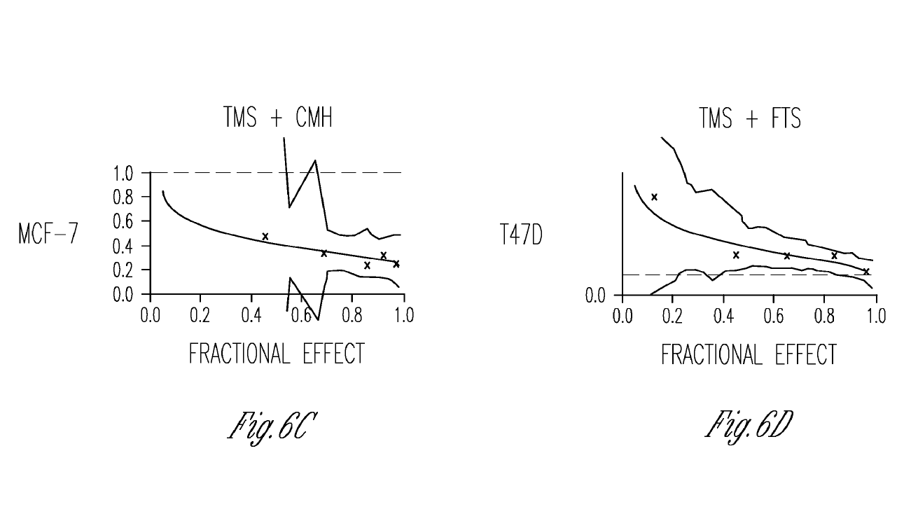

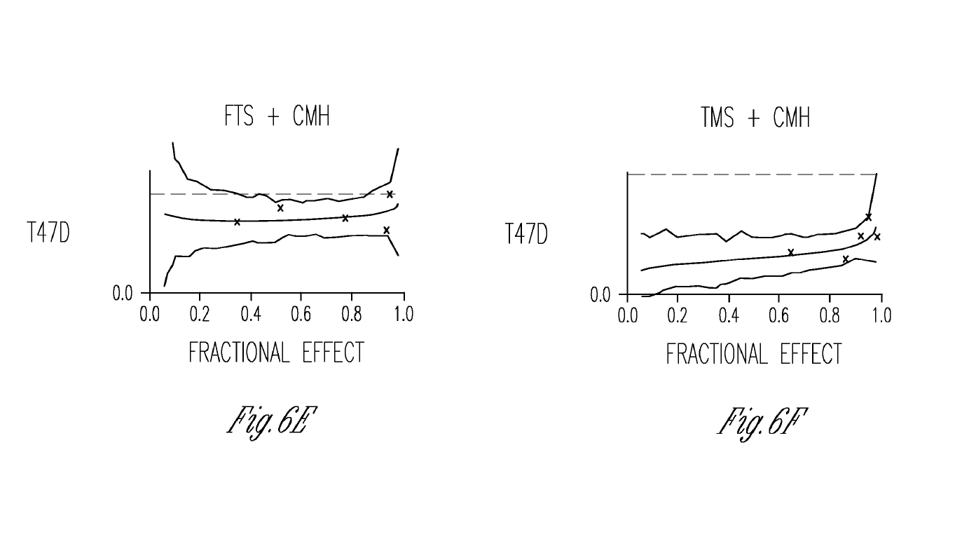

FTS has been reported to invoke cell death through caspase activation in non-breast tissues [18-20]. To address whether FTS was promoting death of breast cancer cells by activation of caspases LTED cells were treated with either vehicle, FTS, or FTS in the presence of increasing concentrations of the pan-caspase inhibitor z-VAD-fmk (See FIG. 1C top panel). It was found that z-VAD-fmk blocked FTS-induced apoptosis. Prior reports had suggested that in other cancers, FTS induces apoptosis through the death receptor, as evidenced by increases in caspase-8 [18-20]. In breast cancer cells, the death receptor pathway did not appear involved since no substantial caspase-8 changes occurred (See FIG. 1C lower panel). Caspase-8 activity does appear to increase; however this is not inhibited by Z-IETD-FMK. See FIG. 2, showing a time course bar graph (2A) and a cell viability versus concentration curve (2B) displaying (FIG. 2A) the effect of FTS and curcumin in combination on wild type MCF-7 cells; and (FIG. 2B) the effect of FTS alone or in combination with curcumin on MCF-7 cell viability.

### Estradiol

Which pro-apoptotic factors were critical for estradiol-induced apoptosis in LTED cells were examined. Cells were treated with estradiol for 0, 2, 4, 8, 24 and 48 h and cytosolic fractions prepared. Bim_{EL} and Bim_{L} increased at early time points (FIG. 1D, compare 4, 8 h to control) but Bax did not. Mitochondrial fractions (FIG. 1D, right panel), confirmed the increase in Bim isoforms. By the 48 h time point, Cytochrome c and Smac/Diablo were released into the cytosol, demonstrating that a component of estradiol apoptosis is mediated via the mitochondrial pathway. Because Bim appeared to be critical for estradiol-mediated apoptosis, we carried out a titration of E2, probed for Bim and found it increased over time (See FIG. 1E). Bim knockdown also blocked apoptosis (FIG. 1F). Upstream modulators of apoptosis were examined, and it was found that the phosphorylated form of JNK was increased (See FIG. 1E). Bok, a pro-apoptotic protein was also increased in a concentration-dependent manner (See FIG. 1E). It was also found that the anti-apoptotic factor Mcl-1 was decreased by the addition of estrogen, but not XIAP or survivin.

Previously published data indirectly implicate the extrinsic death receptor pathway in estradiol induced apoptosis. These prior data demonstrated that estradiol increased the levels of FAS-ligand in LTED cells, that FAS was present, and that the pathway could be activated by a monoclonal antibody against FAS which stimulated apoptosis. Herein, direct evidence is provided of FAS/FAS-ligand involvement by demonstrating that an siRNA against Fas-ligand partially abrogates estradiol induced apoptosis (FIG. 1F). Accordingly, estradiol initiates apoptosis by both extrinsic and intrinsic pathway activation.

Based on past and current results and a literature review, the actions of each of the agents on mitochondrial mediated apoptosis and the actions of the extrinsic, death receptor mediated apoptotic pathway are summarized in Table 2, below.

### Salinomycin

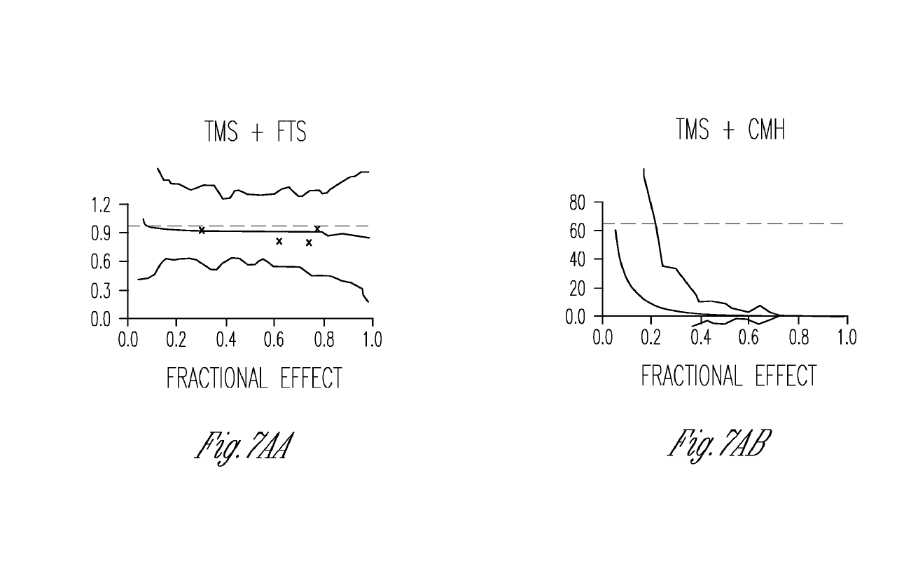

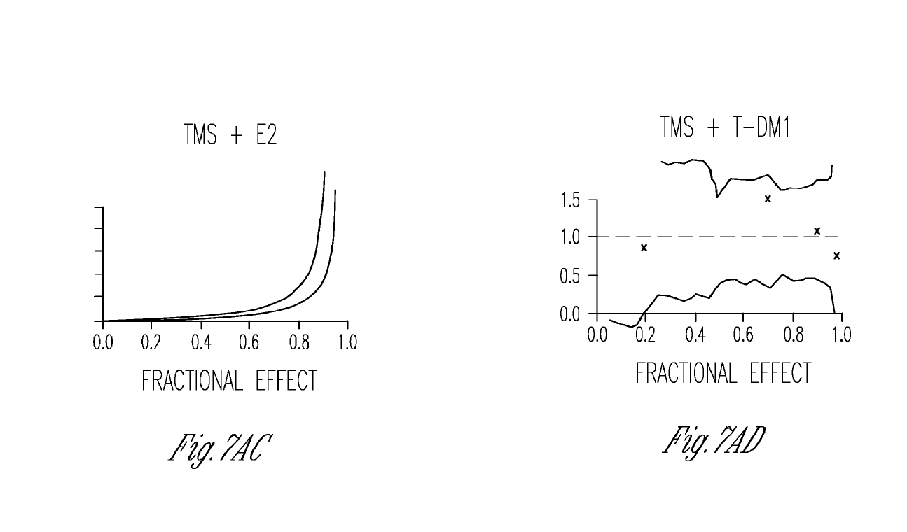

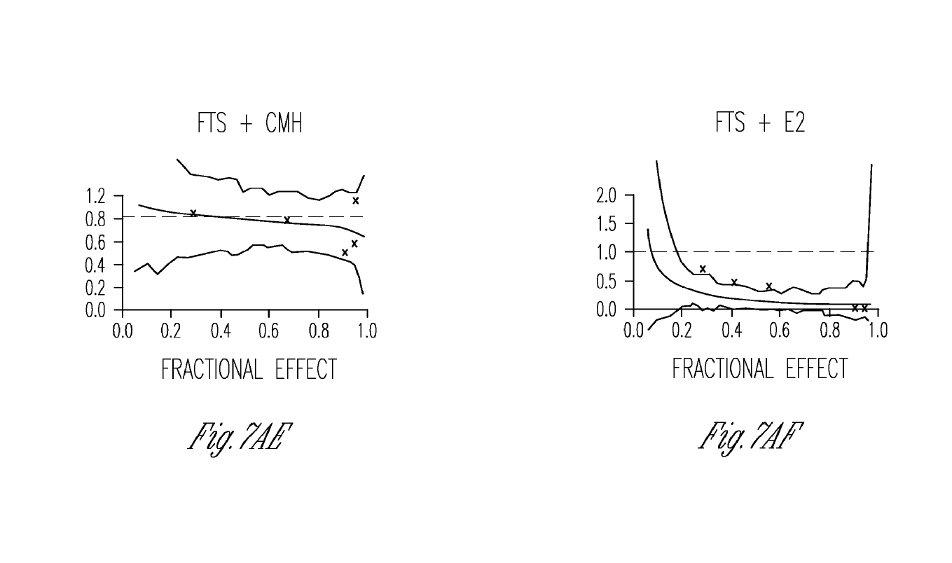

Salinomycin acts in different biological membranes, including cytoplasmic and mitochondrial membranes, as a ionophore with strict selectivity for alkali ions and a strong preference for potassium, thereby promoting mitochondrial and cellular potassium efflux and inhibiting mitochondrial oxidative phosphorylation. A recent study revealed that salinomycin induces apoptosis and overcomes apoptosis resistance in human cancer cells of different origin. First, it was demonstrated that salinomycin at doses lower than used by Gupta et al. induces massive apoptosis in CD4+ T-cell leukemia cells isolated from patients with acute T-cell leukemia. See http://www.scitopics.com/New_mission_for_salinomycin_in_cancer.html. It is believed that salinomycin act by an intrinsic, caspase independent pathway to induce apoptosis. Salinomycin activates a distinct and unconventional pathway of apoptosis in cancer cells that is not accompanied by cell cycle arrest, and that is independent of tumor suppressor protein p53, caspase activation, the CD95/DC95 ligand system and the 26S proteasome. This might be one reason why salinomycin can overcome multiple mechanisms of drug and apoptosis resistance in human cancer cells. Many cancer cells harbor or acquire multiple mechanisms of apoptosis resistance mediated by loss of p53 and overexpression of Bcl-2, P-glycoprotein or 26S proteasomes with enhanced proteolytic activity. Salinomycin, however, seems to be able to overcome these mechanisms of drug and apoptosis resistance. See FIG. 3, showing a time course bar graph (3A) and a cell viability versus concentration curve (3B) displaying the effect of salinomycin on MCF-7 cells.

TABLE 2
Molecular Mechanisms of Action of Selected Pro-Apoptotic Drugs

| Apoptotic | Intrinsic death | Extrinsic death |
|---|---|---|
| Agent | pathway | pathway |
| CMH/Droxinostat | No | Yes |
| | | blocks c-FLIP |
| E₂ | Yes | Yes |
| | Caspase-dependent | Fas pathway |
| FTS (Salirasib) | Yes | No |
| | Caspase-dependent | |
| T-DM1 | Yes | No |
| | Caspase-dependent | |
| δ-Tocotrienol | Yes | Yes |
| | Caspase-dependent | Fas pathway |
| | Caspase-independent | |
| TMS | Yes | No |
| | Caspase-independent | |

In various embodiments, the invention provides a method of treatment as described above, wherein the second pro-apoptotic anticancer drug is FTS, CMH, E2, TMS, δ-tocotrienol, curcumin, or salinomycin. Structures of these compounds, the rationale for the selection, of which is described above, are provided below. It is believed that certain of these drugs, such as salinomycin and curcumin, can act on stem cells.

### Curcumin

Curcumin, the active ingredient from the spice turmeric (Curcuma longa Linn), is a potent antioxidant and anti-inflammatory agent. It has been recently demonstrated to possess discrete chemopreventive activities. However, the molecular mechanisms underlying such anticancer properties of curcumin still remain unrealized, although it has been postulated that induction of apoptosis in cancer cells might be a probable explanation. In the current study, curcumin was found to decrease the Ehrlich's ascites carcinoma (EAC) cell number by the induction of apoptosis in the tumor cells as evident from flow-cytometric analysis of cell cycle phase distribution of nuclear DNA and oligonucleosomal fragmentation. Probing further into the molecular signals leading to apoptosis of EAC cells, we observed that curcumin is causing tumor cell death by the up-regulation of the proto-oncoprotein Bax, release of cytochrome c from the mitochondria, and activation of caspase-3. The status of Bcl-2 remains unchanged in EAC, which would signify that curcumin is bypassing the Bcl-2 checkpoint and overriding its protective effect on apoptosis. Thus, it is believed that curcumin can induce apoptosis via an intrinsic, caspase-dependent pathway.

See http://www.ncbi.nlm.nih.gov/pubmed/11676493.

### Rationale for the Choice of Combination Partners

Agents were chosen that would invoke multiple forms of cell death, such that horizontal modulation could be achieved with combination therapeutic methods of the invention. FTS (Salirasib) invokes caspase-dependent death in cancer cells through the mitochondrial cell death pathway[11,12]. FTS promotes apoptosis in MCF-7 cells and tumor xenografts[13]. CMH is a small molecule inhibitor of Cellular FLICE (FADD-like IL-1beta-converting enzyme)-inhibitory protein (c-FLIP) and CMH can activate caspase-8 and -10 by inhibiting c-FLIP [14,15]. Part of the mechanism of CMH's ability to sensitize cells to death ligands is through its ability to inhibit HDAC3, HDAC6 and HDAC8 [15]. TMS is an agent that invokes a predominantly caspase-independent death through the mitochondrial death pathway via microtubule inhibition [16,17]. TMS is effective for reducing the growth of TamR resistant breast cancer tumor xenografts[17]. Estradiol was shown to induce apoptosis of long term estrogen deprived cells through the mitochondrial cell death pathway [18,19] and also the Fas death receptor pathway[19]. Prior work had demonstrated that estradiol promotes apoptosis of long-term estrogen deprived cells in vitro [18-22], in xenograft models[23,24] as well as patients [25]. It has been shown the maytansinoid-antibody conjugates inhibit cell proliferation by arresting breast cancer cells in mitotic prometaphase/metaphase through microtubule depolymerization[26].

When cells are arrested in the cell cycle for a prolonged period this can lead to apoptosis[27,28]. T-DM1, a drug antibody conjugate of Trastuzumab-DM1 (a maytansine derivative), was shown effective for reducing HER2 expressing xenografts[29] as well as effective in patients with HER2 advanced breast cancer[30]. Breast cancer cell lines that have been deprived of estrogen in vivo in xenograft models by the administration of the aromatase inhibitor letrozole have led to upregulation of HER2 signaling[31,32]. Additionally, HER2 was shown to be upregulated in breast cancer patients during treatment with aromatase inhibitors[33]. Thus, breast cancer cells that have undergone estrogen deprivation long-term, have increased levels of HER2 making them sensitive to T-DM 1.

### Synergy Analysis

The analysis of synergistic effects in the combination therapy has focused on three cell lines, MCF-7, T47D, and LTED. The MCF-7 and T47D cell lines represent models of non-adapted breast cancer. The LTED (Long-term estrogen-deprived) cell line represent endocrine resistance after long-term estrogen deprivation. The following drug agents were used in the non-adapted cell lines: Farnesylthiosalicylic acid (FTS, Salirasib), 4-(4-Chloro-2-methylphenoxy)-N-hydroxybutanamide (CMH), and 2, 4, 3′,5′-tetramethoxystilbene (TMS). For the adapted cell lines, Estradiol (E2) and Trastuzumab-DM1 (T-DM1) were also included.

With the emergence of the stem cell population as an important component of tumor growth, the effects of curcumin were also examined in the in vitro system. Curcumin induces a dose and time dependent inhibition of colony formation and stem cell sphere formation in non-breast cancer studies. For this reason, we began preliminary studies of the effect of this agent in breast cancer long term estrogen deprived (LTED) cells. We examined the effects of curcumin in a dose response fashion. This agent was highly potent in reducing cell number with an initial effects observed at 250 nM. Later studies showed effects at 125 nM. We then examined curcumin in combination with FTS. The doses of FTS included vehicle, 25, 50, 75, and 100 μM. FTS alone reduced cell number to 14% of control. Curcumin alone at 125 nM reduced cell number to a similar extent. Because of the high degree of efficacy of curcumin, it was not possible to determine if FTS caused either additive effects or synergy in these experiments.

Salinomycin is another agent shown to be effective in killing stem cells. This agent was less potent than curcumin and exhibited a 50% inhibitory effect at 2 μM on MCF-7 cells. See FIG. 3. MCF-7-5C cells which had previously been shown to undergo apoptosis in vivo were used to conduct studies to examine the effects of E₂, T-DM-1 alone and in combination of these cells. Both E₂ and T-DM-1 induced apoptosis. At the intermediate doses, the combination of E₂ plus T-DM-1 appeared to be more effective than either agent alone.

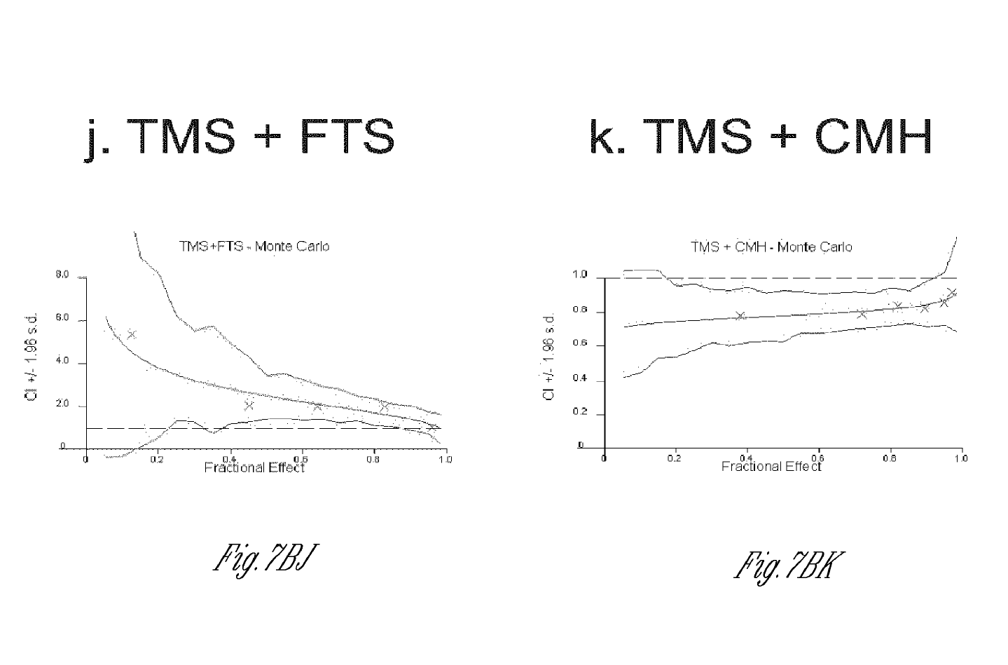

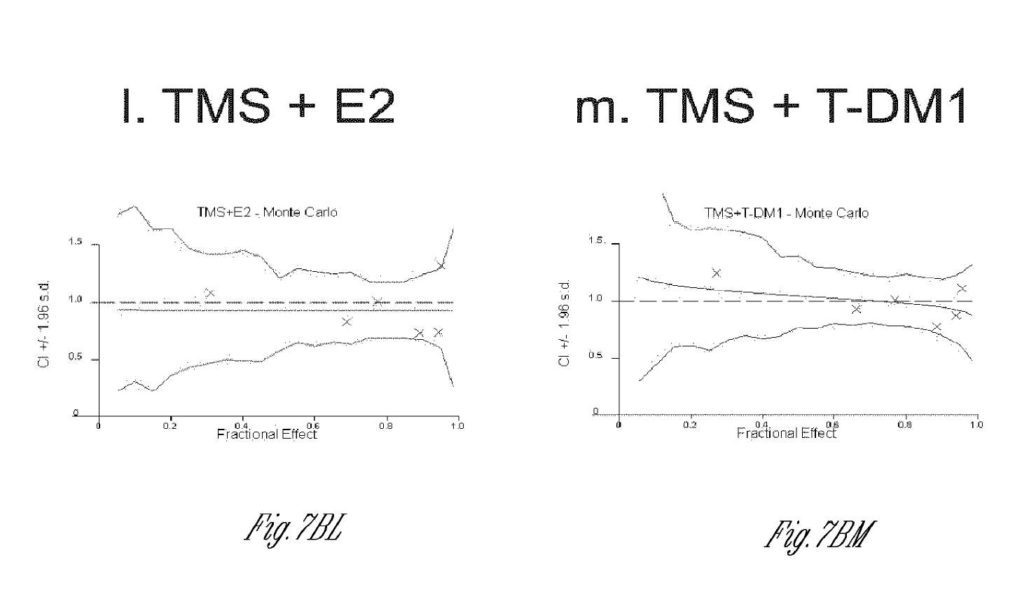

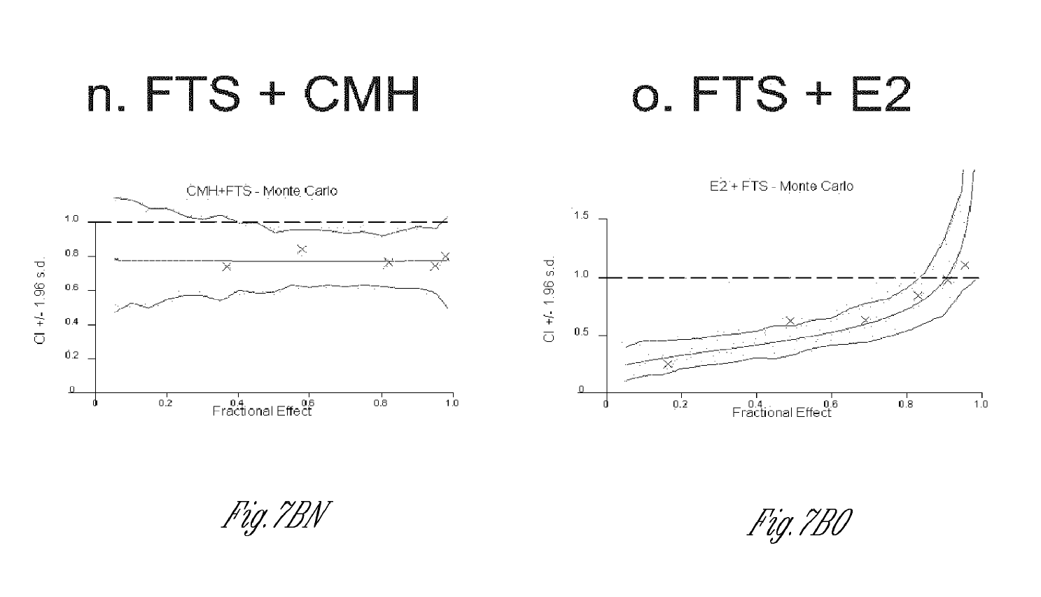

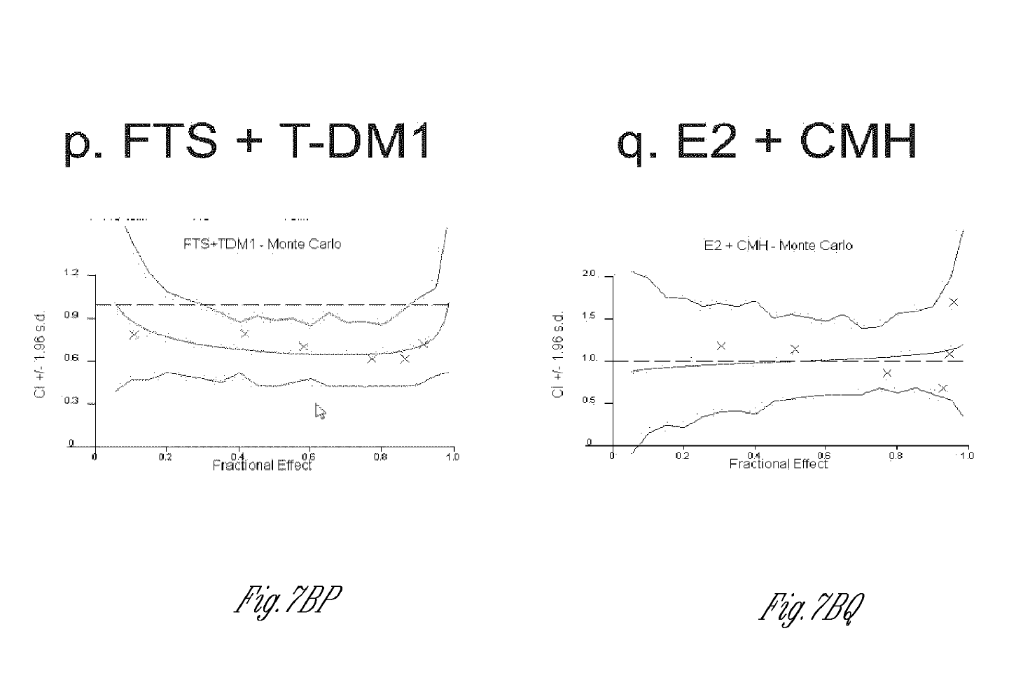

We examined some of these agents in both the non-adapted cell lines (See FIGS. 4, 6, 8) and adapted cell lines (See FIGS. 5, 7, 9). We treated breast cancer cells with increasing concentrations of the individual drugs followed by testing with their combinations. We determined the number of cells that were killed (fraction affected, fa) and the number of cells that were not affected by the drug (fraction unaffected, fu) (See FIG. 4). Then dose effect curves are transformed into their corresponding linear forms by the median-effect plot where y=log(fa/fu) vs. x=log (D)[8,34]. From the median effect plot the combination index (CI) can be determined. We have used the Monte Carlo option for plotting the CI graphs as this method calculates the mean and standard deviation values as well as displays the confidence intervals (See FIGS. 6, 7).

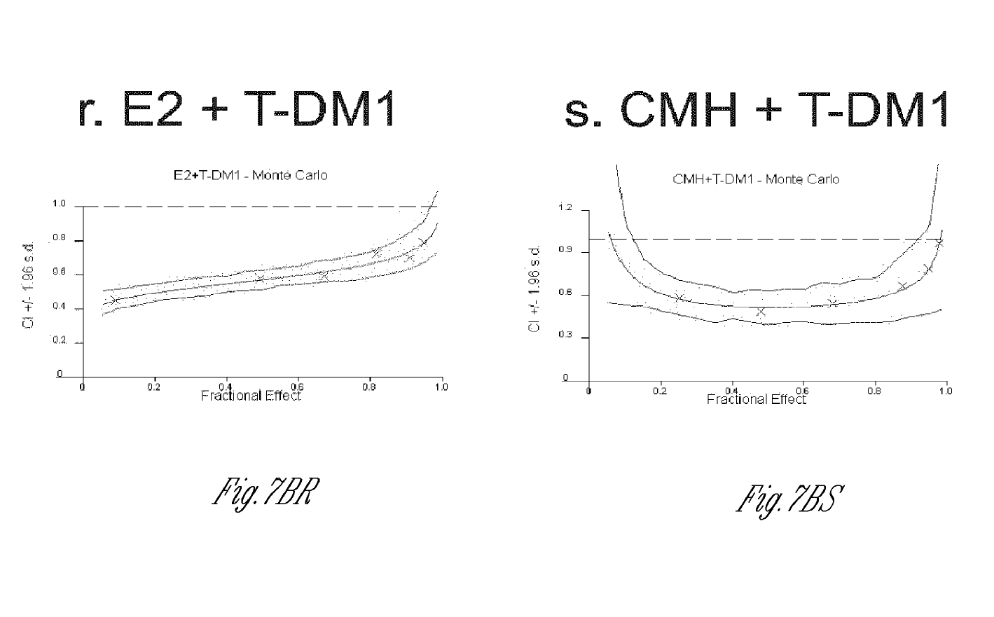

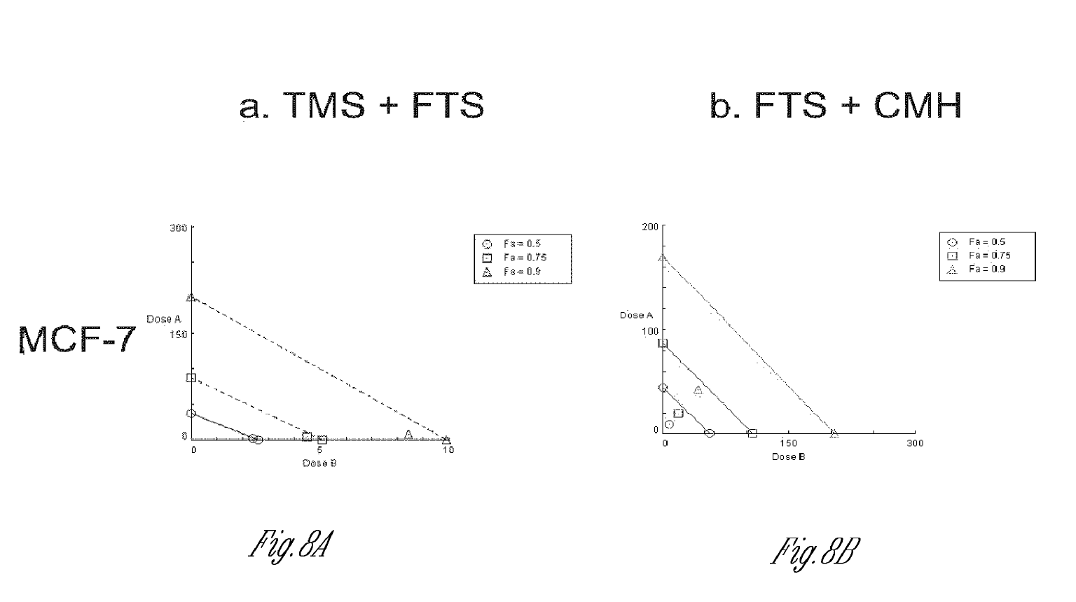

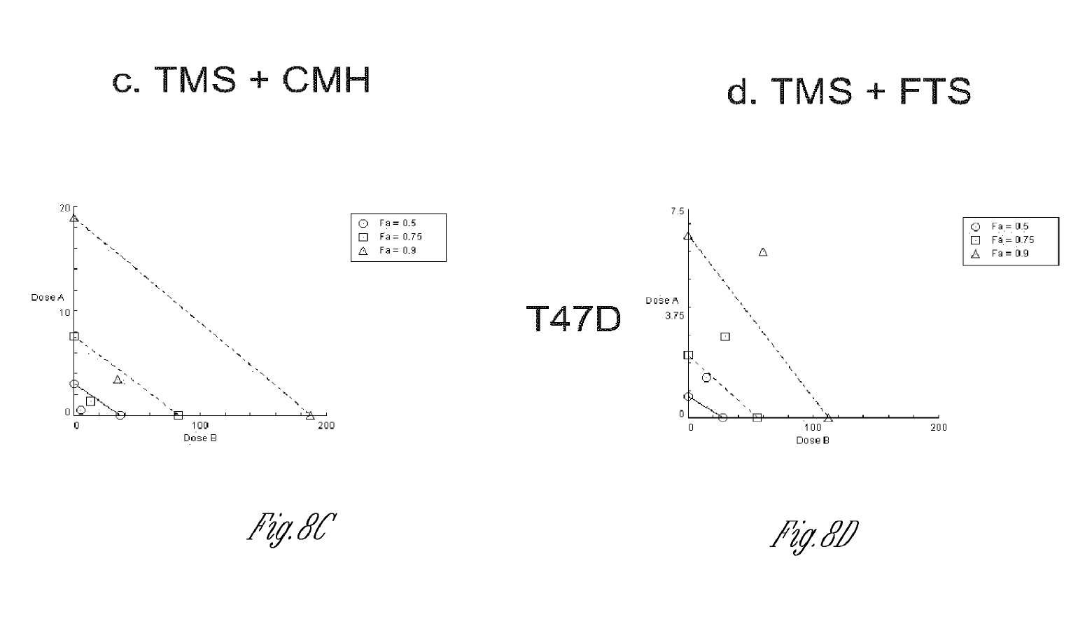

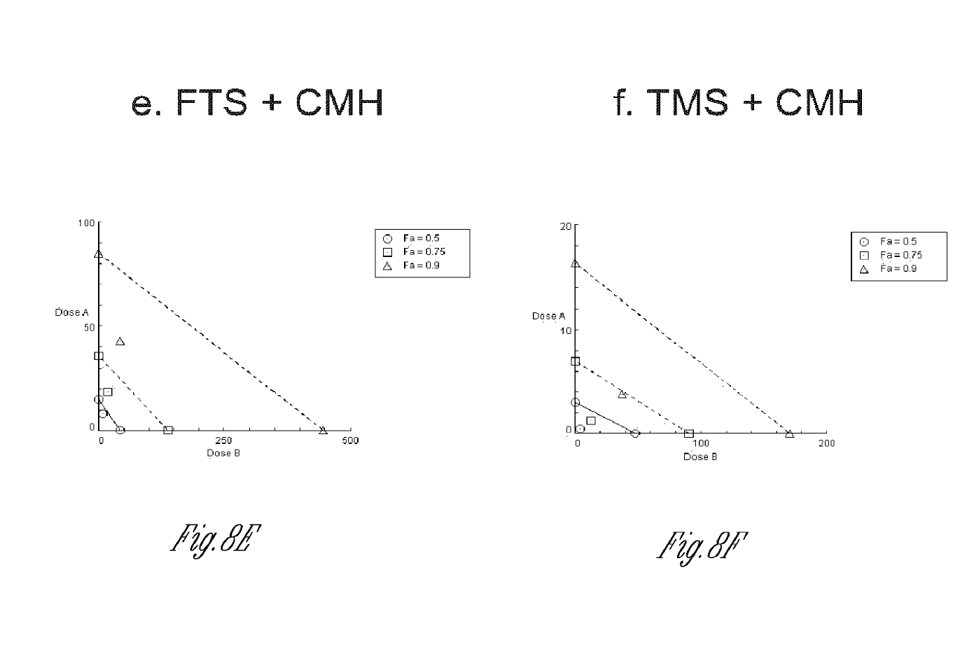

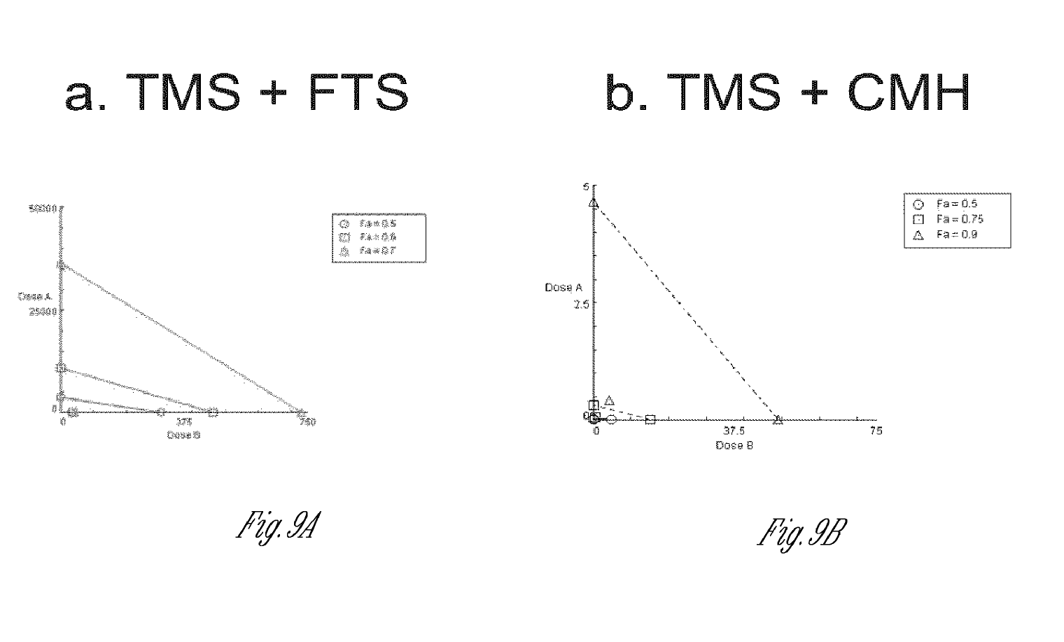

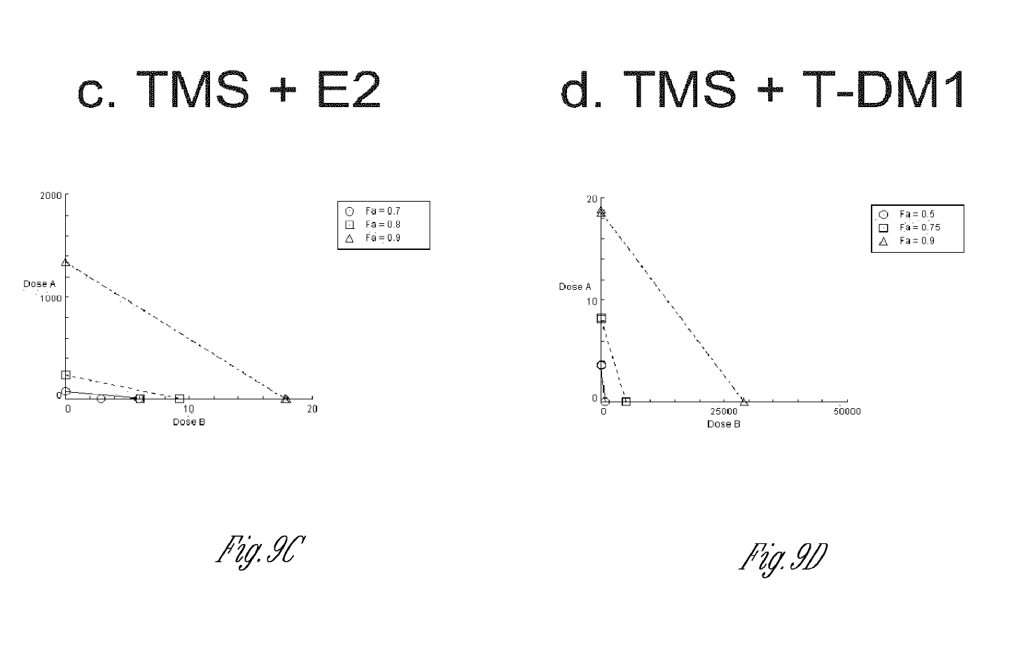

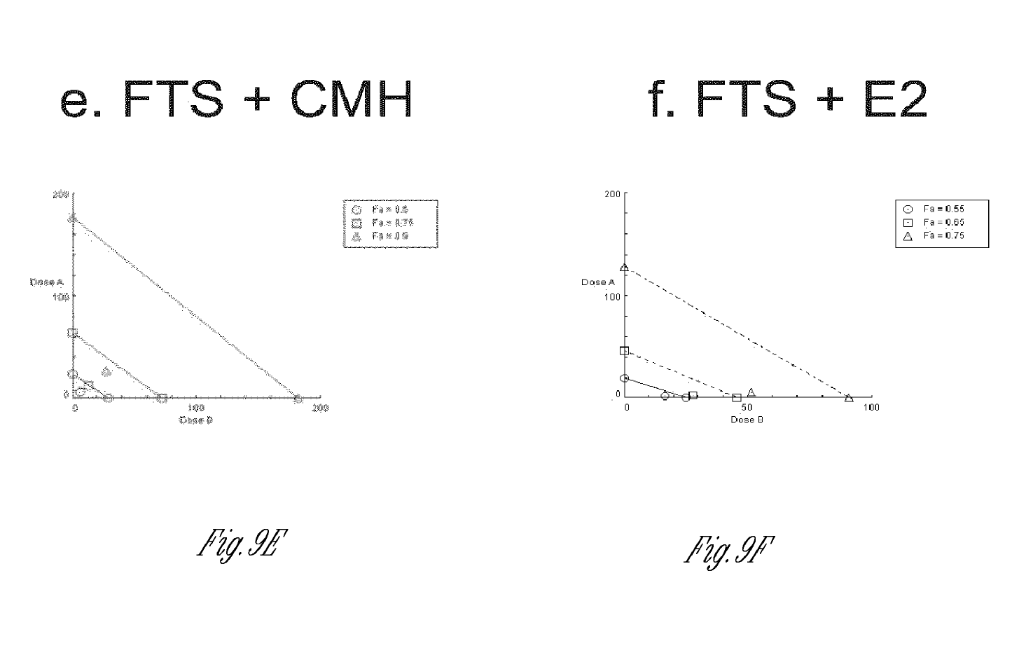

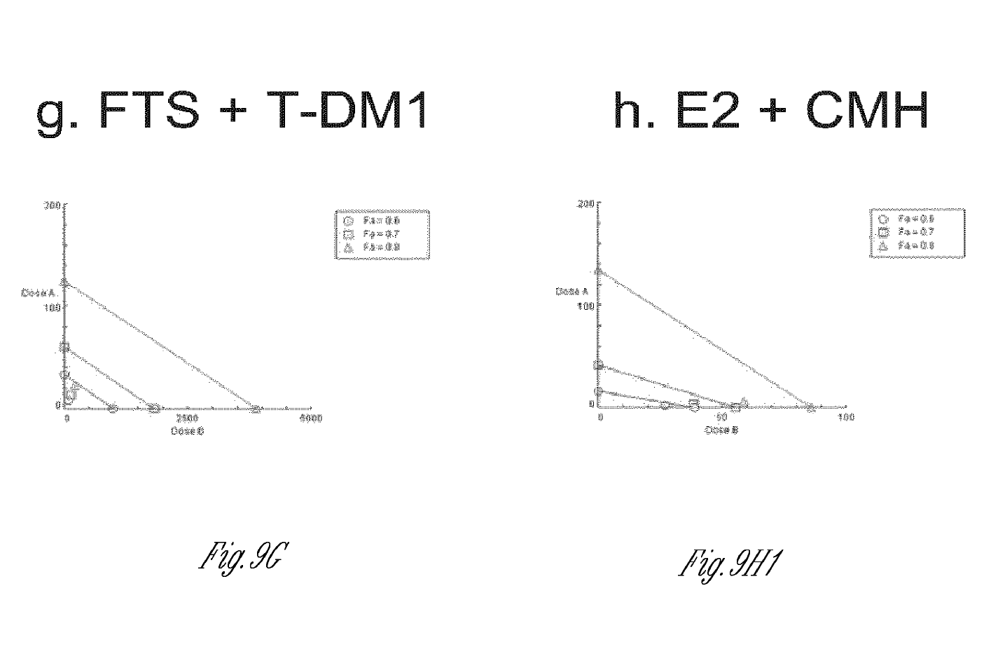

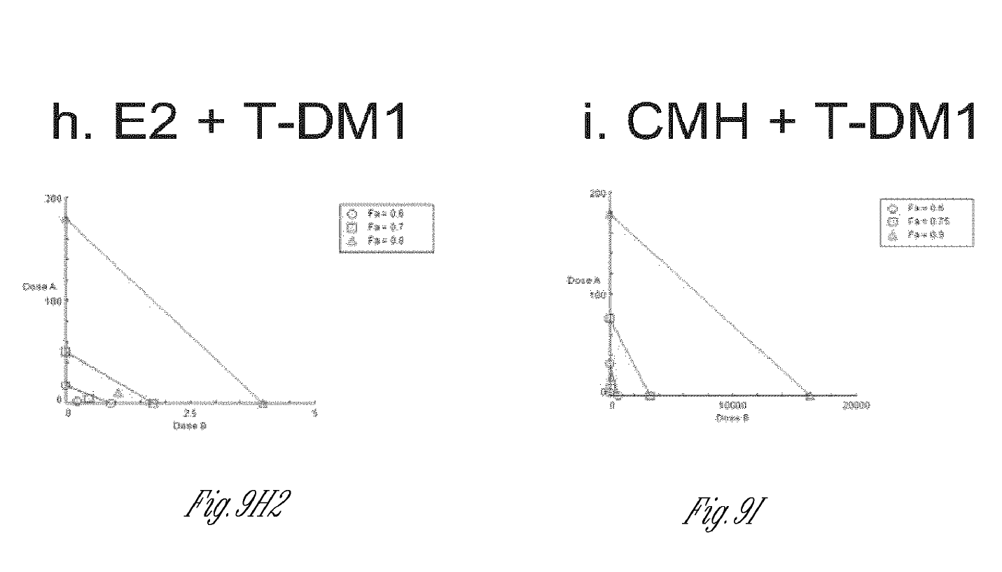

When the combination index is equivalent to one (CI=1), this means the two drugs work together in an additive manner. When the combination index is less than one (CI<1) the drugs are more effective than their individual sum and they display synergy. When the combination index is less than one (CI<1), then the two drugs together are less effective than when given individually and thus display antagonism. The effective dose one (D1) is then plotted on the x-axis and the effective dose two (D2) was plotted on the y-axis. The plotting of the effective dose (ED) can be is done at Fa equals 0.5, 0.75, 0.9 and 0.95. This generates the isobologram. We used these two methods, the combination index (FIGS. 6, 7) and the isobologram (FIGS. 8, 9), to determine if there is synergy when different combinations of agents are used. A summary of the results are shown in Table 1, above.

### Non-Adapted Cell Line Results

When we examined the non-adapted cells (FIGS. 4, 6, 8) we found that the combination index of TMS and FTS was additive in the MCF-7 cell line (FIG. 6a, FIG. 8a) and antagonistic in T47D cells (FIG. 6d, FIG. 8d). The combination of FTS and CMH displayed synergy for this combination in the MCF-7 cell line (FIG. 6b, 8b), but only mild synergy in the T47D cell line (FIG. 6e, 8e). The combination of TMS and CMH showed synergism in both the MCF-7 (FIGS. 6c, 8c) and T47D (FIG. 6f, 8f).

### Adapted Cell Line Results

Combinations with TMS varied. When TMS was combined with CMH there was synergy with the LTED cell line and mild synergy with the tamoxifen resistant cell line. When TMS was combined with either FTS, E2, or T-DM1 the combinations were additive in nature for both the LTED and TamR cells (FIG. 7 a, c-d, j, l-m). The combination of FTS with CMH showed mild synergy in both LTED and TamR cells (FIG. 7e, n). However, the combinations of FTS and T-DM1 and FTS and E2 showed stronger synergy in the LTED cells (FIG. 7f, g) compared to the tamoxifen resistant cell line (FIG. 7o, p) Combinations of E2 with CMH were additive in both cell lines (FIG. 7 e, q). The most efficacious combination was the combination of CMH and T-DM1 (FIG. 7i) in the LTED cell line. We observed a mild synergy with this combination in the tamoxifen resistant cell line (FIG. 7s). This may be due the fact the tamoxifen resistant cells have been cultured to a lesser extent in a low estrogen environment and therefore express a lower level of HER2.

Also see FIG. 9, which shows a graphical illustrations of an isobologram analysis of adapted cell lines.

### Summary of Results

Table 1, above, shows a summary of results, and combinations that resulted in synergy are highlighted. The strongest synergism we observed came from the combination of T-DM1 and CMH applied to the adapted LTED cell line. This is likely because this combination targets both the intrinsic mitochondrial death pathway as well as the extrinsic death receptor pathway, i.e., horizontal modulation has been achieved. T-DM 1 allows for targeting to the overexpressed HER2 on the surface of the LTED cells and DM1 agent invokes cell death through the intrinsic mitochondrial pathway. CMH modulates c-FLIP to activate the extrinsic death receptor pathway[14,15]. Both the potency and the targeting of T-DM 1 to HER2 are likely crucial for the synergy observed. All combinations with TMS that were tested were additive in nature (See FIGS. 6-9). FTS combinations were weaker in the adapted cell lines compared to the non-adapted LTED cell line (Compare FIG. 6b, d, e to FIG. 7e, f, g and also FIG. 8b, d, e to FIG. 9 e, f, g).

### Administration and Compositions

The present invention further provides adjunctive therapies that can be used in conjunction with the combination drug therapies. In various embodiments, combinations of pro-apoptotic anticancer drugs, such as combinations wherein different molecular mechanisms of apoptosis induction occur, may be used in combination with other therapeutic approaches as are well known in the art, including radiation therapy such as X-ray, gamma-ray, radionuclide emission, and subatomic particle exposure, brachytherapy, and use of additional anticancer agents that are either pro-apoptotic themselves or are cell growth inhibiting or cell reproduction inhibiting agents. Methods for evaluating these combinations and analyzing the results are known in the art.

The combination of effective medications to target multiple pathways opens new areas for developing pharmacotherapies for treating cancer. As disclosed herein, the combination therapies of the invention are based on targeting different/multiple pathways, including, but not limited to, inducing caspase-dependent death of cells, inhibiting cellular FLICE, activating caspases, including indirect activation of caspases, inhibiting HDAC3, HDAC6, and HDAC8, inducing caspase-independent death, modulating the mitochondrial death and Fas death receptor pathways, and disrupting microtubule structure.

The invention, provides in various embodiments methods for administration of a first pro-apoptotic anticancer agent “in conjunction with” a second pro-apoptotic anticancer agent. It will be appreciated by one of ordinary skill in the art that the two or more agent being administered in conjunction with each other do not necessarily have to be administered at the same time or in equal doses. In one aspect, the compounds being administered as part of the drug combination therapy are separately administered. In another aspect, a first compound is administered before a second compound is administered. In yet another aspect, a first compound and a second compound are administered nearly simultaneously. In a further aspect, the first compound is administered subsequent to administration of the second compound. Each of the agents can be administered multiple times, in doses, at frequencies of administration, and over periods of time that can be selected based upon the knowledge and skill of the medical practitioner.

The invention further provides pharmaceutical compositions comprising compounds of the invention. The pharmaceutical composition may comprise one or more compounds of the invention, and biologically active analogs, homologs, derivatives, modifications, and pharmaceutically acceptable salts thereof, and a pharmaceutically acceptable carrier. In one embodiment, the compounds are administered as a pharmaceutical composition.

The route of administration can vary depending on the type of compound being administered. In one aspect, the compounds are administered via routes such as oral, topical, rectal, intramuscular, intramucosal, intranasal, inhalation, ophthalmic, and intravenous.

The present invention further provides for administration of a compound of the invention as a controlled-release formulation.

In one embodiment, the results of treating a subject with a combination of two or more compounds are additive compared with the effects of using any of the compounds alone. In one aspect, the effects seen when using two or more compounds are greater than when using any of the compounds alone.

The present compositions can optionally comprise a suitable amount of a pharmaceutically acceptable vehicle so as to provide the form for proper administration to the patient.

The present compositions can also be administered to a subject in combination with behavioral therapy or interaction.

Included within the scope of this invention are the various individual anomers, diastereomers and enantiomers as well as mixtures thereof. In addition, the compounds of this invention also include any pharmaceutically acceptable salts, for example: alkali metal salts, such as sodium and potassium; ammonium salts; monoalkylammonium salts; dialkylammonium salts; trialkylammonium salts; tetraalkylammonium salts; and tromethamine salts. Hydrates and other solvates of the compounds are included within the scope of this invention.

If the initial dosage is not effective, then the dosage of one or more compounds of the combination therapy can be increased. If the initial dosage results in a more rapid weight loss than the above rate, the dosage of one or more of the at least two compounds can be reduced.

Pharmaceutically-acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amines, trisubstituted cycloalkyl amines, cycloalkenyl amines, di(cycloalkenyl) amines, tri(cycloalkenyl) amines, substituted cycloalkenyl amines, disubstituted cycloalkenyl amines, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. It should also be understood that other carboxylic acid derivatives would be useful in the practice of this invention, for example, carboxylic acid amides, including carboxamides, lower alkyl carboxamides, dialkyl carboxamides, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

In one embodiment, a composition of the invention may comprise one compound of the invention. In another embodiment, a composition of the invention may comprise more than one compound of the invention. In one embodiment, additional drugs or compounds useful for treating other disorders may be part of the composition. In one embodiment, a composition comprising only one compound of the invention may be administered at the same time as another composition comprising at least one other compound of the invention. In one embodiment, the different compositions may be administered at different times from one another. When a composition of the invention comprises only one compound of the invention, an additional composition comprising at least one additional compound must also be used.

The pharmaceutical compositions useful for practicing the invention may be, for example, administered to deliver a dose of between 1 ng/kg/day and 100 mg/kg/day.

Pharmaceutical compositions that are useful in the methods of the invention may be administered, for example, systemically in oral solid formulations, or as ophthalmic, suppository, aerosol, topical or other similar formulations. In addition to the appropriate compounds, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate compound, or an analog, modification, or derivative thereof according to the methods of the invention.

Compounds which are identified using any of the methods described herein may be formulated and administered to a subject for treatment of the diseases disclosed herein. One of ordinary skill in the art will recognize that these methods will be useful for other diseases, disorders, and conditions as well.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug, or may demonstrate increased palatability or be easier to formulate. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to provide the active moiety.

The invention encompasses the preparation and use of pharmaceutical compositions comprising a compound useful for treatment of the diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs, and birds including commercially relevant birds such as chickens, ducks, geese, and turkeys.

One type of administration encompassed by the methods of the invention is parenteral administration, which includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, and intrasternal injection, and kidney dialytic infusion techniques

Pharmaceutical compositions that are useful in the methods of the invention may be prepared, packaged, or sold in formulations suitable for oral, rectal, vaginal, parenteral, topical, pulmonary, intranasal, inhalation, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject, or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Lactulose can also be used as a freely erodible filler and is useful when the compounds of the invention are prepared in capsule form.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol; or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl para hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

In one aspect, a preparation in the form of a syrup or elixir or for administration in the form of drops may comprise active ingredients together with a sweetener, which is preferably calorie-free, and which may further include methylparaben or propylparaben as antiseptics, a flavoring and a suitable color.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of a dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil in water emulsion or a water-in-oil emulsion. The oily phase may be a vegetable oil such as olive or arachis oil, a mineral oil such as liquid paraffin, or a combination of these. Such compositions may further comprise one or more emulsifying agents including naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non irritating pharmaceutically acceptable excipient which is solid at ordinary room temperature (i.e. about 20° C.) and which is liquid at the rectal temperature of the subject (i.e. about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, and intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil in water or water in oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally, the propellant may constitute about 50% to about 99.9% (w/w) of the composition, and the active ingredient may constitute about 0.1% to about 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to about 500 micrometers. Such a formulation is administered in the manner in which snuff is taken, i.e., by rapid inhalation through the nasal passage from a container of the powder held close to the nares.

Formulations suitable for nasal administration may, for example, comprise from about as little as about 0.1% (w/w) and as much as about 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, comprise about 0.1% to about 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or atomized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1% to 1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

A pharmaceutical composition of the invention may be prepared, packaged, or sold in a formulation suitable for intramucosal administration. The present invention provides for intramucosal administration of compounds to allow passage or absorption of the compounds across mucosa. Such type of administration is useful for absorption orally (gingival, sublingual, buccal, etc.), rectally, vaginally, pulmonary, nasally, etc.

In some aspects, sublingual administration has an advantage for active ingredients which in some cases, when given orally, are subject to a substantial first pass metabolism and enzymatic degradation through the liver, resulting in rapid metabolization and a loss of therapeutic activity related to the activity of the liver enzymes that convert the molecule into inactive metabolites, or the activity of which is decreased because of this bioconversion.

In some cases, a sublingual route of administration is capable of producing a rapid onset of action due to the considerable permeability and vascularization of the buccal mucosa. Moreover, sublingual administration can also allow the administration of active ingredients which are not normally absorbed at the level of the stomach mucosa or digestive mucosa after oral administration, or alternatively which are partially or completely degraded in acidic medium after ingestion of, for example, a tablet.

Sublingual tablet preparation techniques known from the prior art are usually prepared by direct compression of a mixture of powders comprising the active ingredient and excipients for compression, such as diluents, binders, disintegrating agents and adjuvants. In an alternative method of preparation, the active ingredient and the compression excipients can be dry-granulated or wet-granulated beforehand. In one aspect, the active ingredient is distributed throughout the mass of the tablet. WO 00/16750 describes a tablet for sublingual use that disintegrates rapidly and comprises an ordered mixture in which the active ingredient is in the form of microparticles which adhere to the surface of water-soluble particles that are substantially greater in size, constituting a support for the active microparticles, the composition also comprising a mucoadhesive agent. WO 00/57858 describes a tablet for sublingual use, comprising an active ingredient combined with an effervescent system intended to promote absorption, and also a pH-modifier.

The compounds of the invention can be prepared in a formulation or pharmaceutical composition appropriate for administration that allows or enhances absorption across mucosa. Mucosal absorption enhancers include, but are not limited to, a bile salt, fatty acid, surfactant, or alcohol. In specific embodiments, the permeation enhancer can be sodium cholate, sodium dodecyl sulphate, sodium deoxycholate, taurodeoxycholate, sodium glycocholate, dimethylsulfoxide or ethanol. In a further embodiment, a compound of the invention can be formulated with a mucosal penetration enhancer to facilitate delivery of the compound. The formulation can also be prepared with pH optimized for solubility, drug stability, and absorption through mucosa such as nasal mucosa, oral mucosa, vaginal mucosa, respiratory, and intestinal mucosa.

To further enhance mucosal delivery of pharmaceutical agents within the invention, formulations comprising the active agent may also contain a hydrophilic low molecular weight compound as a base or excipient. Such hydrophilic low molecular weight compounds provide a passage medium through which a water-soluble active agent, such as a physiologically active peptide or protein, may diffuse through the base to the body surface where the active agent is absorbed. The hydrophilic low molecular weight compound optionally absorbs moisture from the mucosa or the administration atmosphere and dissolves the water-soluble active peptide. The molecular weight of the hydrophilic low molecular weight compound is generally not more than 10000 and preferably not more than 3000. Exemplary hydrophilic low molecular weight compounds include polyol compounds, such as oligo-, di- and monosaccharides such as sucrose, mannitol, lactose, L-arabinose, D-erythrose, D-ribose, D-xylose, D-mannose, D-galactose, lactulose, cellobiose, gentibiose, glycerin, and polyethylene glycol. Other examples of hydrophilic low molecular weight compounds useful as carriers within the invention include N-methylpyrrolidone, and alcohols (e.g., oligovinyl alcohol, ethanol, ethylene glycol, propylene glycol, etc.). These hydrophilic low molecular weight compounds can be used alone or in combination with one another or with other active or inactive components of the intranasal formulation.

When a controlled-release pharmaceutical preparation of the present invention further contains a hydrophilic base, many options are available for inclusion. Hydrophilic polymers such as a polyethylene glycol and polyvinyl pyrrolidone, sugar alcohols such as D-sorbitol and xylitol, saccharides such as sucrose, maltose, lactulose, D-fructose, dextran, and glucose, surfactants such as polyoxyethylene-hydrogenated castor oil, polyoxyethylene polyoxypropylene glycol, and polyoxyethylene sorbitan higher fatty acid esters, salts such as sodium chloride and magnesium chloride, organic acids such as citric acid and tartaric acid, amino acids such as glycine, beta-alanine, and lysine hydrochloride, and aminosaccharides such as meglumine are given as examples of the hydrophilic base. Polyethylene glycol, sucrose, and polyvinyl pyrrolidone are preferred and polyethylene glycol are further preferred. One or a combination of two or more hydrophilic bases can be used in the present invention.

The present invention contemplates pulmonary, nasal, or oral administration through an inhaler. In one embodiment, delivery from an inhaler can be a metered dose.

An inhaler is a device for patient self-administration of at least one compound of the invention comprising a spray inhaler (e.g., a nasal, oral, or pulmonary spray inhaler) containing an aerosol spray formulation of at least one compound of the invention and a pharmaceutically acceptable dispersant. In one aspect, the device is metered to disperse an amount of the aerosol formulation by forming a spray that contains a dose of at least one compound of the invention effective to treat a disease or disorder encompassed by the invention. The dispersant may be a surfactant, such as, but not limited to, polyoxyethylene fatty acid esters, polyoxyethylene fatty acid alcohols, and polyoxyethylene sorbitan fatty acid esters. Phospholipid-based surfactants also may be used.

In other embodiments, the aerosol formulation is provided as a dry powder aerosol formulation in which a compound of the invention is present as a finely divided powder. The dry powder formulation can further comprise a bulking agent, such as, but not limited to, lactose, sorbitol, sucrose, and mannitol.

In another specific embodiment, the aerosol formulation is a liquid aerosol formulation further comprising a pharmaceutically acceptable diluent, such as, but not limited to, sterile water, saline, buffered saline and dextrose solution.

In further embodiments, the aerosol formulation further comprises at least one additional compound of the invention in a concentration such that the metered amount of the aerosol formulation dispersed by the device contains a dose of the additional compound in a metered amount that is effective to ameliorate the symptoms of disease or disorder disclosed herein when used in combination with at least a first or second compound of the invention.

Thus, the invention provides a self administration method for outpatient treatment of an addiction related disease or disorder such as an alcohol-related disease or disorder. Such administration may be used in a hospital, in a medical office, or outside a hospital or medical office by non-medical personnel for self administration.

Compounds of the invention will be prepared in a formulation or pharmaceutical composition appropriate for nasal administration. In a further embodiment, the compounds of the invention can be formulated with a mucosal penetration enhancer to facilitate delivery of the drug. The formulation can also be prepared with pH optimized for solubility, drug stability, absorption through nasal mucosa, and other considerations.

Capsules, blisters, and cartridges for use in an inhaler or insufflator may be formulated to contain a powder mix of the pharmaceutical compositions provided herein; a suitable powder base, such as lactose or starch; and a performance modifier, such as l-leucine, mannitol, or magnesium stearate. The lactose may be anhydrous or in the form of the monohydrate. Other suitable excipients include dextran, glucose, maltose, sorbitol, xylitol, fructose, sucrose, and trehalose. The pharmaceutical compositions provided herein for inhaled/intranasal administration may further comprise a suitable flavor, such as menthol and levomenthol, or sweeteners, such as saccharin or saccharin sodium.

For administration by inhalation, the compounds for use according to the methods of the invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of e.g., gelatin for use in an inhaler or insufflator may be formulated containing a powder mix of the drugs and a suitable powder base such as lactose or starch.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically, dosages of the compounds of the invention which may be administered to an animal, preferably a human, range in amount from about 1.0 μg to about 100 g per kilogram of body weight of the animal. The precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The compounds may be administered to a subject as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

The invention also includes a kit comprising the compounds of the invention and an instructional material that describes administration of the compounds. In another embodiment, this kit comprises a (preferably sterile) solvent suitable for dissolving or suspending the composition of the invention prior to administering the compound to the mammal.

As used herein, an “instructional material” includes a publication, a recording, a diagram, or any other medium of expression that can be used to communicate the usefulness of the compounds of the invention in the kit for effecting alleviation of the various diseases or disorders recited herein. Optionally, or alternately, the instructional material may describe one or more methods of alleviating the diseases or disorders. The instructional material of the kit of the invention may, for example, be affixed to a container that contains a compound of the invention or be shipped together with a container that contains the compounds. Alternatively, the instructional material may be shipped separately from the container with the intention that the instructional material and the compound be used cooperatively by the recipient.

Without further description, it is believed that one of ordinary skill in the art can, using the preceding description and the following illustrative examples, make and utilize the compounds of the present invention and practice the claimed methods. The following working examples, therefore, specifically point out the preferred embodiments of the present invention, and are not to be construed as limiting in any way the remainder of the disclosure.

### EXAMPLES

### Materials and Methods

### Drugs and Chemicals

S-trans, trans-Farnesylthiosalicyclic acid (FTS, Salirasib), a known Ras inhibitor, was obtained from Concordia Pharmaceuticals, Inc. Ft. Lauderdale, Fla. 4-(4-Chloro-2-methylphenoxy)-N-hydroxybutanamide (CMH) (5809354) and its inactive analog 4-(4-chloro-2-methylphenoxy)-N-(3-ethoxypropyl) butanamide (CMB) (6094911) were purchased from ChemBridge Corporation (San Diego, Calif.). TMS was synthesized as described previously (Kim S, Ko H, Park J E, Jung S, Lee S K, Chun Y J: Design, synthesis, and discovery of novel trans-stilbene analogues as potent and selective human cytochrome P450 1B1 inhibitors. J Med Chem 2002, 45: 160-164). 17β-Estradiol was obtained from Steraloids, Inc. (Newport, R.I.). T-DM1 was a gift from Genentech, San Francisco, Calif. Tamoxifen and δ-Tocotrienol were purchased from Sigma-Aldrich Co. (St. Louis, Mo.).

### Cell Culture Conditions

Parental MCF-7 were grown in IMEM with 5% FBS. T47D cells were grown in RPMI160 with 10% FBS. Tamoxifen-resistant postmenopausal cells were grown in phenol-free IMEM with 5% DCC and treated with tamoxifen (10⁻⁷ M) for more than one year[5]. Long-term estrogen deprived cells were grown in phenol free IMEM with 5% DCC[6]. LTEDaro cells, which overexpress aromatase, were a kind gift from Dr. Chen[7] and were grown in phenol-red free MEM, supplemented with 10% DCC, 100 mg/L sodium pyruvate, 2 mM L-glutamine, and 200 mg/L G418.

### Growth Inhibition and Drug Interaction Assays

Cells were plated in six-well plates at a density of 60,000 cells per well. Two days later, the cells were treated in triplicate as described in the descriptions of the figures. At the end of treatment, cells were rinsed twice with saline. Nuclei were prepared by sequential addition of 1 mL HEPES-MgCl2 solution (0.01 mol/L HEPES and 1.5 mmol/L MgCl2) and 0.1 mL ZAP solution [0.13 mol/L ethylhexadecyldimethylammonium bromide in 3% glacial acetic acid (v/v)] and were counted using a Coulter counter (BeckmanCoulter, Inc., Fullerton, Calif.). Dose response curves were obtained from triplicate samples and the median effective dose, Dₘ, was computed using Compusyn software[8,9]. The combination index and values with a mean and standard deviation were calculated using the Monte Carlo simulation using the computer software CalcuSyn from Biosoft (Cambridge, U.K.) [10].

### Immunoprecipitation

Cells grown in 100 mm dishes were washed with cold PBS and extracted with 1 ml lysis buffer (50 mM Tris-HCl, 150 mM NaCl, 5 mM EDTA, 25 mM NaF, 2 mM NaVO₄, 5% glycerol, 1% Triton X-100, 10 μg/ml leupeptin, aprotinin, and pepstatin). Samples were incubated on ice for 30 min, sonicated, and centrifuged at 14,000 rpm for 10 min at 4° C. Supernatants containing 0.5 mg total protein were incubated with antibody against the target protein at 4° C. overnight before addition of 40 μl Protein G beads (Invitrogen) and continued incubation at 4° C. for 2 h. The protein G beads with immunocomplex were centrifuged at 14,000 rpm for 20 sec. The supernatant was carefully removed. The beads were washed twice with 1 ml buffer II (20 mM MOPS, 2 mM EGTA, 5 mM EDTA, 25 mM NaF, 40 mM β-glycerophosphate, 10 mM sodium pyrophosphate, 2 mM NaVO₄, 0.5% Triton X-100, 1 mM PMSF, 10 μg/ml leupeptin, aprotinin, and pepstatin) and then boiled in 50 μl 2× Laemmli's buffer. The samples were subjected to electrophoresis in 10% SDS polyacrylamide gel followed by immunoblotting.

### Combination Index and Isobologram Analysis

The nature of the interaction between the agents was evaluated by the combination index method of Chou and Talalay [8,9]. This method is based on the median effect principle:

fₐ/fᵤ(D/Dₘ)^{m} (1)

where D is the dose and Dₘ is the dose that yields 50% growth inhibition, fₐ is the cell fraction affected by dose D, and fᵤ is the unaffected fraction, and m is the coefficient that defines the sigmoidicity of the dose effect curve. This relationship and the law of mass action lead to a generalized equation for the interaction of multiple inhibitors:

(fₐ)_{A,B}/(fᵤ)_{A,B}=(fₐ)_{A}/(fᵤ)_{B}+(fₐ)_{B}/(fᵤ)_{B}+α(fₐ)_{A}(fₐ)_{B}/(fᵤ)_{A}(fᵤ)_{B} (2)

Where (fₐ)_{A}, (fᵤ)_{B} and (fₐ)_{A,B} are the fraction affected by agents A and B alone and in combination. From equations 1 and 2 the combination index (CI) can be derived as

CI=(D)_{A}/(Dₓ)_{A}+(D)_{B}/(Dₓ)_{B}+α(D)_{A}(D)_{B}/(Dₓ)_{A}(Dₓ)_{B} (3)

Where D is the dose that yields x % growth inhibition and α=0 for mutually exclusive drugs and α=1 for mutually non-exclusive drugs. Synergy as calculated and defined by the CalcuSyn software as a CI<1; additivity is CI=1 and antagonism is CI>1[10].

### Statistical Analysis

The mean and standard deviation values of the combination index were calculated using the Monte Carlo algorithm within the CalcuSyn program[10].

### Embodiments of the Invention

1. A method of treating a cancer, comprising administering to a patient afflicted therewith an effective amount of an immunoconjugate comprising a monoclonal antibody moiety and a first pro-apoptotic drug moiety linked thereto; and administering to the patient an effective amount of a second pro-apoptotic drug.
2. The method of embodiment 1, wherein the first pro-apoptotic drug moiety is covalently linked to the monoclonal antibody moiety.
3. The method of embodiment 1 or 2, wherein the cancer is breast cancer.
4. The method of embodiment 3, wherein the breast cancer is aromatase-resistant breast cancer.
5. The method of embodiment 3 wherein the breast cancer is tamoxifen-resistant breast cancer.
6. The method of embodiment 3, wherein the breast cancer is ER+ hormone refractory breast cancer.
7. The method of embodiment 3, wherein the breast cancer is HER2 positive breast cancer.
8. The method of embodiment 5, wherein the breast cancer is HER2 positive breast cancer.
9. The method of embodiment 3, wherein the breast cancer comprises cancer cells in which HER2 expression is up-regulated.
10. The method of any one of embodiments 1-9, wherein the immunoconjugate binds to HER2.
11. The method of embodiment 9 wherein the monoclonal antibody moiety is trastuzumab.
12. The method of any one of embodiments 1-11 wherein the first pro-apoptotic drug moiety is a microtubule depolymerization agent.
13. The method of embodiment 12 wherein the first pro-apoptotic drug moiety is a maytansinoid or an auristatin.
14. The method of any one of embodiments 1-12 wherein the immunoconjugate is trastuzumab covalently coupled via a linker with a maytansinoid pro-apoptotic drug moiety.
15. The method of embodiment 14 wherein the immunoconjugate is T-DM1.
16. The method of any one of embodiments 1-15, wherein the second pro-apoptotic drug exerts cytotoxicity by a molecular mechanism other than the molecular mechanism of cytotoxicity exerted by the first pro-apoptotic drug moiety.
17. The method of any one of embodiments 1-16 wherein administering the immunoconjugate and the second pro-apoptotic drug has a synergistic effect.
18. The method of embodiment 16, wherein the second pro-apoptotic drug is a drug that induces apoptosis via an extrinsic pathway.
19. The method of embodiment 18, wherein the second pro-apoptotic drug induces apoptosis via a Fas pathway.
20. The method of embodiment 18, wherein the second pro-apoptotic drug induces apoptosis via a c-FLIP pathway.
21. The method of embodiment 18 wherein the second pro-apoptotic drug is CMH, E2, or δ-tocotrienol.
22. The method of embodiment 16, wherein the second pro-apoptotic drug is a drug that induces apoptosis via an intrinsic pathway.
23. The method of embodiment 22, wherein the second pro-apoptotic drug induces apoptosis via a caspase-independent pathway.
24. The method of embodiment 22, wherein the second pro-apoptotic drug induces apoptosis via a caspase-dependent pathway.
25. The method of embodiment 22 wherein the second pro-apoptotic drug is E2, FTS, or δ-tocotrienol.
26. The method of any one of embodiments 1-25, wherein the second pro-apoptotic anticancer drug is FTS, CMH, E2, TMS, δ-tocotrienol, or curcumin.
27. The method of any one of embodiments 1-26, wherein the immunoconjugate is T-DM1 and the second pro-apoptotic drug is FTS, CMH, E2, TMS, δ-tocotrienol, or curcumin.
28. The method of embodiment 27 wherein the second pro-apoptotic drug is E2, FTS, δ-tocotrienol, or TMS.
29. The method of embodiment 27 wherein the second pro-apoptotic drug is FTS.
30. The method of embodiment 27 wherein administering the immunoconjugate and the second pro-apoptotic drug has a synergistic effect.
31. The method of any one of embodiments 1-30, wherein the linker moiety is cleaved in vivo within a HER2-resistant breast cancer cell following administration of the immunoconjugate.
32. The method of embodiment 1 comprising treatment of an aromatase-resistant breast cancer in a patient afflicted therewith, comprising administering to the patent an effective amount of T-DM1 in conjunction with an effective amount of FTS, CMH, E2, TMS, δ-tocotrienol, or curcumin, or any combination thereof.
33. The method of any one of embodiments 1-32 wherein the method is an adjuvant therapy.
34. The method of any one of embodiments 1-32 wherein the method is a first-line therapy.
35. The method of any one of embodiments 1-32 wherein the method is a second-line therapy.
36. The method of any one of embodiments 1-35 wherein the immunoconjugate and the second pro-apoptotic drug are administered as a combined formulation or by alternation.
37. The method of any one of embodiments 1-36 further comprising administering to the patient an additional anticancer drug, wherein optionally the additional anticancer drug exerts an effect via a molecular mechanism different from the molecular mechanism of the first pro-apoptotic anticancer drug moiety and different from the molecular mechanism of the second pro-apoptotic anticancer drug; or administration to the patient of ionizing radiation comprising X-rays, gamma-rays, emissions of radionuclides, or subatomic particles; or any combination thereof.
38. A therapeutic composition comprising (a) an immunoconjugate comprising a monoclonal antibody moiety linked to a first pro-apoptotic drug moiety, and (b) a second pro-apoptotic drug.
39. The composition of embodiment 38 wherein the covalent immunoconjugate is T-DM1.
40. The composition of embodiment 38 wherein the second pro-apoptotic anticancer drug is FTS, CMH, E2, TMS, δ-tocotrienol, or curcumin.

The disclosures of each and every patent, patent application, and publication cited herein are hereby incorporated by reference herein in their entirety.

Headings are included herein for reference and to aid in locating certain sections. These headings are not intended to limit the scope of the concepts described therein under, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

### BIBLIOGRAPHY

1. Jemal A, Siegel R, Xu J, Ward E: Cancer statistics, 2010. CA Cancer J Clin 2010, 60: 277-300.
2. Malvezzi M, Arfe A, Bertuccio P, Levi F, La V C, Negri E: European cancer mortality predictions for the year 2011. Ann Oncol 2011.
3. Musgrove E A, Sutherland R L: Biological determinants of endocrine resistance in breast cancer. Nat Rev Cancer 2009, 9: 631-643.
4. Kim S, Ko H, Park J E, Jung S, Lee S K, Chun Y J (2002) Design, synthesis, and discovery of novel trans-stilbene analogues as potent and selective human cytochrome P450 1B1 inhibitors. J Med Chem 45:160-164
5. Fan P, Yue W, Wang J P, Aiyar S, Li Y, Kim T H, Santen R J (2009) Mechanisms of resistance to structurally diverse antiestrogens differ under premenopausal and postmenopausal conditions: evidence from in vitro breast cancer cell models. Endocrinology 150:2036-2045
6. Jeng M H, Shupnik M A, Bender T P, Westin E H, Bandyopadhyay D, Kumar R, Masamura S, Santen R J (1998) Estrogen receptor expression and function in long-term estrogen-deprived human breast cancer cells. Endocrinology 139:4164-4174
7. Chen S, Masri S, Hong Y, Wang X, Phung S, Yuan Y C, Wu X (2007) New experimental models for aromatase inhibitor resistance. J Steroid Biochem Mol Biol 106:8-15
8. Chou T C (2006) Theoretical basis, experimental design, and computerized simulation of synergism and antagonism in drug combination studies. Pharmacol Rev 58:621-681
9. Chou T C (2008) Preclinical versus clinical drug combination studies. Leuk Lymphoma 49:2059-2080
10. Chang T T, Chou T C (2000) Rational approach to the clinical protocol design for drug combinations: a review. Acta Paediatr Taiwan 41:294-302
11. Blum R, Jacob-Hirsch J, Rechavi G, Kloog Y (2006) Suppression of survivin expression in glioblastoma cells by the Ras inhibitor farnesylthiosalicylic acid promotes caspase-dependent apoptosis. Mol Cancer Ther 5:2337-2347
12. Charette N, De S C, Lannoy V, Horsmans Y, Leclercq I, Starkel P (2010) Salirasib inhibits the growth of hepatocarcinoma cell lines in vitro and tumor growth in vivo through ras and mTOR inhibition. Mol Cancer 9:256
13. Santen R J, Lynch A R, Neal L R, McPherson R A, Yue W (2006) Farnesylthiosalicylic acid: inhibition of proliferation and enhancement of apoptosis of hormone-dependent breast cancer cells. Anticancer Drugs 17:33-40
14. Bijangi-Vishehsaraei K, Saadatzadeh M R, Huang S, Murphy M P, Safa A R (2010) 4-(4-Chloro-2-methylphenoxy)-N-hydroxybutanamide (CMH) targets mRNA of the c-FLIP variants and induces apoptosis in MCF-7 human breast cancer cells. Mol Cell Biochem 342:133-142
15. Wood T E, Dalili S, Simpson C D, Sukhai M A, Hurren R, Anyiwe K, Mao X, Suarez S F, Gronda M, Eberhard Y, MacLean N, Ketela T, Reed J C, Moffat J, Minden M D, Batey R A, Schimmer A D (2010) Selective inhibition of histone deacetylases sensitizes malignant cells to death receptor ligands. Mol Cancer Ther 9:246-256
16. Aiyar S E, Park H, Aldo P B, Mor G, Gildea J J, Miller A L, Thompson E B, Castle J D, Kim S, Santen R J (2010) TMS, a chemically modified herbal derivative of resveratrol, induces cell death by targeting Bax. Breast Cancer Res Treat 124:265-277
17. Park H, Aiyar S E, Fan P, Wang J, Yue W, Okouneva T, Cox C, Jordan M A, Demers L, Cho H, Kim S, Song R X, Santen R J (2007) Effects of tetramethoxystilbene on hormone-resistant breast cancer cells: biological and biochemical mechanisms of action. Cancer Res 67:5717-5726
18. Lewis J S, Meeke K, Osipo C, Ross E A, Kidawi N, Li T, Bell E, Chandel N S, Jordan V C (2005) Intrinsic mechanism of estradiol-induced apoptosis in breast cancer cells resistant to estrogen deprivation. J Natl Cancer Inst 97:1746-1759
19. Song R X, Mor G, Naftolin F, McPherson R A, Song J, Zhang Z, Yue W, Wang J, Santen R J (2001) Effect of long-term estrogen deprivation on apoptotic responses of breast cancer cells to 17beta-estradiol. J Natl Cancer Inst 93:1714-1723
20. Jordan V C, Lewis-Wambi J S, Patel R R, Kim H, Ariazi E A (2009) New hypotheses and opportunities in endocrine therapy: amplification of oestrogen-induced apoptosis. Breast 18 Suppl 3:S10-S17
21. Santen R J, Allred D C (2007) The estrogen paradox. Nat Clin Pract Endocrinol Metab 3:496-497
22. Song R X, Santen R J (2003) Apoptotic action of estrogen. Apoptosis 8:55-60
23. Lewis J S, Meeke K, Osipo C, Ross E A, Kidawi N, Li T, Bell E, Chandel N S, Jordan V C (2007) Intrinsic mechanism of estradiol-induced apoptosis in breast cancer cells resistant to estrogen deprivation. J Natl Cancer Inst 97:1746-1759
24. Shim W S, Conaway M, Masamura S, Yue W, Wang J P, Kmar R, Santen R J (2000) Estradiol hypersensitivity and mitogen-activated protein kinase expression in long-term estrogen deprived human breast cancer cells in vivo. Endocrinology 141:396-405
25. Ingle J N, Ahmann D L, Green S J, Edmonson J H, Bisel H F, Kvols L K, Nichols W C, Creagan E T, Hahn R G, Rubin J, Frytak S (1981) Randomized clinical trial of diethylstilbestrol versus tamoxifen in postmenopausal women with advanced breast cancer. N Engl J Med 304:16-21
26. Oroudjev E, Lopus M, Wilson L, Audette C, Provenzano C, Erickson H, Kovtun Y, Chari R, Jordan M A (2010) Maytansinoid-antibody conjugates induce mitotic arrest by suppressing microtubule dynamic instability. Mol Cancer Ther 9:2700-2713
27. Esteve M A, Cane M, Braguer D (2007) Microtubules in apoptosis induction: are they necessary? Curr Cancer Drug Targets 7:713-729
28. Jordan M A, Wilson L (2004) Microtubules as a target for anticancer drugs. Nat Rev Cancer 4:253-265
29. Jumbe N L, Xin Y, Leipold D D, Crocker L, Dugger D, Mai E, Sliwkowski M X, Fielder P J, Tibbitts J (2010) Modeling the efficacy of trastuzumab-DM1, an antibody drug conjugate, in mice. J Pharmacokinet Pharmacodyn 37:221-242
30. Burris H A, III, Rugo H S, Vukelja S J, Vogel C L, Borson R A, Limentani S, Tan-Chiu E, Krop I E, Michaelson R A, Girish S, Amler L, Zheng M, Chu Y W, Klencke B, O'Shaughnessy J A (2011) Phase II study of the antibody drug conjugate trastuzumab-DM 1 for the treatment of human epidermal growth factor receptor 2 (HER2)-positive breast cancer after prior HER2-directed therapy. J Clin Oncol 29:398-405
31. Sabnis G, Brodie A (2010) Understanding resistance to endocrine agents: molecular mechanisms and potential for intervention. Clin Breast Cancer 10:E6-E15
32. Sabnis G, Brodie A (2010) Adaptive changes results in activation of alternate signaling pathways and resistance to aromatase inhibitor resistance. Mol Cell Endocrinol
33. Flageng M H, Moi L L, Dixon J M, Geisler J, Lien E A, Miller W R, Lonning P E, Mellgren G (2009) Nuclear receptor co-activators and HER2/neu are upregulated in breast cancer patients during neo-adjuvant treatment with aromatase inhibitors. Br J Cancer 101:1253-1260
34. Chou T C, Talalay P (1984) Quantitative analysis of dose-effect relationships: the combined effects of multiple drugs or enzyme inhibitors. Adv Enzyme Regul 22:27-55

1. A method of treating a cancer, comprising administering to a patient afflicted therewith an effective amount of an immunoconjugate comprising a monoclonal antibody moiety and a first pro-apoptotic drug moiety linked thereto; and administering to the patient an effective amount of a second pro-apoptotic drug.

2. The method of claim 1 wherein the first pro-apoptotic drug moiety is covalently linked to the monoclonal antibody moiety.

3. The method of claim 1 wherein the cancer is breast cancer.

4. The method of claim 3 wherein the breast cancer is aromatase-resistant breast cancer.

5. The method of claim 3 wherein the breast cancer is tamoxifen-resistant breast cancer.

6. The method of claim 3 wherein the breast cancer is ER+ hormone refractory breast cancer.

7. The method of claim 3 wherein the breast cancer is HER2 positive breast cancer.

8. The method of claim 5 wherein the breast cancer is HER2 positive breast cancer.

9. The method of claim 7 wherein the monoclonal antibody moiety binds to HER2.

10. The method of claim 9 wherein the monoclonal antibody moiety is trastuzumab.

11. The method of claim 1 wherein the first pro-apoptotic drug moiety is a microtubule depolymerization agent.

12. The method of claim 11 wherein the first pro-apoptotic drug moiety is a maytansinoid or an auristatin.

13. The method of claim 11, wherein the monoclonal antibody moiety binds to HER2.

14. The method of claim 12 wherein the immunoconjugate is T-DM1.

15. The method of claim 1, wherein the second pro-apoptotic drug exerts cytotoxicity by a molecular mechanism other than the molecular mechanism of cytotoxicity exerted by the first pro-apoptotic drug moiety.

16. The method of claim 1 wherein administering the immunoconjugate and the second pro-apoptotic drug has a synergistic effect.

17. The method of claim 15, wherein the second pro-apoptotic drug is a drug that induces apoptosis via an extrinsic pathway.

18. The method of claim 17, wherein the second pro-apoptotic drug induces apoptosis via a Fas pathway.

19. The method of claim 17, wherein the second pro-apoptotic drug induces apoptosis via a c-FLIP pathway.

20. The method of claim 17 wherein the second pro-apoptotic drug is CMH, E2, or δ-tocotrienol.

21. The method of claim 15, wherein the second pro-apoptotic drug is a drug that induces apoptosis via an intrinsic pathway.

22. The method of claim 21, wherein the second pro-apoptotic drug induces apoptosis via a caspase-independent pathway.

23. The method of claim 21, wherein the second pro-apoptotic drug induces apoptosis via a caspase-dependent pathway.

24. The method of claim 21 wherein the second pro-apoptotic drug is E2, FTS, δ-tocotrienol, salinomycin, or curcumin.

25. The method of claim 1, wherein the second pro-apoptotic anticancer drug is FTS, CMH, E2, TMS, δ-tocotrienol, salinomycin, or curcumin.

26. The method of claim 13, wherein the second pro-apoptotic drug induces apoptosis via an extrinsic pathway.

27. The method of claim 26, wherein administering the immunoconjugate and the second pro-apoptotic drug has a synergistic effect.

28. The method of claim 26, wherein the second pro-apoptotic drug is CMH, E2, or δ-tocotrienol.

29. The method of claim 26 wherein the immunoconjugate is T-DM1.

30. The method of claim 13, wherein the second pro-apoptotic induces apoptosis via an intrinsic pathway.

31. The method of claim 30 wherein administering the immunoconjugate and the second pro-apoptotic drug has a synergistic effect.

32. The method of claim 30, wherein the second pro-apoptotic drug is FTS.

33. The method of claim 30, wherein the immunoconjugate is T-DM1.

34. The method of claim 1 comprising treatment of an aromatase-resistant, tamoxifen-resistant, or ER+ hormone refractory breast cancer in a patient afflicted therewith, comprising administering to the patent an effective amount of T-DM1 and an effective amount of FTS, CMH, E2, TMS, δ-tocotrienol, or curcumin, or any combination thereof.

35. The method of claim 1 wherein the method is an adjuvant therapy.

36. The method of claim 1 wherein the method is a first-line therapy.

37. The method of claim 1 wherein the method is a second-line therapy.

38. The method of claim 1 wherein the immunoconjugate and the second pro-apoptotic drug are administered as a combined formulation or by alternation.

39. The method of claim 1 further comprising administering to the patient an additional anticancer drug, wherein optionally the additional anticancer drug exerts an effect via a molecular mechanism different from the molecular mechanism of the first pro-apoptotic anticancer drug moiety and different from the molecular mechanism of the second pro-apoptotic anticancer drug; or administration to the patient of ionizing radiation comprising X-rays, gamma-rays, emissions of radionuclides, or subatomic particles; or any combination thereof.

40. A therapeutic composition comprising (a) an immunoconjugate comprising a monoclonal antibody moiety linked to a first pro-apoptotic drug moiety, and (b) a second pro-apoptotic drug.

41. The composition of claim 40 wherein the covalent immunoconjugate is T-DM1.

42. The composition of claim 40 wherein the second pro-apoptotic anticancer drug is FTS, CMH, E2, TMS, δ-tocotrienol, or curcumin.
